(19) United States
(12) Patent Application Publication
(10) Pub. No.: US 2010/0331312 A1
(43) Pub. Date: Dec. 30, 2010

(54) MODULATORS OF THE HISTAMINE H3 RECEPTOR USEFUL FOR THE TREATMENT OF DISORDERS RELATED THERETO

(75) Inventors: Vincent J. Santora, San Diego, CA (US); Brian J. Hofilena, San Diego, CA (US); Michelle Pulley, New York, NY (US); Graeme Semple, San Diego, CA (US); Yun Shan, San Diego, CA (US); Brian M. Smith, San Diego, CA (US)

Correspondence Address: FISH & RICHARDSON P.C., P.O. BOX 1022, MINNEAPOLIS, MN, 55440-1022, US

(73) Assignee: ARENA PHARMACEUTICALS, INC., San Diego, CA (US)

(21) Appl. No.: 12/867,570

(22) PCT Filed: Feb. 18, 2009
(86) PCT No.: PCT/US09/01022

§ 371 Date: Sep. 13, 2010

Related U.S. Application Data
(60) Provisional application No. 61/066,246, filed on Feb. 19, 2008.
(60) Provisional application No. 61/195,644, filed on Oct. 8, 2008.

(51) Int. Cl. A61K 31/55; C07D 401/10; C07D 403/10; A61P 25/08; A61P 25/24; A61P 3/04; A61P 25/18; A61P 25/28; A61P 11/02; A61K 31/506; C07D 401/14; A61K 31/4725

(52) U.S. Cl. 514/217.01; 546/146; 548/467; 514/256; 544/333; 514/307; 540/594

(57) ABSTRACT

Amide derivatives of Formula (Ia) and pharmaceutical compositions thereof that modulate the activity of the histamine H3 receptor. Compounds of the present invention and pharmaceutical compositions thereof are directed to methods useful in the treatment of histamine H3-associated disorders, such as cognitive disorders, epilepsy, brain trauma, depression, obesity, disorders of sleep and wakefulness such as excessive daytime sleepiness, narcolepsy, shift-work sleep disorder, drowsiness as a side effect from a medication, maintenance of vigilance to aid in the completion of tasks and the like, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, dementia, Alzheimer's disease, pain and the like.

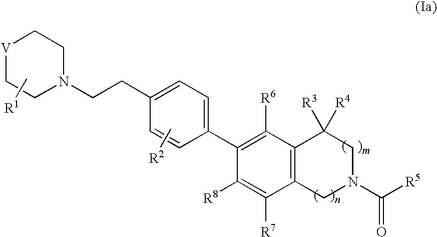

### FIELD OF THE INVENTION

The present invention relates to certain compounds of Formula (Ia) and pharmaceutical compositions thereof that modulate the activity of the histamine H3 receptor. Compounds of the present invention and pharmaceutical compositions thereof are directed to methods useful in the treatment of histamine H3 receptor-associated disorders, such as cognitive disorders, epilepsy, brain trauma, depression, obesity, disorders of sleep and wakefulness such as excessive daytime sleepiness, narcolepsy, shift-work sleep disorder, drowsiness as a side effect from a medication, maintenance of vigilance to aid in the completion of tasks and the like, cataplexy, hypersomnia, somnolence syndrome, jet lag, sleep apnea and the like, attention deficit hyperactivity disorder (ADHD), schizophrenia, allergies, allergic responses in the upper airway, allergic rhinitis, nasal congestion, dementia, Alzheimer's disease, pain and the like.

### SUMMARY OF THE INVENTION

One aspect of the present invention encompasses certain amide derivatives selected from compounds of Formula (Ia) and pharmaceutically acceptable salts, solvates and hydrates thereof:

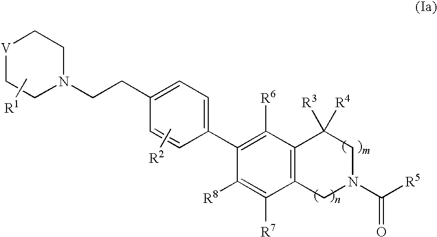

wherein:

R¹ is H or C₁-C₄ alkyl;

R² is H or halogen;

R³ is H, C₁-C₄ alkyl or C₃-C₆ cycloalkyl, and R⁴ is H; or R³ and R⁴ together with the atom to which they are both bonded form a C₃-C₆ cycloalkyl;

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;

R⁶, R⁷ and R⁸ are each independently selected from: H, C₁-C₆ alkoxy, C₁-C₆ alkyl, amino, halogen, heterocyclyl and hydroxyl;

m is 0 or 1;

n is 1 or 2; and

V is CH₂, O or absent.

One aspect of the present invention pertains to methods for inducing wakefulness in an individual comprising administering to the individual in need thereof a therapeutically effective amount of a compound of the present invention or a pharmaceutical composition thereof.

One aspect of the present invention pertains to methods for treating a histamine H3 receptor-associated disorder in an individual comprising administering to the individual in need thereof a therapeutically effective amount of a compound of the present invention or a pharmaceutical composition thereof.

One aspect of the present invention pertains to methods for treating a histamine H3 receptor-associated disorder selected from a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain, comprising administering to the individual in need thereof a therapeutically effective amount of a compound of the present invention or a pharmaceutical composition thereof.

One aspect of the present invention pertains to methods for treating a cognitive disorder in an individual comprising administering to the individual in need thereof a therapeutically effective amount of a compound of the present invention or a pharmaceutical composition thereof.

One aspect of the present invention pertains to methods for treating epilepsy in an individual comprising administering to the individual in need thereof a therapeutically effective amount of a compound of the present invention or a pharmaceutical composition thereof.

One aspect of the present invention pertains to methods for treating a disorder of sleep and wakefulness in an individual comprising administering to the individual in need thereof a therapeutically effective amount of a compound of the present invention or a pharmaceutical composition thereof.

One aspect of the present invention pertains to methods for treating narcolepsy in an individual comprising administering to the individual in need thereof a therapeutically effective amount of a compound of the present invention or a pharmaceutical composition thereof.

One aspect of the present invention pertains to methods for treating shift-work sleep disorder in an individual comprising administering to the individual in need thereof a therapeutically effective amount of a compound of the present invention or a pharmaceutical composition thereof.

One aspect of the present invention pertains to methods for treating cataplexy in an individual comprising administering to the individual in need thereof a therapeutically effective amount of a compound of the present invention or a pharmaceutical composition thereof.

One aspect of the present invention pertains to methods for treating jet lag in an individual comprising administering to the individual in need thereof a therapeutically effective amount of a compound of the present invention or a pharmaceutical composition thereof.

One aspect of the present invention pertains to methods for treating sleep apnea in an individual comprising administering to the individual in need thereof a therapeutically effective amount of a compound of the present invention or a pharmaceutical composition thereof.

One aspect of the present invention pertains to methods for treating excessive daytime sleepiness in an individual comprising administering to the individual in need thereof a therapeutically effective amount of a compound of the present invention or a pharmaceutical composition thereof.

One aspect of the present invention pertains to methods for treating attention deficit hyperactivity disorder in an individual comprising administering to the individual in need thereof a therapeutically effective amount of a compound of the present invention or a pharmaceutical composition thereof.

One aspect of the present invention pertains to methods for treating schizophrenia in an individual comprising administering to the individual in need thereof a therapeutically effective amount of a compound of the present invention or a pharmaceutical composition thereof.

One aspect of the present invention pertains to methods for treating pain in an individual comprising administering to the individual in need thereof a therapeutically effective amount of a compound of the present invention or a pharmaceutical composition thereof.

One aspect of the present invention pertains to the use of a compound of the present invention in the manufacture of a medicament for inducing wakefulness.

One aspect of the present invention pertains to the use of a compound of the present invention in the manufacture of a medicament for the treatment of a histamine H3 receptor-associated disorder.

One aspect of the present invention pertains to the use of a compound of the present invention in the manufacture of a medicament for the treatment of a disorder selected from a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain.

One aspect of the present invention pertains to the use of a compound of the present invention in the manufacture of a medicament for the treatment of a cognitive disorder.

One aspect of the present invention pertains to the use of a compound of the present invention in the manufacture of a medicament for the treatment of epilepsy.

One aspect of the present invention pertains to the use of a compound of the present invention in the manufacture of a medicament for the treatment of a disorder of sleep and wakefulness.

One aspect of the present invention pertains to the use of a compound of the present invention in the manufacture of a medicament for the treatment of narcolepsy.

One aspect of the present invention pertains to the use of a compound of the present invention in the manufacture of a medicament for the treatment of shift-work sleep disorder.

One aspect of the present invention pertains to the use of a compound of the present invention in the manufacture of a medicament for the treatment of cataplexy.

One aspect of the present invention pertains to the use of a compound of the present invention in the manufacture of a medicament for the treatment of jet lag.

One aspect of the present invention pertains to the use of a compound of the present invention in the manufacture of a medicament for the treatment of sleep apnea.

One aspect of the present invention pertains to the use of a compound of the present invention in the manufacture of a medicament for the treatment of excessive daytime sleepiness.

One aspect of the present invention pertains to the use of a compound of the present invention in the manufacture of a medicament for the treatment of attention deficit hyperactivity disorder.

One aspect of the present invention pertains to the use of a compound of the present invention in the manufacture of a medicament for the treatment of schizophrenia.

One aspect of the present invention pertains to the use of a compound of the present invention in the manufacture of a medicament for the treatment of pain.

One aspect of the present invention pertains to compounds of the present invention for use in a method of treatment of the human or animal body by therapy.

One aspect of the present invention pertains to compounds of the present invention for use in a method for inducing wakefulness.

One aspect of the present invention pertains to compounds of the present invention for use in a method for the treatment of a histamine H3 receptor-associated disorder.

One aspect of the present invention pertains to compounds of the present invention for use in a method for the treatment of a histamine H3 receptor-associated disorder selected from a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain.

One aspect of the present invention pertains to compounds of the present invention for use in a method for the treatment of a cognitive disorder.

One aspect of the present invention pertains to compounds of the present invention for use in a method for the treatment of epilepsy.

One aspect of the present invention pertains to compounds of the present invention for use in a method for the treatment of a disorder of sleep and wakefulness.

One aspect of the present invention pertains to compounds of the present invention for use in a method for the treatment of narcolepsy.

One aspect of the present invention pertains to compounds of the present invention for use in a method for the treatment of shift-work sleep disorder.

One aspect of the present invention pertains to compounds of the present invention for use in a method for the treatment of cataplexy

One aspect of the present invention pertains to compounds of the present invention for use in a method for the treatment of jet lag

One aspect of the present invention pertains to compounds of the present invention for use in a method for the treatment of sleep apnea.

One aspect of the present invention pertains to compounds of the present invention for use in a method for the treatment of excessive daytime sleepiness.

One aspect of the present invention pertains to compounds of the present invention for use in a method for the treatment of attention deficit hyperactivity disorder

One aspect of the present invention pertains to compounds of the present invention for use in a method for the treatment of schizophrenia.

One aspect of the present invention pertains to compounds of the present invention for use in a method for the treatment of pain.

One aspect of the present invention pertains to compounds for preparing a composition comprising admixing a compound of the present invention and a pharmaceutically acceptable carrier.

One aspect of the present invention pertains to pharmaceutical compositions comprising a crystalline form of the present invention and a pharmaceutically acceptable carrier.

One aspect of the present invention pertains to methods of inducing wakefulness in an individual comprising administering to said individual in need thereof a therapeutically effective amount of a crystalline form of the present invention or a pharmaceutical composition thereof.

One aspect of the present invention pertains to methods for treating a histamine H3 receptor-associated disorder in an individual comprising administering to said individual in need thereof a therapeutically effective amount of a crystalline form of the present invention or a pharmaceutical composition thereof.

One aspect of the present invention pertains to methods for treating a histamine H3 receptor-associated disorder selected from: a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain in an individual comprising administering to said individual in need thereof a therapeutically effective amount of a crystalline form of the present invention or a pharmaceutical composition thereof.

One aspect of the present invention pertains to methods for treating a cognitive disorder in an individual comprising administering to said individual in need thereof a therapeutically effective amount of a crystalline form of the present invention or a pharmaceutical composition thereof.

One aspect of the present invention pertains to methods for treating epilepsy in an individual comprising administering to said individual in need thereof a therapeutically effective amount of a crystalline form of the present invention or a pharmaceutical composition thereof.

One aspect of the present invention pertains to methods for treating a disorder of sleep and wakefulness in an individual comprising administering to said individual in need thereof a therapeutically effective amount of a crystalline form of the present invention or a pharmaceutical composition thereof.

One aspect of the present invention pertains to methods for treating narcolepsy in an individual comprising administering to said individual in need thereof a therapeutically effective amount of a crystalline form of the present invention or a pharmaceutical composition thereof.

One aspect of the present invention pertains to methods for treating shift-work sleep disorder in an individual comprising administering to said individual in need thereof a therapeutically effective amount of a crystalline form of the present invention or a pharmaceutical composition thereof.

One aspect of the present invention pertains to methods for treating cataplexy in an individual comprising administering to said individual in need thereof a therapeutically effective amount of a crystalline form of the present invention or a pharmaceutical composition thereof.

One aspect of the present invention pertains to methods for treating jet lag in an individual comprising administering to said individual in need thereof a therapeutically effective amount of a crystalline form of the present invention or a pharmaceutical composition thereof.

One aspect of the present invention pertains to methods for treating sleep apnea in an individual comprising administering to said individual in need thereof a therapeutically effective amount of a crystalline form of the present invention or a pharmaceutical composition thereof.

One aspect of the present invention pertains to methods for treating excessive daytime sleepiness in an individual comprising administering to said individual in need thereof a therapeutically effective amount of a crystalline form of the present invention or a pharmaceutical composition thereof.

One aspect of the present invention pertains to methods for treating attention deficit hyperactivity disorder in an individual comprising administering to said individual in need thereof a therapeutically effective amount of a crystalline form of the present invention or a pharmaceutical composition thereof.

One aspect of the present invention pertains to methods for treating schizophrenia in an individual comprising administering to said individual in need thereof a therapeutically effective amount of a crystalline form of the present invention or a pharmaceutical composition thereof.

One aspect of the present invention pertains to methods for treating pain in an individual comprising administering to said individual in need thereof a therapeutically effective amount of a crystalline form of the present invention or a pharmaceutical composition thereof.

Use of a crystalline form of the present invention in the manufacture of a medicament for inducing wakefulness.

Use of a crystalline form of the present invention in the manufacture of a medicament for the treatment of a histamine H3 receptor-associated disorder.

Use of a crystalline form of the present invention in the manufacture of a medicament for the treatment of a disorder selected from: a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain.

Use of a crystalline form of the present invention in the manufacture of a medicament for the treatment of a cognitive disorder.

Use of a crystalline form of the present invention in the manufacture of a medicament for the treatment of epilepsy.

Use of a crystalline form of the present invention in the manufacture of a medicament for the treatment of a disorder of sleep and wakefulness.

Use of a crystalline form of the present invention in the manufacture of a medicament for the treatment of narcolepsy.

Use of a crystalline form of the present invention in the manufacture of a medicament for the treatment of shift-work sleep disorder.

Use of a crystalline form of the present invention in the manufacture of a medicament for the treatment of cataplexy.

Use of a crystalline form of the present invention in the manufacture of a medicament for the treatment of jet lag.

Use of a crystalline form of the present invention in the manufacture of a medicament for the treatment of sleep apnea.

Use of a crystalline form of the present invention in the manufacture of a medicament for the treatment of excessive daytime sleepiness.

Use of a crystalline form of the present invention in the manufacture of a medicament for the treatment of attention deficit hyperactivity disorder.

Use of a crystalline form of the present invention in the manufacture of a medicament for the treatment of schizophrenia.

Use of a crystalline form of the present invention in the manufacture of a medicament for the treatment of pain.

A crystalline form of the present invention for use in a method of treatment of the human or animal body by therapy.

A crystalline form of the present invention for use in a method of inducing wakefulness.

A crystalline form of the present invention for use in a method for the treatment of a histamine H3 receptor-associated disorder.

A crystalline form of the present invention for use in a method for the treatment of a histamine H3 receptor-associated disorder selected from: a cognitive disorder, epilepsy, brain trauma, depression, obesity, disorders of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, allergies, allergic responses in the upper airway, allergic rhinitis, nasal congestion, dementia, Alzheimer's disease and pain.

A crystalline form of the present invention for use in a method for the treatment of a cognitive disorder.

A crystalline form of the present invention for use in a method for the treatment of epilepsy.

A crystalline form of the present invention for use in a method for the treatment of a disorder of sleep and wakefulness.

A crystalline form of the present invention for use in a method for the treatment of narcolepsy.

A crystalline form of the present invention for use in a method for the treatment of shift-work sleep disorder.

A crystalline form of the present invention for use in a method for the treatment of cataplexy.

A crystalline form of the present invention for use in a method for the treatment of jet lag.

A crystalline form of the present invention for use in a method for the treatment of sleep apnea.

A crystalline form of the present invention for use in a method for the treatment of excessive daytime sleepiness.

A crystalline form of the present invention for use in a method for the treatment of attention deficit hyperactivity disorder.

A crystalline form of the present invention for use in a method for the treatment of schizophrenia.

A crystalline form of the present invention for use in a method for the treatment of pain.

A process for preparing a composition comprising admixing a crystalline form of the present invention and a pharmaceutically acceptable carrier.

These and other aspects of the invention disclosed herein will be set forth in greater detail as the patent disclosure proceeds.

### BRIEF DESCRIPTION OF THE DRAWINGS

FIG. 1 shows a general method for preparing compounds of the present invention. First, an aryl boronic acid and a cyclic amine derivative are coupled in the presence of a palladium catalyst. The secondary amino group is acylated with either an acid chloride or a carboxylic acid in the presence of PS-carbodiimide.

FIG. 2 shows a second general method for preparing compounds of the present invention. First, an aryl boronic acid and a Boc-protected cyclic amine derivative are coupled in the presence of a palladium catalyst. The Boc group is removed by acidic hydrolysis and then the secondary amino group is acylated with either an acid chloride or a carboxylic acid in the presence of PS-carbodiimide.

FIG. 3 shows an alternative preparation of compounds of the present invention. First, a cyclic amine is acylated with 2,2-dimethyl-1,3-dioxolan-4-one to form the corresponding 2-hydroxyacetyl derivative. This is converted to a boronate ester which is then coupled with an aryl halide or triflate in the presence of a palladium catalyst.

FIG. 4 shows a method for preparing fluoro substituted aryl triflate intermediates useful in the preparation of compounds of the present invention.

FIG. 5 shows a method for preparing chloro substituted aryl triflate intermediates useful in the preparation of compounds of the present invention.

FIG. 6 shows a general method for preparing compounds of the present invention. A cyclic amine is first acylated by reaction with an acid chloride or a carboxylic acid in the presence of HOBt and EDC. Next the resulting amide is coupled with a boronic acid derivative in the presence of a palladium catalyst to give a compound of Formula (Ia).

FIG. 7 shows a method for preparing compounds of the present invention which are isoindoline derivatives. First an isoindoline-1,3-dione derivative substituted with a halogen is reduced to give the corresponding isoindoline. The isoindoline coupled with a boronic acid in the presence of a palladium catalyst and the secondary amine is then acylated by reaction with either an acid chloride or a carboxylic acid and a coupling agent.

FIG. 8 shows a method for preparing compounds of the present invention which are 1,2,3,4-tetrahydroisoquinoline derivatives. Starting from a 3-methoxybenzaldehyde derivative reaction with nitromethane followed by reduction gives the corresponding 2-aminoethyl intermediate. Alternatively, the 2-aminoethyl intermediate may be prepared directly by reduction of a 2-(3-methoxyphenyl)acetonitrile derivative. Next cyclization is achieved by treatment with formaldehyde and the cyclic amine is acylated with an acid chloride. The methoxy group is converted to a trifluoromethanesulfonyl group by treatment with boron tribromide followed by trifluoromethanesulfonic anhydride and finally coupling with a boronic acid derivative in the presence of a palladium catalyst affords compounds of Formula (Ia) containing a 1,2,3,4-tetrahydroisoquinoline moiety.

FIG. 9 shows another method for preparing compounds of the present invention which are 1,2,3,4-tetrahydroisoquinoline derivatives. Starting from a 2-(3-bromophenyl)ethanamine derivative the amine is converted to the carbamate and cyclized with polyphosphoric acid. Suzuki coupling reduction of the amide and acylation of the secondary amine affords compounds of Formula (Ia) containing a 1,2,3,4-tetrahydroisoquinoline moiety.

FIG. 10 shows two general method for preparing intermediates useful in the synthesis of compounds of the present invention. The first method describes the preparation of substituted 1,2,3,4-tetrahydroisoquinolines useful in the synthesis of compounds of the present invention from 2-(3-methoxyphenyl)ethanamine derivatives by reaction with an acid chloride followed by reduction. The second method describes the preparation of substituted 2-(3-methoxyphenyl)ethanamine derivatives useful in the synthesis of compounds of the present invention from 1-methoxy-3-(2-nitrovinyl)benzene derivatives by treatment with an alkyl lithium followed by reduction.

FIG. 11 shows a synthesis of aryl boronic acid derivatives useful in the preparation of compounds of the present invention. First, a haloaryl ethanol derivative is converted to the mesylate and subsequently coupled with a secondary amine. The halogen is converted to the boronic acid by treatment with triisopropylborate in the presence of a base.

FIG. 12 shows a synthesis of aryl trifluoromethanesulfonate derivatives useful in the preparation of compounds of the present invention. First a methoxyphenylacetic acid derivative is reduced and converted to the mesylate and then a secondary amine is coupled. The methoxy group is converted to the trifluoromethanesulfonate via the alcohol by treatment with boron tribromide followed by trifluoromethanesulfonic anhydride.

FIG. 13 shows an alternative synthesis of certain compounds of the present invention. First, a cyclic amine is reacted with 2-chloro-2-oxoethyl acetate to give an amide which is then coupled to an aryl boronic acid bearing a TBDMS-protected 2-hydroxyethyl group. The reaction proceeds with concurrent ester hydrolysis and desilylation to give a diol, which is treated with base to convert the amide back to an amine and then the alcohol is once again protected using TBDMS chloride. The amine is converted back to the amide by reacting with 2-chloro-2-oxoethyl acetate again. Next, the TBDMS group is hydrolyzed with acid and the resulting alcohol is tosylated. Reaction with a secondary amine followed by acidic hydrolysis of the ester gives compounds of Formula (Ia) containing a 2-hydroxyacetamido group.

FIG. 14 depicts a powder X-ray diffraction pattern (PXRD) for a sample containing a crystalline form of (R)-2-hydroxy-1-(6-(4-(2-(2-methylpyrrolidin-1-yl)ethyl)phenyl)-3,4-dihydroisoquinolin-2(1H)-yl)ethanone hydrochloride (PANalytical X'Pert Plus Powder X-Ray Diffractometer; 5.0 to 40.0°2θ).

FIG. 15 depicts a differential scanning calorimetry (DSC) thermogram for a crystalline form of (R)-2-hydroxy-1-(6-(4-(2-(2-methylpyrrolidin-1-yl)ethyl)phenyl)-3,4-dihydroisoquinolin-2(1H)-yl)ethanone hydrochloride (TA Instruments DSC Q1000; about 25 to about 250° C.; 10° C./min). FIG. 15 also depicts a thermogravimetric analysis (TGA) thermogram for a crystalline form of (R)-2-hydroxy-1-(6-(4-(2-(2-methylpyrrolidin-1-yl)ethyl)phenyl)-3,4-dihydroisoquinolin-2(1H)-yl)ethanone hydrochloride (TA Instruments TGA Q5000 in open cell; 10° C./min).

FIG. 16 depicts a dynamic vapor sorption (DVS) scan for a crystalline form of (R)-2-hydroxy-1-(6-(4-(2-(2-methylpyrrolidin-1-yl)ethyl)phenyl)-3,4-dihydroisoquinolin-2(1H)-yl)ethanone hydrochloride (VTI dynamic vapor sorption analyzer).

### DETAILED DESCRIPTION OF THE INVENTION

### Definitions

For clarity and consistency, the following definitions will be used throughout this patent document.

The term “agonists” is intended to mean moieties that interact and activate the receptor, such as the histamine H3 receptor and initiate a physiological or pharmacological response characteristic of that receptor. For example, when moieties activate the intracellular response upon binding to the receptor, or enhance GTP binding to membranes.

The term “antagonists” is intended to mean moieties that competitively bind to the receptor at the same site as agonists (for example, the endogenous ligand), but which do not activate the intracellular response initiated by the active form of the receptor and can thereby inhibit the intracellular responses by agonists or partial agonists. Antagonists do not diminish the baseline intracellular response in the absence of an agonist or partial agonist.

The term “contact or contacting” is intended to mean bringing the indicated moieties together, whether in an in vitro system or an in vivo system. Thus, “contacting” a histamine H3 receptor with a compound of the invention includes the administration of a compound of the present invention to an individual, preferably a human, having a histamine H3 receptor, as well as, for example, introducing a compound of the invention into a sample containing a cellular or more purified preparation containing a histamine H3 receptor.

The term “hydrate” as used herein means a compound of the invention or a salt thereof, that further includes a stoichiometric or non-stoichiometric amount of water bound by non-covalent intermolecular forces.

The term “in need of treatment” and the term “in need thereof” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit or ameliorate the disease, condition or disorder.

The term “individual” is intended to mean any animal, including mammals, preferably mice, rats, other rodents, rabbits, dogs, cats, swine, cattle, sheep, horses, or primates and most preferably humans.

The term “inverse agonists” is intended to mean moieties that bind to the endogenous form of the receptor or to the constitutively activated form of the receptor and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50% and most preferably by at least 75%, as compared with the baseline response in the absence of the inverse agonist.

The term “modulate or modulating” is intended to mean an increase or decrease in the amount, quality, response or effect of a particular activity, function or molecule.

The term “pharmaceutical composition” is intended to mean a composition comprising at least one active ingredient; including but not limited to, salts, solvates and hydrates of compounds of the present invention; whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The term “solvate” as used herein means a compound of the invention or a salt, thereof, that further includes a stoichiometric or non-stoichiometric amount of a solvent bound by non-covalent intermolecular forces. Preferred solvents are volatile, non-toxic, and/or acceptable for administration to humans in trace amounts.

The term “therapeutically effective amount” is intended to mean the amount of active compound or pharmaceutical agent that elicits the biological or medicinal response in a tissue, system, animal, individual or human that is being sought by a researcher, veterinarian, medical doctor or other clinician or caregiver; or by an individual, which includes one or more of the following:

(1) Preventing the disease; for example, preventing a disease, condition or disorder in an individual that may be predisposed to the disease, condition or disorder but does not yet experience or display the pathology or symptomatology of the disease,

(2) Inhibiting the disease; for example, inhibiting a disease, condition or disorder in an individual that is experiencing or displaying the pathology or symptomatology of the disease, condition or disorder (i.e., arresting further development of the pathology and/or symptomatology) and

(3) Ameliorating the disease; for example, ameliorating a disease, condition or disorder in an individual that is experiencing or displaying the pathology or symptomatology of the disease, condition or disorder (i.e., reversing the pathology and/or symptomatology).

### Chemical Group, Moiety or Radical

The term “C₁-C₄ acyl” is intended to mean a C₁-C₄ alkyl radical attached to the carbon of a carbonyl group wherein the definition of alkyl has the same definition as described herein; some examples include, but are not limited to, acetyl, propionyl, n-butanoyl, t-butanoyl (i.e., pivaloyl), n-pentanoyl, and the like.

The term “C₁-C₆ alkoxy” is intended to mean a C₁-C₆ alkyl radical, as defined herein, attached directly to an oxygen atom, some embodiments are 1 to 5 carbons, some embodiments are 1 to 4 carbons, some embodiments are 1 to 3 carbons and some embodiments are 1 or 2 carbons. Examples include methoxy, ethoxy, n-propoxy, iso-propoxy, n-butoxy, t-butoxy, iso-butoxy, sec-butoxy and the like.

The term “C₁-C₆ alkyl” is intended to mean a straight or branched carbon radical containing 1 to 6 carbons. Some embodiments are 1 to 5 carbons. Some embodiments are 1 to 4 carbons. Some embodiments are 1 to 3 carbons. Some embodiments are 1 or 2 carbons. Some embodiments are 1 carbon. Examples of an alkyl include, but are not limited to, methyl, ethyl, n-propyl, iso-propyl, n-butyl, sec-butyl, iso-butyl, t-butyl, pentyl, iso-pentyl, t-pentyl, neo-pentyl, 1-methylbutyl[i.e., —CH(CH₃)CH₂CH₂CH₃], 2-methylbutyl [i.e., —CH₂CH(CH₃)CH₂CH₃], n-hexyl and the like.

The term “C₁-C₄ alkyl” is intended to mean a straight or branched carbon radical containing 1 to 4 carbons. Some embodiments are 1 to 3 carbons. Some embodiments are 1 or 2 carbons. Some embodiments are 1 carbon. Examples of a C₁-C₄ alkyl include methyl, ethyl, n-propyl, iso-propyl, n-butyl, sec-butyl, iso-butyl and t-butyl.

The term “amino” is intended to mean the group —NH₂.

The term “aryl” is intended to mean an aromatic ring radical containing 6 to 10 ring carbons. Examples include phenyl and naphthyl.

The term “C₃-C₆ cycloalkyl” is intended to mean a saturated ring radical containing 3 to 6 carbons. Some embodiments contain 3 to 5 carbons; some embodiments contain 5 to 6 carbons; some embodiments contain 3 to 4 carbons. Examples include cyclopropyl, cyclobutyl, cyclopentyl, cyclohexyl and the like.

The term “halogen” or “halo” is intended to mean to a fluoro, chloro, bromo or iodo group.

The term “heteroaryl” is intended to mean an aromatic ring system containing 5 to 14 aromatic ring atoms that may be a single ring, two fused rings or three fused rings wherein at least one aromatic ring atom is a heteroatom selected from, for example, but not limited to, the group consisting of O, S and N wherein the N can be optionally substituted with H, C₁-C₄ acyl or C₁-C₄ alkyl. Some embodiments contain 5 to 6 ring atoms for example furanyl, thienyl, pyrrolyl, imidazolyl, oxazolyl, thiazolyl, isoxazolyl, pyrazolyl, isothiazolyl, oxadiazolyl, triazolyl, thiadiazolyl, pyridinyl, pyrazinyl, pyrimidinyl, pyridazinyl and triazinyl and the like. Some embodiments contain 8 to 14 ring atoms for example quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, and isobenzofuran and the like.

The term “heterocyclic” or “heterocyclyl” is intended to mean a non-aromatic, monocyclic ring containing 3 to 8 ring atoms wherein at least one ring atom is a heteroatom or substituted heteroatom selected from, but not limited to, for example, the group consisting of O, S, S(═O), S(═O)₂ and NH, wherein the N is optionally substituted with C₁-C₄ acyl or C₁-C₄ alkyl. In some embodiments, the ring carbon atoms are optionally substituted with oxo thus forming a carbonyl group. In some embodiments the heterocyclic group is a 3-, 4-, 5-, 6- or 7-membered ring. Examples of a heterocyclic group include, but are not limited to, aziridin-2-yl, azetidin-2-yl, azetidin-3-yl, piperidin-2-yl, piperidin-3-yl, piperidin-4-yl, morpholin-2-yl, morpholin-3-yl, piperzin-2-yl, piperzin-3-yl, pyrrolidin-2-yl, pyrrolidin-3-yl, [1,3]-dioxolan-2-yl, azepan-2-yl, azepan-3-yl, azepan-4-yl, tetrahydrofuran-2-yl, tetrahydrofuran-3-yl, tetrahydropyran-2-yl, tetrahydropyran-3-yl and tetrahydropyran-4-yl and the like. It is understood that a heterocyclic group can be bonded only at any available ring carbon as allowed by the respective formulae unless otherwise specified.

The term “hydroxyl” is intended to mean the group —OH.

### Compounds of the Invention:

One aspect of the present invention pertains to certain compounds as shown in Formula (Ia):

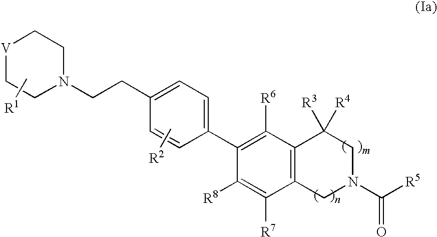

and pharmaceutically acceptable salts, solvates and hydrates thereof;

wherein:

R¹, R², R³, R⁴, R⁵, R⁶, R⁷, R⁸, V, m and n have the same definitions as described herein, supra and infra.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables (e.g., R¹, R², R³, R⁴, R⁵, R⁶, R⁷, R⁸, R⁹, R¹⁰, V, m and n) contained within the generic chemical formulae described herein, for example, Ia, Ic, Ie, Ig, Ii, Ik, Im, Io, Iq, Is, Iu and Iw, are specifically embraced by the present invention just as if each and every combination was individually explicitly recited, to the extent that such combinations embrace compounds that result in stable compounds (i.e., compounds that can be isolated, characterized and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents and the like. Likewise, “substituted with one or more substituents” refers to the substitution of a group with one substituent up to the total number of substituents physically allowed by the group. Further, when a group is substituted with more than one group they can be identical or they can be different.

Compounds of the invention can also include tautomeric forms, such as keto-enol tautomers and the like. Tautomeric forms can be in equilibrium or sterically locked into one form by appropriate substitution. It is understood that the various tautomeric forms are within the scope of the compounds of the present invention.

Compounds of the invention can also include all isotopes of atoms occurring in the intermediates and/or final compounds. Isotopes include those atoms having the same atomic number but different mass numbers. For example, isotopes of hydrogen include deuterium and tritium.

It is understood and appreciated that compounds of Formula (Ia) and formulae related thereto may have one or more chiral centers and therefore can exist as enantiomers and/or diastereoisomers. The invention is understood to extend to and embrace all such enantiomers, diastereoisomers and mixtures thereof, including but not limited to racemates. It is understood that compounds of Formula (Ia) and formulae used throughout this disclosure are intended to represent all individual enantiomers and mixtures thereof, unless stated or shown otherwise.

The Group R¹:

In some embodiments, R¹ is H or C₁-C₄ alkyl.

In some embodiments, R¹ is H.

In some embodiments, R¹ is C₁-C₄ alkyl.

In some embodiments, R¹ is methyl.

In some embodiments, R¹ is ethyl.

In some embodiments, R¹ is isopropyl.

The Group R²:

In some embodiments, R² is H or halogen.

In some embodiments, R² is H.

In some embodiments, R² is halogen.

In some embodiments, R² is fluoro or chloro.

In some embodiments, R² is fluoro.

In some embodiments, R² is chloro.

In some embodiments, R² is bromo.

In some embodiments, R² is iodo.

The Groups R³ and R⁴:

In some embodiments, R³ is H, C₁-C₄ alkyl or C₃-C₆ cycloalkyl, and R⁴ is H.

In some embodiments, R³ is C₁-C₄ alkyl and R⁴ is H.

In some embodiments, R³ is methyl and R⁴ is H.

In some embodiments, R³ is ethyl and R⁴ is H.

In some embodiments, R³ is isopropyl and R⁴ is H.

In some embodiments, R³ is C₃-C₆ cycloalkyl and R⁴ is H.

In some embodiments, R³ is cyclopropyl and R⁴ is H.

In some embodiments, R³ is cyclobutyl and R⁴ is H.

In some embodiments, R³ is cyclopentyl and R⁴ is H.

In some embodiments, R³ is cyclohexyl and R⁴ is H.

In some embodiments, R³ and R⁴ together with the atom to which they are both bonded form a C₃-C₆ cycloalkyl.

In some embodiments, R³ and R⁴ together with the atom to which they are both bonded form cyclopropyl.

In some embodiments, R³ and R⁴ together with the atom to which they are both bonded form cyclobutyl.

In some embodiments, R³ and R⁴ together with the atom to which they are both bonded form cyclopentyl.

In some embodiments, R³ and R⁴ together with the atom to which they are both bonded form cyclohexyl.

In some embodiments, R³ and R⁴ are both H.

The Group R⁵:

In some embodiments, R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

In some embodiments, R⁵ is selected from: methyl, ethyl, n-propyl, cyclopropyl, phenyl, pyridyl, pyrimidinyl and tetrahydropyranyl; each of which is optionally substituted with one or more substituents selected from: C₁-C₆ alkoxy, halogen, heterocyclyl and hydroxyl.

In some embodiments, R⁵ is selected from: methyl, ethyl, n-propyl, cyclopropyl, phenyl, pyridyl, pyrimidinyl and tetrahydropyranyl; each of which is optionally substituted with one or more substituents selected from: methoxy, fluoro, tetrahydropyranyl and hydroxyl.

In some embodiments, R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

In some embodiments, R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

In some embodiments, R⁵ is C₁-C₆ alkyl.

In some embodiments, R⁵ is methyl.

In some embodiments, R⁵ is C₃-C₆ cycloalkyl.

In some embodiments, R⁵ is cyclopropyl.

In some embodiments, R⁵ is heterocyclyl.

In some embodiments, R⁵ is tetrahydropyran-4-yl.

In some embodiments, R⁵ is C₁-C₆ alkyl optionally substituted with C₁-C₆ alkoxy.

In some embodiments, R⁵ is methoxymethyl.

In some embodiments, R⁵ is 2-methoxyethyl.

In some embodiments, R⁵ is 3-methoxypropyl.

In some embodiments, R⁵ is C₁-C₆ alkyl optionally substituted with hydroxyl.

In some embodiments, R⁵ is hydroxymethyl.

In some embodiments, R⁵ is 2-hydroxyethyl.

In some embodiments, R⁵ is C₁-C₆ alkyl optionally substituted with heterocyclyl.

In some embodiments, R⁵ is tetrahydropyran-4-ylmethyl.

In some embodiments, R⁵ is C₃-C₆ cycloalkyl optionally substituted with halogen.

In some embodiments, R⁵ is 2,2-difluorocyclopropyl.

In some embodiments, R⁵ is aryl optionally substituted with C₁-C₆ alkoxy.

In some embodiments, R⁵ is 4-methoxyphenyl.

In some embodiments, R⁵ is heteroaryl.

In some embodiments, R⁵ is pyridin-2-yl.

In some embodiments, R⁵ is pyridin-3-yl.

In some embodiments, R⁵ is pyridin-4-yl.

In some embodiments, R⁵ is pyrimidin-5-yl.

In some embodiments, R⁵ is heteroaryl optionally substituted with hydroxyl.

In some embodiments, R⁵ is 6-hydroxypyridin-3-yl.

In some embodiments, R⁵ is 2-hydroxypyridin-4-yl.

In some embodiments, R⁵ is 6-hydroxypyridin-2-yl.

In some embodiments, R⁵ is 6-methoxypyridin-3-yl.

The Group R⁶:

In some embodiments, R⁶ is selected from: H, C₁-C₆ alkoxy, C₁-C₆ alkyl, amino, halogen, heterocyclyl and hydroxyl.

In some embodiments, R⁶ is selected from: H, methoxy, methyl, fluoro, chloro and bromo.

In some embodiments, R⁶ is selected from: H, methoxy, methyl, fluoro, chloro, bromo and hydroxyl.

In some embodiments, R⁶ is H.

In some embodiments, R⁶ is C₁-C₆ alkoxy.

In some embodiments, R⁶ is methoxy.

In some embodiments, R⁶ is C₁-C₆ alkyl.

In some embodiments, R⁶ is methyl.

In some embodiments, R⁶ is amino.

In some embodiments, R⁶ is halogen.

In some embodiments, R⁶ is fluoro.

In some embodiments, R⁶ is chloro.

In some embodiments, R⁶ is bromo.

In some embodiments, R⁶ is heterocyclyl.

In some embodiments, R⁶ is hydroxyl.

The Group R⁷:

In some embodiments, R⁷ is selected from: H, C₁-C₆ alkoxy, C₁-C₆ alkyl, amino, halogen, heterocyclyl and hydroxyl.

In some embodiments, R⁷ is selected from: H, methoxy, methyl, fluoro, chloro and bromo.

In some embodiments, R⁷ is selected from: H, methoxy, methyl, fluoro, chloro, bromo and hydroxyl.

In some embodiments, R⁷ is H.

In some embodiments, R⁷ is C₁-C₇ alkoxy.

In some embodiments, R⁷ is methoxy.

In some embodiments, R⁷ is C₁-C₇ alkyl.

In some embodiments, R⁷ is methyl.

In some embodiments, R⁷ is amino.

In some embodiments, R⁷ is halogen.

In some embodiments, R⁷ is fluoro.

In some embodiments, R⁷ is chloro.

In some embodiments, R⁷ is bromo.

In some embodiments, R⁷ is heterocyclyl.

In some embodiments, R⁷ is hydroxyl.

The Group R⁸:

In some embodiments, R⁸ is selected from: H, C₁-C₆ alkoxy, C₁-C₆ alkyl, amino, halogen, heterocyclyl and hydroxyl.

In some embodiments, R⁸ is selected from: H, methoxy, methyl, fluoro, chloro and bromo.

In some embodiments, R⁸ is selected from: H, methoxy, methyl, fluoro, chloro, bromo and hydroxyl.

In some embodiments, R⁸ is H.

In some embodiments, R⁸ is C₁-C₈ alkoxy.

In some embodiments, R⁸ is methoxy.

In some embodiments, R⁸ is C₁-C₈ alkyl.

In some embodiments, R⁸ is methyl.

In some embodiments, R⁸ is amino.

In some embodiments, R⁸ is halogen.

In some embodiments, R⁸ is fluoro.

In some embodiments, R⁸ is chloro.

In some embodiments, R⁸ is bromo.

In some embodiments, R⁸ is heterocyclyl.

In some embodiments, R⁸ is hydroxyl.

The Group V:

In some embodiments, V is CH₂, O or absent.

In some embodiments, V is CH₂.

In some embodiments, V is O.

In some embodiments, V is absent.

The Variable m:

In some embodiments, m is 0 or 1.

In some embodiments, m is 0.

In some embodiments, m is 1.

The Variable n:

In some embodiments, n is 1 or 2.

In some embodiments, n is 1.

In some embodiments, n is 2.

Certain Combinations of the Present Invention:

In some embodiments, R⁶, R⁷ and R⁸ are each independently selected from: H, C₁-C₆ alkoxy, C₁-C₆ alkyl, amino, halogen, heterocyclyl and hydroxyl.

In some embodiments, R⁶, R⁷ and R⁸ are each independently selected from: H, methoxy, methyl, fluoro, chloro and bromo.

In some embodiments, R⁶, R⁷ and R⁸ are each independently selected from: H, methoxy, methyl, fluoro, chloro, bromo and hydroxyl.

In some embodiments, R⁶, R⁷ and R⁸ are all H.

Some embodiments of the present invention pertain to compounds of Formula (Ic) and pharmaceutically acceptable salts, solvates and hydrates thereof:

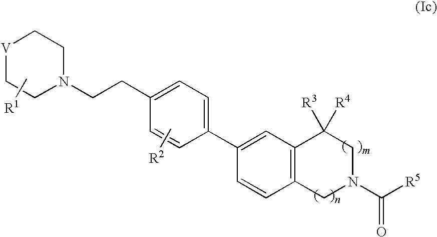

wherein:

R¹ is H or C₁-C₄ alkyl;

R² is H or halogen;

R³ is H, C₁-C₄ alkyl or C₃-C₆ cycloalkyl, and R⁴ is H; or R³ and R⁴ together with the atom to which they are both bonded form a C₃-C₆ cycloalkyl;

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;

m is 0 or 1;

n is 1 or 2; and

V is CH₂, O or absent.

Some embodiments of the present invention pertain to compounds of Formula (Ic) and pharmaceutically acceptable salts, solvates and hydrates thereof:

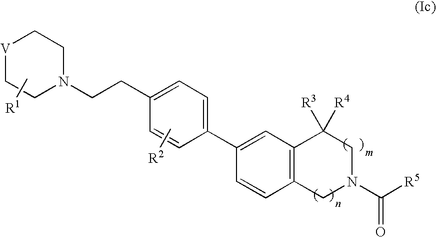

wherein:

R¹ is H or methyl;

R² is H, fluoro or chloro;

R³ is H or methyl;

R⁴ is H;

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl;

m is 0 or 1;

n is 1 or 2; and

V is CH₂ or absent.

Some embodiments of the present invention pertain to compounds of Formula (Ie) and pharmaceutically acceptable salts, solvates and hydrates thereof:

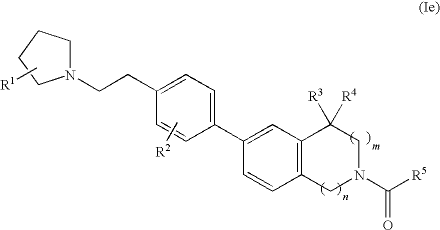

wherein:

R¹ is H or C₁-C₄ alkyl;

R² is H or halogen;

R³ is H, C₁-C₄ alkyl or C₃-C₆ cycloalkyl, and R⁴ is H; or R³ and R⁴ together with the atom to which they are both bonded form a C₃-C₆ cycloalkyl;

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;

m is 0 or 1; and

n is 1 or 2.

Some embodiments of the present invention pertain to compounds of Formula (Ie) and pharmaceutically acceptable salts, solvates and hydrates thereof:

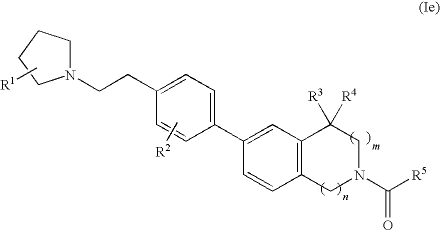

wherein:

R¹ is H or methyl;

R² is H, fluoro or chloro;

R³ is H or methyl;

R⁴ is H;

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-3-yl, pyridin-4-yl, pyrimidin-5-yl, 6-hydroxypyridin-3-yl and 2-hydroxypyridin-4-yl;

m is 0 or 1; and

n is 1 or 2.

Some embodiments of the present invention pertain to compounds of Formula (Ig) and pharmaceutically acceptable salts, solvates and hydrates thereof:

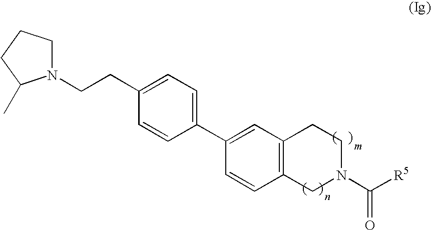

wherein:

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;

m is 0 or 1; and

n is 1 or 2.

Some embodiments of the present invention pertain to compounds of Formula (Ig) and pharmaceutically acceptable salts, solvates and hydrates thereof:

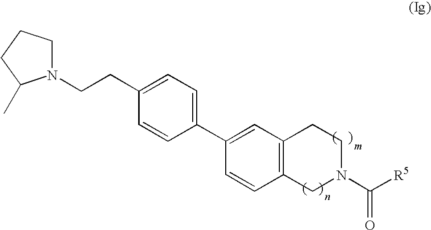

wherein:

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl;

m is 0 or 1; and

n is 1 or 2.

Some embodiments of the present invention pertain to compounds of Formula (Ii) and pharmaceutically acceptable salts, solvates and hydrates thereof:

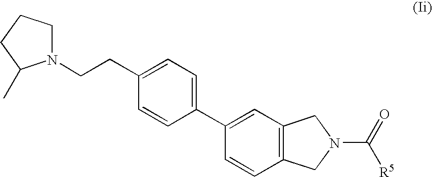

wherein:

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Ii) and pharmaceutically acceptable salts, solvates and hydrates thereof:

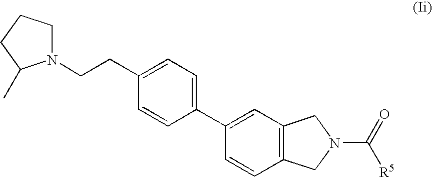

wherein:

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

Some embodiments of the present invention pertain to compounds of Formula (Ik) and pharmaceutically acceptable salts, solvates and hydrates thereof:

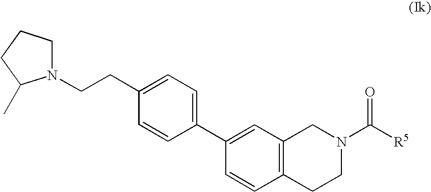

wherein:

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Ik) and pharmaceutically acceptable salts, solvates and hydrates thereof:

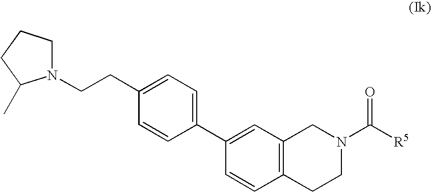

wherein:

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

Some embodiments of the present invention pertain to compounds of Formula (Im) and pharmaceutically acceptable salts, solvates and hydrates thereof:

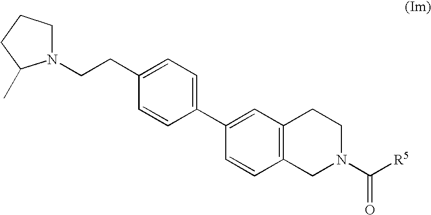

wherein:

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Im) and pharmaceutically acceptable salts, solvates and hydrates thereof:

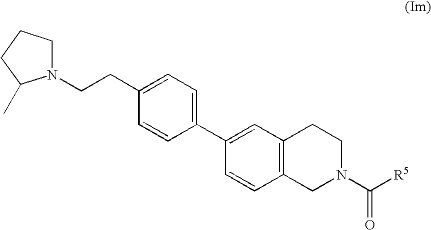

wherein:

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

Some embodiments of the present invention pertain to compounds of Formula (Io) and pharmaceutically acceptable salts, solvates and hydrates thereof:

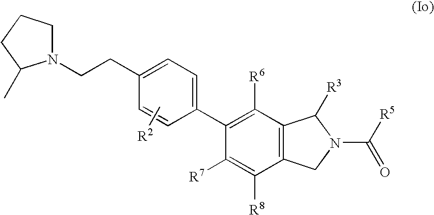

wherein:
R² is H or halogen;
R³ is H or C₁-C₄ alkyl;
R⁶, R⁷ and R⁸ are each independently selected from: H, C₁-C₆ alkoxy, C₁-C₆ alkyl, halogen and hydroxyl; and
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Io) and pharmaceutically acceptable salts, solvates and hydrates thereof:

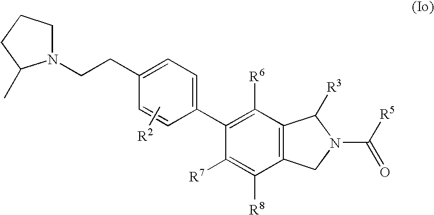

wherein:
R² is H, fluoro or chloro;
R³ is H or methyl;
R⁶, R⁷ and R⁸ are each independently selected from: H, methoxy, methyl, fluoro, chloro and hydroxyl; and
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

Some embodiments of the present invention pertain to compounds of Formula (Iq) and pharmaceutically acceptable salts, solvates and hydrates thereof:

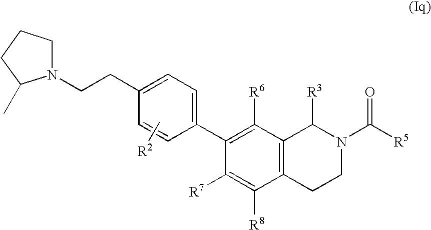

wherein:
R² is H or halogen;

R³ is H or C₁-C₄ alkyl;
R⁶, R⁷ and R⁸ are each independently selected from: H, C₁-C₆ alkoxy, C₁-C₆ alkyl, halogen and hydroxyl; and
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Iq) and pharmaceutically acceptable salts, solvates and hydrates thereof:

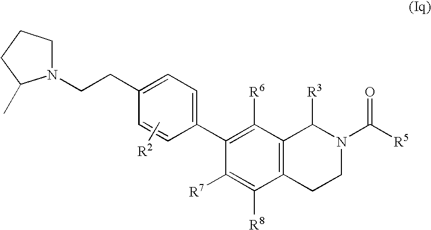

wherein:
R² is H, fluoro or chloro;
R³ is H or methyl;
R⁶, R⁷ and R⁸ are each independently selected from: H, methoxy, methyl, fluoro, chloro and hydroxyl; and
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

Some embodiments of the present invention pertain to compounds of Formula (Is) and pharmaceutically acceptable salts, solvates and hydrates thereof:

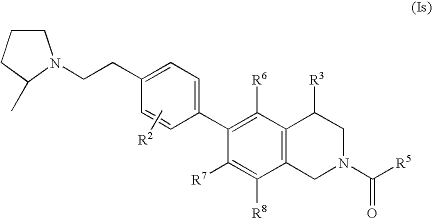

wherein:
R² is H or halogen;
R³ is H or C₁-C₄ alkyl;
R⁶, R⁷ and R⁸ are each independently selected from: H, C₁-C₆ alkoxy, C₁-C₆ alkyl, halogen and hydroxyl; and
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Is) and pharmaceutically acceptable salts, solvates and hydrates thereof:

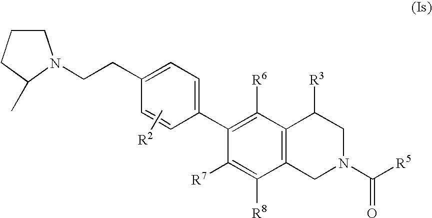

wherein:
R² is H, fluoro or chloro;
R³ is H or methyl;
R⁶, R⁷ and R⁸ are each independently selected from: H, methoxy, methyl, fluoro, chloro and hydroxyl; and
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

Some embodiments of the present invention pertain to compounds of Formula (Iu) and pharmaceutically acceptable salts, solvates and hydrates thereof:

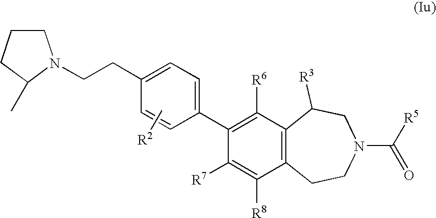

wherein:
R² is H or halogen;
R³ is H or C₁-C₄ alkyl;
R⁶, R⁷ and R⁸ are each independently selected from: H, C₁-C₆ alkoxy, C₁-C₆ alkyl, halogen and hydroxyl; and
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

Some embodiments of the present invention pertain to compounds of Formula (Iu) and pharmaceutically acceptable salts, solvates and hydrates thereof:

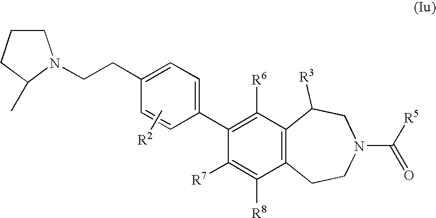

wherein:
R² is H, fluoro or chloro;
R³ is H or methyl;
R⁶, R⁷ and R⁸ are each independently selected from: H, methoxy, methyl, fluoro, chloro and hydroxyl; and
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

Some embodiments of the present invention pertain to compounds of Formula (Iw) and pharmaceutically acceptable salts, solvates and hydrates thereof:

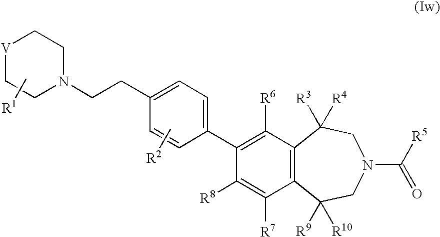

wherein:

R¹ is H or C₁-C₄ alkyl;

R² is H or halogen;

R³ is H, C₁-C₄ alkyl or C₃-C₆ cycloalkyl, and R⁴ is H; or R³ and R⁴ together with the atom to which they are both bonded form a C₃-C₆ cycloalkyl;

R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;

R⁶, R⁷ and R⁸ are each independently selected from: H, C₁-C₆ alkoxy, C₁-C₆ alkyl, amino, halogen, heterocyclyl and hydroxyl;

R⁹ is H, C₁-C₄ alkyl or C₃-C₆ cycloalkyl, and R¹⁰ is H; or R⁹ and R¹⁰ together with the atom to which they are both bonded form a C₃-C₆ cycloalkyl

p is 0 or 1;

q is 0 or 1; and

V is CH₂, O or absent.

Some embodiments of the present invention pertain to compounds of Formula (Iw) and pharmaceutically acceptable salts, solvates and hydrates thereof:

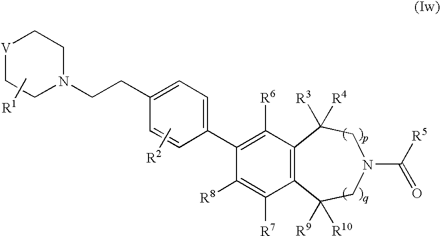

wherein:

R¹ is H or methyl;

R² is H, fluoro or chloro;

R³ is H or methyl;

R⁴ is H;

R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

R⁶, R⁷ and R⁸ are each independently selected from: H, methoxy, methyl, fluoro, chloro and hydroxyl;

R⁹ is H or methyl;

R¹⁰ is H;

p is 0 or 1;

q is 0 or 1; and

V is CH₂ or absent.

Some embodiments of the present invention include every combination of one or more compounds selected from the following group:
3-methoxy-1-(7-(4-(2-(2-methylpyrrolidin-1-yl)ethyl)phenyl)-3,4-dihydroisoquinolin-2(1H)-yl)propan-1-one;
cyclopropyl(7-(4-(2-(2-methylpyrrolidin-1-yl)ethyl)phenyl)-3,4-dihydroisoquinolin-2(1H)-yl)methanone;
cyclopropyl(6-(4-(2-(2-methylpyrrolidin-1-yl)ethyl)phenyl)-3,4-dihydroisoquinolin-2(1H)-yl)methanone;
3-methoxy-1-(6-(4-(2-(2-methylpyrrolidin-1-yl)ethyl)phenyl)-3,4-dihydroisoquinolin-2(1H)-yl)propan-1-one;
cyclopropyl(5-(4-(2-(2-methylpyrrolidin-1-yl)ethyl)phenyl)isoindolin-2-yl)methanone;
3-methoxy-1-(5-(4-(2-(2-methylpyrrolidin-1-yl)ethyl)phenyl)isoindolin-2-yl)propan-1-one;
(6-(4-(2-(2-methylpyrrolidin-1-yl)ethyl)phenyl)-3,4-dihydroisoquinolin-2(1H)-yl)(tetrahydro-2H-pyran-4-yl)methanone;
(2,2-difluorocyclopropyl)(6-(4-(2-(2-methylpyrrolidin-1-yl)ethyl)phenyl)-3,4-dihydroisoquinolin-2(1H)-yl)methanone;
(4-methoxyphenyl)(6-(4-(2-(2-methylpyrrolidin-1-yl)ethyl)phenyl)-3,4-dihydroisoquinolin-2(1H)-yl)methanone;
2-hydroxy-1-(6-(4-(2-(2-methylpyrrolidin-1-yl)ethyl)phenyl)-3,4-dihydroisoquinolin-2(1H)-yl)ethanone;
1-(6-(4-(2-(2-methylpyrrolidin-1-yl)ethyl)phenyl)-3,4-dihydroisoquinolin-2(1H)-yl)-2-(tetrahydro-2H-pyran-4-yl)ethanone;
(5-(4-(2-(2-methylpyrrolidin-1-yl)ethyl)phenyl)isoindolin-2-yl)(tetrahydro-2H-pyran-4-yl)methanone;
(6-(4-(2-(2-methylpyrrolidin-1-yl)ethyl)phenyl)-3,4-dihydroisoquinolin-2(1H)-yl)(pyridin-3-yl)methanone;
(6-(4-(2-(2-methylpyrrolidin-1-yl)ethyl)phenyl)-3,4-dihydroisoquinolin-2(1H)-yl)(pyridin-4-yl)methanone;
(6-(4-(2-(2-methylpyrrolidin-1-yl)ethyl)phenyl)-3,4-dihydroisoquinolin-2(1H)-yl)(pyrimidin-5-yl)methanone;
3-hydroxy-1-(6-(4-(2-(2-methylpyrrolidin-1-yl)ethyl)phenyl)-3,4-dihydroisoquinolin-2(1H)-yl)propan-1-one;
4-methoxy-1-(5-(4-(2-(2-methylpyrrolidin-1-yl)ethyl)phenyl)isoindolin-2-yl)butan-1-one;
(6-(4-(2-(2-methylpyrrolidin-1-yl)ethyl)phenyl)-3,4-dihydroisoquinolin-2(1H)-yl)(pyridin-2-yl)methanone;
2-methoxy-1-(6-(4-(2-(2-methylpyrrolidin-1-yl)ethyl)phenyl)-3,4-dihydroisoquinolin-2(1H)-yl)ethanone;
4-methoxy-1-(6-(4-(2-(2-methylpyrrolidin-1-yl)ethyl)phenyl)-3,4-dihydroisoquinolin-2(1H)-yl)butan-1-one;
(6-hydroxypyridin-3-yl)(6-(4-(2-(2-methylpyrrolidin-1-yl)ethyl)phenyl)-3,4-dihydroisoquinolin-2(1H)-yl)methanone;
(2-hydroxypyridin-4-yl)(6-(4-(2-(2-methylpyrrolidin-1-yl)ethyl)phenyl)-3,4-dihydroisoquinolin-2(1H)-yl)methanone; and
1-(6-(4-(2-(2-methylpyrrolidin-1-yl)ethyl)phenyl)-3,4-dihydroisoquinolin-2(1H)-yl)ethanone.

Some embodiments of the present invention include every combination of one or more compounds selected from the combined group of compounds comprising the group of compounds immediately preceding this sentence and the following group:
1-(1-methyl-8-(4-(2-(2-methylpyrrolidin-1-yl)ethyl)phenyl)-4,5-dihydro-1H-benzo[d]azepin-3(2H)-yl)ethanone;
(5-(4-(2-(2-methylpyrrolidin-1-yl)ethyl)phenyl)isoindolin-2-yl)(pyridin-4-yl)methanone;
3-methoxy-1-(1-methyl-8-(4-(2-(2-methylpyrrolidin-1-yl)ethyl)phenyl)-4,5-dihydro-1H-benzo[d]azepin-3(2H)-yl)propan-1-one;
cyclopropyl(1-methyl-8-(4-(2-(2-methylpyrrolidin-1-yl)ethyl)phenyl)-4,5-dihydro-1H-benzo[d]azepin-3(2H)-yl)methanone;
(5-(4-(2-(2-methylpyrrolidin-1-yl)ethyl)phenyl)isoindolin-2-yl)(pyridin-3-yl)methanone;
(5-(4-(2-(2-methylpyrrolidin-1-yl)ethyl)phenyl)isoindolin-2-yl)(pyrimidin-5-yl)methanone;
(5-(4-(2-(2-methylpyrrolidin-1-yl)ethyl)phenyl)isoindolin-2-yl)(pyridin-2-yl)methanone;
(6-hydroxypyridin-2-yl)(6-(4-(2-(2-methylpyrrolidin-1-yl)ethyl)phenyl)-3,4-dihydroisoquinolin-2(1H)-yl)methanone;
(6-methoxypyridin-3-yl)(6-(4-(2-(2-methylpyrrolidin-1-yl)ethyl)phenyl)-3,4-dihydroisoquinolin-2(1H)-yl)methanone;
2-methoxy-1-(5-(4-(2-(2-methylpyrrolidin-1-yl)ethyl)phenyl)isoindolin-2-yl)ethanone;
2-hydroxy-1-(5-(4-(2-(2-methylpyrrolidin-1-yl)ethyl)phenyl)isoindolin-2-yl)ethanone;
1-(9-fluoro-1-methyl-8-(4-(2-(2-methylpyrrolidin-1-yl)ethyl)phenyl)-4,5-dihydro-1H-benzo[d]azepin-3(2H)-yl)ethanone;
1-(9-chloro-1-methyl-7-(4-(2-(2-methylpyrrolidin-1-yl)ethyl)phenyl)-4,5-dihydro-1H-benzo[d]azepin-3(2H)-yl)ethanone;
1-(5-chloro-6-(4-(2-(2-methylpyrrolidin-1-yl)ethyl)phenyl)-3,4-dihydroisoquinolin-2(1H)-yl)ethanone;
1-(5-fluoro-6-(4-(2-(2-methylpyrrolidin-1-yl)ethyl)phenyl)-3,4-dihydroisoquinolin-2(1H)-yl)ethanone;
1-(7-hydroxy-1-methyl-8-(4-(2-(2-methylpyrrolidin-1-yl)ethyl)phenyl)-4,5-dihydro-1H-benzo[d]azepin-3 (2H)-yl)ethanone;
1-(7-methyl-6-(4-(2-(2-methylpyrrolidin-1-yl)ethyl)phenyl)-3,4-dihydroisoquinolin-2(1H)-yl)ethanone;
1-(7-methoxy-1-methyl-8-(4-(2-(2-methylpyrrolidin-1-yl)ethyl)phenyl)-4,5-dihydro-1H-benzo[d]azepin-3(2H)-yl)ethanone;
1-(7-(4-(2-(2-methylpyrrolidin-1-yl)ethyl)phenyl)-4,5-dihydro-1H-benzo[d]azepin-3(2H)-yl)ethanone;
2-hydroxy-1-(6-(4-(2-(piperidin-1-yl)ethyl)phenyl)-3,4-dihydroisoquinolin-2(1H)-yl)ethanone;
1-(7-methoxy-6-(4-(2-(2-methylpyrrolidin-1-yl)ethyl)phenyl)-3,4-dihydroisoquinolin-2(1H)-yl)ethanone;
2-hydroxy-1-(6-(4-(2-morpholinoethyl)phenyl)-3,4-dihydroisoquinolin-2(1H)-yl)ethanone;
1-(7-hydroxy-6-(4-(2-(2-methylpyrrolidin-1-yl)ethyl)phenyl)-3,4-dihydroisoquinolin-2(1H)-yl)ethanone;
1-(1-methyl-6-(4-(2-(2-methylpyrrolidin-1-yl)ethyl)phenyl)-3,4-dihydroisoquinolin-2(1H)-yl)ethanone;
2-hydroxy-1-(7-(4-(2-(2-methylpyrrolidin-1-yl)ethyl)phenyl)-4,5-dihydro-1H-benzo[d]azepin-3(2H)-yl)ethanone;
1-(7-fluoro-6-(4-(2-(2-methylpyrrolidin-1-yl)ethyl)phenyl)-3,4-dihydroisoquinolin-2(1H)-yl)ethanone;
2-hydroxy-1-(6-(4-(2-(2-methylpyrrolidin-1-yl)ethyl)phenyl)-3,4-dihydroisoquinolin-2(1H)-yl)ethanone;
1-(4-methyl-6-(4-(2-(2-methylpyrrolidin-1-yl)ethyl)phenyl)-3,4-dihydroisoquinolin-2(1H)-yl)ethanone;
1-(6-(3-fluoro-4-(2-(2-methylpyrrolidin-1-yl)ethyl)phenyl)-3,4-dihydroisoquinolin-2(1H)-yl)ethanone; and
1-(6-(2-chloro-4-(2-(2-methylpyrrolidin-1-yl)ethyl)phenyl)-3,4-dihydroisoquinolin-2(1H)-yl)ethanone.

Some embodiments of the present invention include every combination of one or more compounds selected from the following group shown in TABLE A and TABLE B.

TABLE A

| Cmpd No. | Chemical Structure | Chemical Name |
|---|---|---|
| 1 | 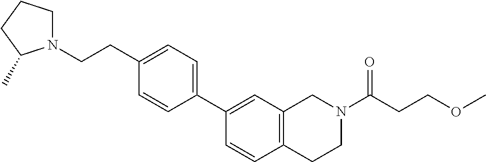 | (R)-3-methoxy-1-(7-(4-(2- (2-methylpyrrolidin-1- yl)ethyl)phenyl)-3,4- dihydroisoquinolin-2(1H)- yl)propan-1-one |
| 2 | 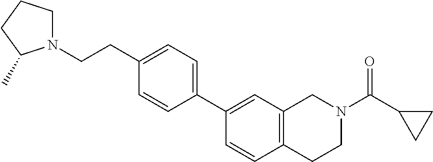 | (R)-cyclopropyl(7-(4-(2-(2- methylpyrrolidin-1- yl)ethyl)phenyl)-3,4- dihydroisoquinolin-2(1H)- yl)methanone |
| 3 | 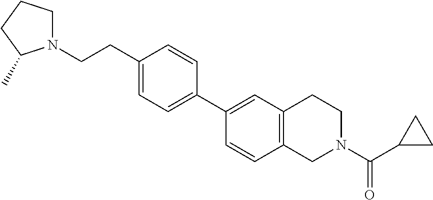 | (R)-cyclopropyl(6-(4-(2-(2- methylpyrrolidin-1- yl)ethyl)phenyl)-3,4- dihydroisoquinolin-2(1H)- yl)methanone |
| 4 | 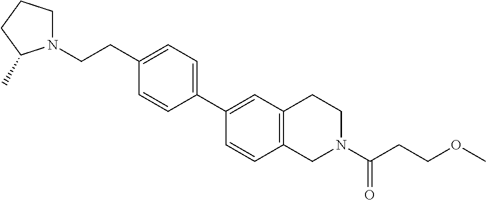 | (R)-3-methoxy-1-(6-(4-(2- (2-methylpyrrolidin-1- yl)ethyl)phenyl)-3,4- dihydroisoquinolin-2(1H)- yl)propan-1-one |
| 5 | 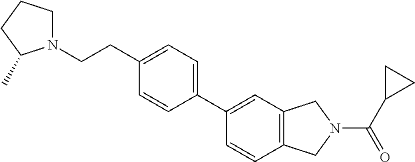 | (R)-cyclopropyl(5-(4-(2-(2- methylpyrrolidin-1- yl)ethyl)phenyl)isoindolin-2- yl)methanone |
| 6 | 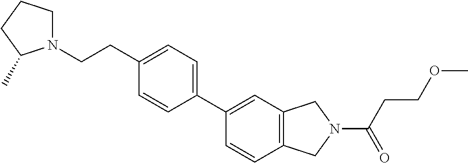 | (R)-3-methoxy-1-(5-(4-(2- (2-methylpyrrolidin-1- yl)ethyl)phenyl)isoindolin-2- yl)propan-1-one |
| 7 | 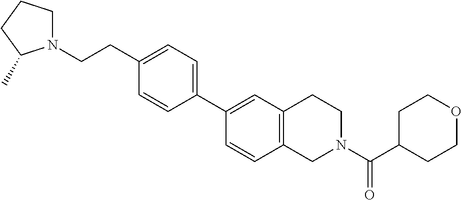 | (R)-(6-(4-(2-(2- methylpyrrolidin-1- yl)ethyl)phenyl)-3,4- dihydroisoquinolin-2(1H)- yl)(tetrahydro-2H-pyran-4- yl)methanone |
| 8 | 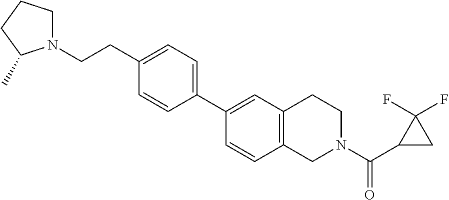 | (2,2-difluorocyclopropyl)(6- (4-(2-((R)-2- methylpyrrolidin-1- yl)ethyl)phenyl)-3,4- dihydroisoquinolin-2(1H)- yl)methanone |
| 9 | 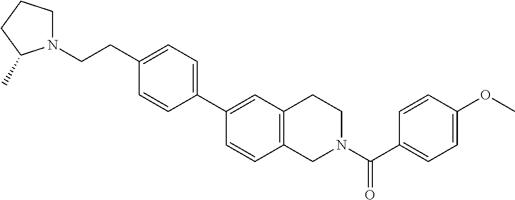 | (R)-(4-methoxyphenyl)(6-(4- (2-(2-methylpyrrolidin-1- yl)ethyl)phenyl)-3,4- dihydroisoquinolin-2(1H)- yl)methanone |
| 10 | 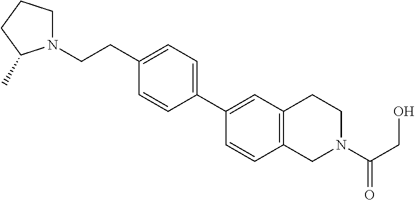 | (R)-2-hydroxy-1-(6-(4-(2-(2- methylpyrrolidin-1- yl)ethyl)phenyl)-3,4- dihydroisoquinolin-2(1H)- yl)ethanone |
| 11 | 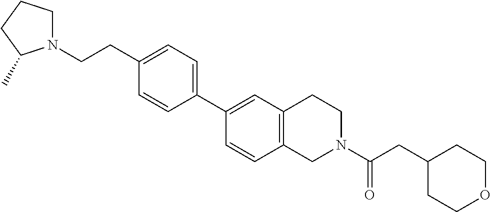 | (R)-1-(6-(4-(2-(2- methylpyrrolidin-1- yl)ethyl)phenyl)-3,4- dihydroisoquinolin-2(1H)- yl)-2-(tetrahydro-2H-pyran- 4-yl)ethanone |
| 12 | 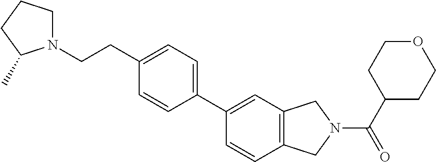 | (R)-(5-(4-(2-(2- methylpyrrolidin-1- yl)ethyl)phenyl)isoindolin-2- yl)(tetrahydro-2H-pyran-4- yl)methanone |
| 13 | 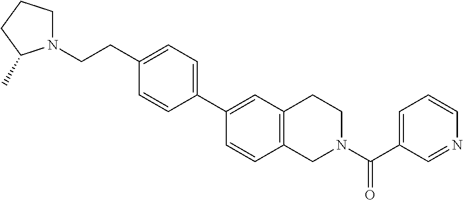 | (R)-(6-(4-(2-(2- methylpyrrolidin-1- yl)ethyl)phenyl)-3,4- dihydroisoquinolin-2(1H)- yl)(pyridin-3-yl)methanone |
| 14 | 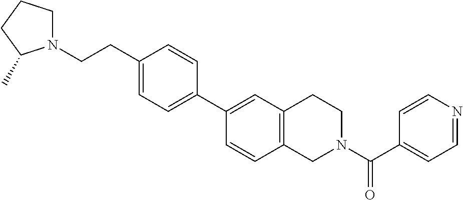 | (R)-(6-(4-(2-(2- methylpyrrolidin-1- yl)ethyl)phenyl)-3,4- dihydroisoquinolin-2(1H)- yl)(pyridin-4-yl)methanone |
| 15 | 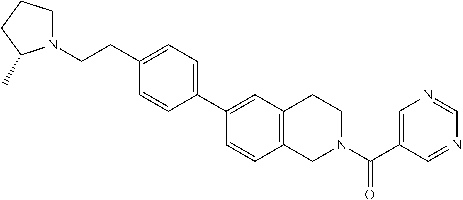 | (R)-(6-(4-(2-(2- methylpyrrolidin-1- yl)ethyl)phenyl)-3,4- dihydroisoquinolin-2(1H)- yl)(pyrimidin-5- yl)methanone |
| 16 | 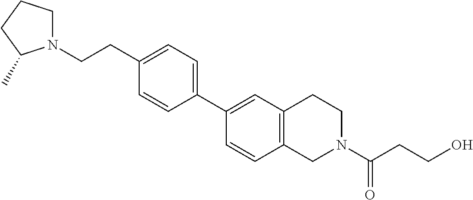 | (R)-3-hydroxy-1-(6-(4-(2-(2- methylpyrrolidin-1- yl)ethyl)phenyl)-3,4- dihydroisoquinolin-2(1H)- yl)propan-1-one |
| 17 | 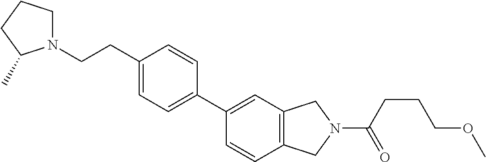 | (R)-4-methoxy-1-(5-(4-(2- (2-methylpyrrolidin-1- yl)ethyl)phenyl)isoindolin-2- yl)butan-1-one |
| 18 | 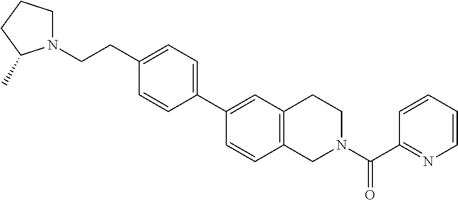 | (R)-(6-(4-(2-(2- methylpyrrolidin-1- yl)ethyl)phenyl)-3,4- dihydroisoquinolin-2(1H)- yl)(pyridin-2-yl)methanone |
| 19 | 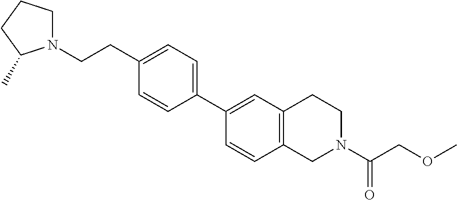 | (R)-2-methoxy-1-(6-(4-(2- (2-methylpyrrolidin-1- yl)ethyl)phenyl)-3,4- dihydroisoquinolin-2(1H)- yl)ethanone |
| 20 | 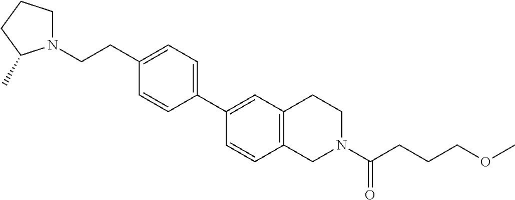 | (R)-4-methoxy-1-(6-(4-(2- (2-methylpyrrolidin-1- yl)ethyl)phenyl)-3,4- dihydroisoquinolin-2(1H)- yl)butan-1-one |
| 21 | 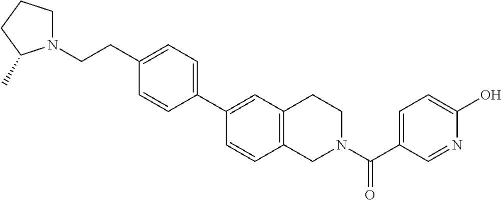 | (R)-(6-hydroxypyridin-3- yl)(6-(4-(2-(2- methylpyrrolidin-1- yl)ethyl)phenyl)-3,4- dihydroisoquinolin-2(1H)- yl)methanone |
| 22 | 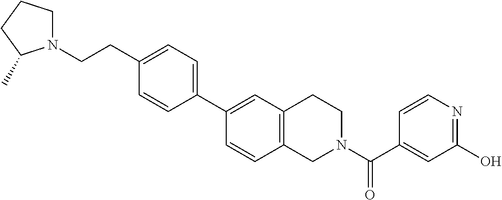 | (R)-(2-hydroxypyridin-4- yl)(6-(4-(2-(2- methylpyrrolidin-1- yl)ethyl)phenyl)-3,4- dihydroisoquinolin-2(1H)- yl)methanone |
| 23 | 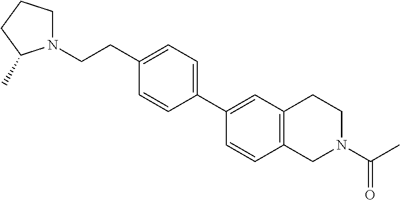 | (R)-1-(6-(4-(2-(2- methylpyrrolidin-1- yl)ethyl)phenyl)-3,4- dihydroisoquinolin-2(1H)- yl)ethanone |

TABLE B

| Cmpd No. | Chemical Structure | Chemical Name |
|---|---|---|
| 24 | 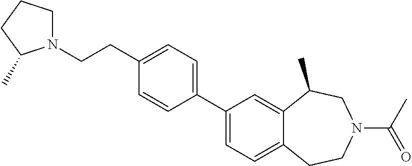 | 1-((R)-1-methyl-8-(4-(2- ((R)-2-methylpyrrolidin-1- yl)ethyl)phenyl)-4,5- dihydro-1H-benzo[d]azepin- 3(2H)-yl)ethanone |
| 25 | 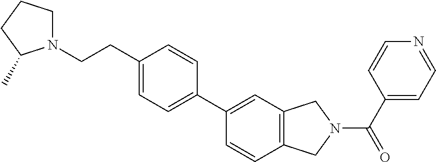 | (R)-(5-(4-(2-(2- methylpyrrolidin-1- yl)ethyl)phenyl)isoindolin-2- yl)(pyridin-4-yl)methanone |
| 26 | 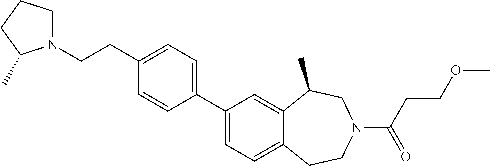 | 3-methoxy-1-((R)-1-methyl- 8-(4-(2-((R)-2- methylpyrrolidin-1- yl)ethyl)phenyl)-4,5- dihydro-1H-benzo[d]azepin- 3(2H)-yl)propan-1-one |
| 27 | 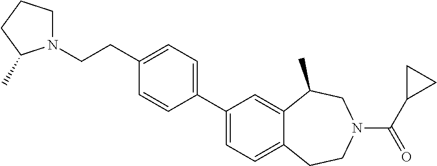 | cyclopropyl((R)-1-methyl-8- (4-(2-((R)-2- methylpyrrolidin-1- yl)ethyl)phenyl)-4,5- dihydro-1H-benzo[d]azepin- 3(2H)-yl)methanone |
| 28 | 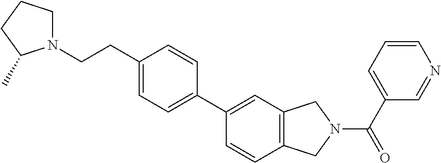 | (R)-(5-(4-(2-(2- methylpyrrolidin-1- yl)ethyl)phenyl)isoindolin-2- yl)(pyridin-3-yl)methanone |
| 29 | 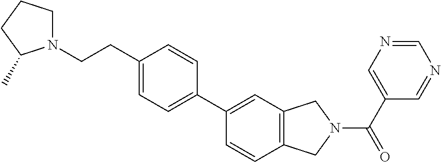 | (R)-(5-(4-(2-(2- methylpyrrolidin-1- yl)ethyl)phenyl)isoindolin-2- yl)(pyrimidin-5- yl)methanone |
| 30 | 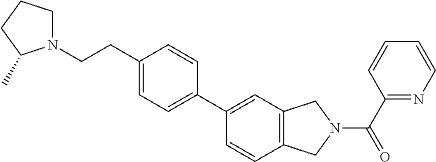 | (R)-(5-(4-(2-(2- methylpyrrolidin-1- yl)ethyl)phenyl)isoindolin-2- yl)(pyridin-2-yl)methanone |
| 31 | 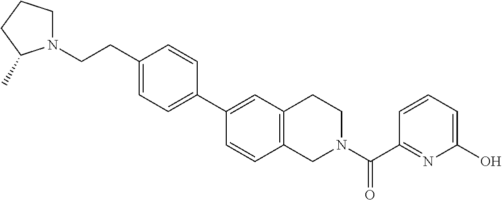 | (R)-(6-hydroxypyridin-2- yl)(6-(4-(2-(2- methylpyrrolidin-1- yl)ethyl)phenyl)-3,4- dihydroisoquinolin-2(1H)- yl)methanone |
| 32 | 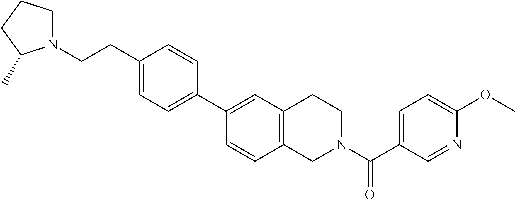 | (R)-(6-methoxypyridin-3- yl)(6-(4-(2-(2- methylpyrrolidin-1- yl)ethyl)phenyl)-3,4- dihydroisoquinolin-2(1H)- yl)methanone |
| 33 | 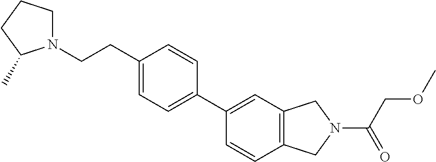 | (R)-2-methoxy-1-(5-(4-(2- (2-methylpyrrolidin-1- yl)ethyl)phenyl)isoindolin-2- yl)ethanone |
| 34 | 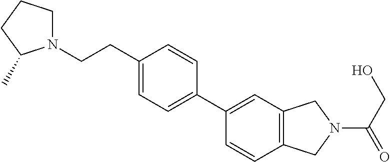 | (R)-2-hydroxy-1-(5-(4-(2-(2- methylpyrrolidin-1- yl)ethyl)phenyl)isoindolin-2- yl)ethanone |
| 35 | 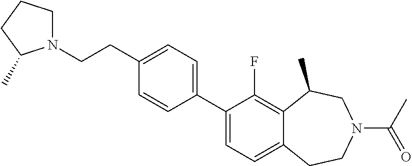 | 1-((R)-9-fluoro-1-methyl-8- (4-(2-((R)-2- methylpyrrolidin-1- yl)ethyl)phenyl)-4,5- dihydro-1H-benzo[d]azepin- 3(2H)-yl)ethanone |
| 36 | 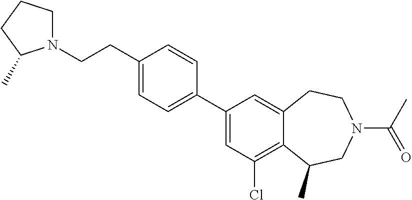 | 1-((S)-9-chloro-1-methyl-7- (4-(2-((R)-2- methylpyrrolidin-1- yl)ethyl)phenyl)-4,5- dihydro-1H-benzo[d]azepin- 3(2H)-yl)ethanone |
| 37 | 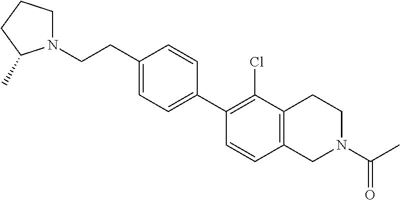 | (R)-1-(5-chloro-6-(4-(2-(2- methylpyrrolidin-1- yl)ethyl)phenyl)-3,4- dihydroisoquinolin-2(1H)- yl)ethanone |
| 38 | 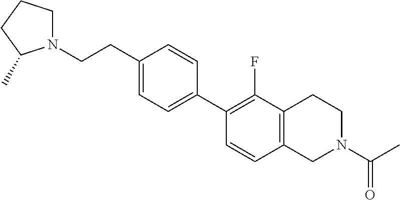 | (R)-1-(5-fluoro-6-(4-(2-(2- methylpyrrolidin-1- yl)ethyl)phenyl)-3,4- dihydroisoquinolin-2(1H)- yl)ethanone |
| 39 | 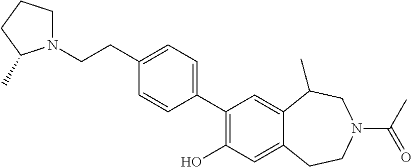 | 1-(7-hydroxy-1-methyl-8-(4- (2-((R)-2-methylpyrrolidin- 1-yl)ethyl)phenyl)-4,5- dihydro-1H-benzo[d]azepin- 3(2H)-yl)ethanone |
| 40 | 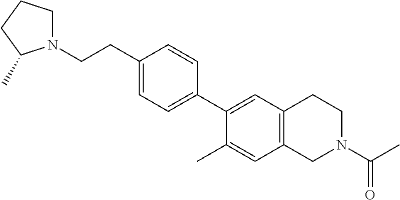 | (R)-1-(7-methyl-6-(4-(2-(2- methylpyrrolidin-1- yl)ethyl)phenyl)-3,4- dihydroisoquinolin-2(1H)- yl)ethanone |
| 41 | 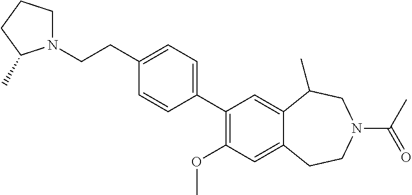 | 1-(7-methoxy-1-methyl-8- (4-(2-((R)-2- methylpyrrolidin-1- yl)ethyl)phenyl)-4,5- dihydro-1H-benzo[d]azepin- 3(2H)-yl)ethanone |
| 42 | 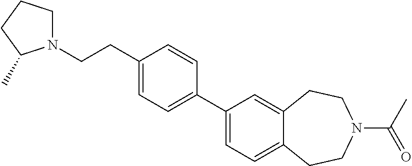 | (R)-1-(7-(4-(2-(2- methylpyrrolidin-1- yl)ethyl)phenyl)-4,5- dihydro-1H-benzo[d]azepin- 3(2H)-yl)ethanone |
| 43 | 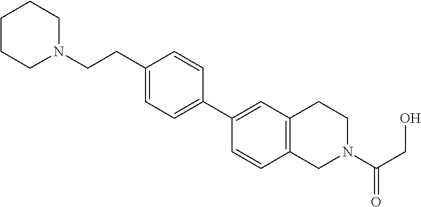 | 2-hydroxy-1-(6-(4-(2- (piperidin-1- yl)ethyl)phenyl)-3,4- dihydroisoquinolin-2(1H)- yl)ethanone |
| 44 | 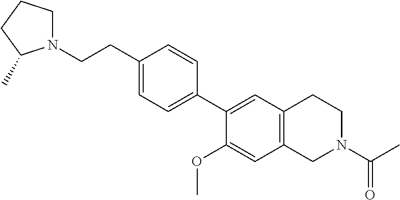 | (R)-1-(7-methoxy-6-(4-(2- (2-methylpyrrolidin-1- yl)ethyl)phenyl)-3,4- dihydroisoquinolin-2(1H)- yl)ethanone |
| 45 | 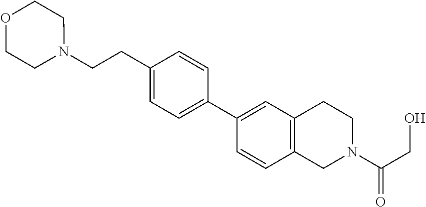 | 2-hydroxy-1-(6-(4-(2- morpholinoethyl)phenyl)- 3,4-dihydroisoquinolin- 2(1H)-yl)ethanone |
| 46 | 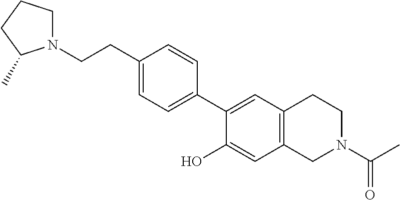 | (R)-1-(7-hydroxy-6-(4-(2-(2- methylpyrrolidin-1- yl)ethyl)phenyl)-3,4- dihydroisoquinolin-2(1H)- yl)ethanone |
| 47 | 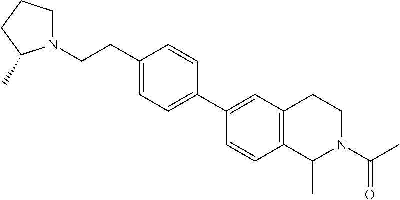 | 1-(1-methyl-6-(4-(2-((R)-2- methylpyrrolidin-1- yl)ethyl)phenyl)-3,4- dihydroisoquinolin-2(1H)- yl)ethanone |
| 48 | 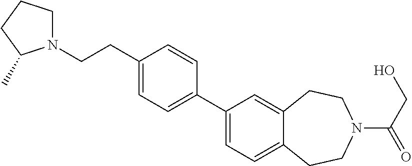 | (R)-2-hydroxy-1-(7-(4-(2-(2- methylpyrrolidin-1- yl)ethyl)phenyl)-4,5- dihydro-1H-benzo[d]azepin- 3(2H)-yl)ethanone |
| 49 | 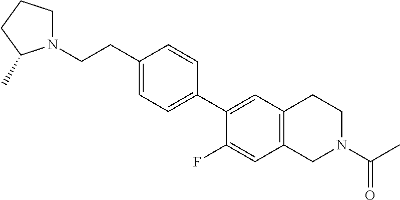 | (R)-1-(7-fluoro-6-(4-(2-(2- methylpyrrolidin-1- yl)ethyl)phenyl)-3,4- dihydroisoquinolin-2(1H)- yl)ethanone |
| 50 | 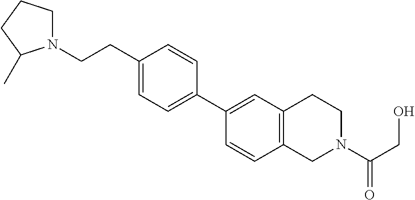 | 2-hydroxy-1-(6-(4-(2-(2- methylpyrrolidin-1- yl)ethyl)phenyl)-3,4- dihydroisoquinolin-2(1H)- yl)ethanone |
| 51 | 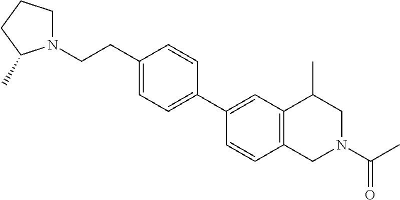 | 1-(4-methyl-6-(4-(2-((R)-2- methylpyrrolidin-1- yl)ethyl)phenyl)-3,4- dihydroisoquinolin-2(1H)- yl)ethanone |
| 52 | 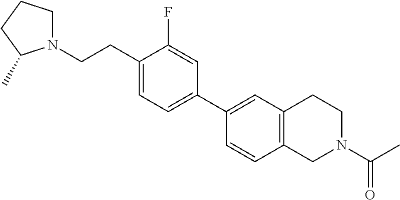 | (R)-1-(6-(3-fluoro-4-(2-(2- methylpyrrolidin-1- yl)ethyl)phenyl)-3,4- dihydroisoquinolin-2(1H)- yl)ethanone |
| 53 | 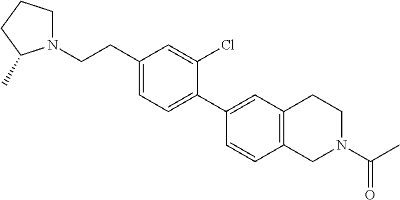 | (R)-1-(6-(2-chloro-4-(2-(2- methylpyrrolidin-1- yl)ethyl)phenyl)-3,4- dihydroisoquinolin-2(1H)- yl)ethanone |

Additionally, individual compounds and chemical genera of the present invention, for example those compounds found in TABLE A and TABLE B including diastereoisomers and enantiomers thereof, encompass all pharmaceutically acceptable salts, solvates and particularly hydrates, thereof.

The compounds of the Formula (Ia) of the present invention may be prepared according to relevant published literature procedures that are used by one skilled in the art. Exemplary reagents and procedures for these reactions appear hereinafter in the working Examples. Protection and deprotection may be carried out by procedures generally known in the art (see, for example, Greene, T. W. and Wuts, P. G. M., Protecting Groups in Organic Synthesis, 3^{rd} Edition, 1999 [Wiley]; incorporated herein by reference in its entirety).

It is understood that the present invention embraces each diastereoisomer, each enantiomer and mixtures thereof of each compound and generic formulae disclosed herein just as if they were each individually disclosed with the specific stereochemical designation for each chiral carbon. Separation of the individual isomers (such as, by chiral HPLC, recrystallization of diastereoisomeric mixtures and the like) or selective synthesis (such as, by enantiomeric selective syntheses and the like) of the individual isomers is accomplished by application of various methods which are well known to practitioners in the art.

Indications and Methods of Prophylaxis and/or Treatment

Histamine [2-(imidazol-4-yl)ethylamine] exerts its physiological effects through four distinct G-protein coupled receptors (GPCRs), termed H1, H2, H3 and H4. The histamine H3 receptor was first identified in 1983, when it was determined that the H3 receptor acted as an autoreceptor controlling both the synthesis and release of histamine (see: Arrang et al. Nature 1983, 302, 832-7). At least four human and three rat splice variants have proven functional activity in pharmacological assays (Passani et al., Trends in Pharmacol. Sci. 2004, 25, 618-625). Rat and human histamine H3 receptors also show constitutive activity which means that they can transduce a signal even in the absence of a ligand. Histamine H3 receptors also function as heteroceptors, modulating the release of a number of other transmitter substances including serotonin, acetylcholine, dopamine and noradrenaline (see: Brown et al. Prog. Neurobiol. 2001, 63, 637-672). Thus, there are a number of therapeutic applications for ligands which target the histamine H3 receptor, where the ligand functions as either an antagonist or inverse agonist (for reviews see: Leurs et al. Nat. Rev. Drug. Discov. 2005, 4, 107-120; Passani et al. Trends Pharmacol. Sci. 2004, 25, 618-625).

Accordingly, preclinical studies have identified a number of indications which are amenable to treatment with histamine H3 receptor antagonists and inverse agonists, such as compounds of the present invention. The compounds disclosed herein are believed to be useful in the treatment and/or prevention of several diseases and disorders, and in the amelioration of symptoms thereof. These compounds can be used alone or in combination with other compounds for the treatment and/or prevention of diseases and disorders. Without limitation, these diseases and disorders include the following.

Histamine H3 receptor antagonists have been shown to increase wakefulness (e.g. Lin J. S. et al. Brain Research 1990, 523, 325-330). This effect demonstrates that H3 receptor antagonists can be useful for disorders of sleep and wakefulness (Parmentier et al. J. Neurosci. 2002, 22, 7695-7711; Ligneau et al. J. Pharmacol. Exp. Ther. 1998, 287, 658-666). For example, histamine H3 receptor antagonists and inverse agonists can be used to treat the somnolence syndrome associated with different pathological conditions, for example, sleep apnea and Parkinson's disease or circumstances associated with lifestyle, for example, daytime somnolence from sleep deprivation as a result of nocturnal jobs, overwork, or jet-lag (see Passani et al., Trends Pharmacol. Sci. 2004, 25, 618-625). Somnolence is one of the major problems of public health because of its high prevalence (19-37% of the general population) and risk for causing work and traffic accidents.

Sleep apnea (alternatively sleep apnoea) is a common sleep disorder characterized by brief interruptions of breathing during sleep. These episodes, called apneas, last 10 seconds or more and occur repeatedly throughout the night. People with sleep apnea partially awaken as they struggle to breathe, but in the morning they may not be aware of the disturbances in their sleep. The most common type of sleep apnea is obstructive sleep apnea (OSA), caused by relaxation of soft tissue in the back of the throat that blocks the passage of air. Central sleep apnea (CSA) is caused by irregularities in the brain's normal signals to breathe. The hallmark symptom of the disorder is excessive daytime sleepiness. Additional symptoms of sleep apnea include restless sleep, loud snoring (with periods of silence followed by gasps), falling asleep during the day, morning headaches, trouble concentrating, irritability, forgetfulness, mood or behaviour changes, weight gain, increased heart rate, anxiety, and depression.

Few drug-based treatments of obstructive sleep apnea are known despite over two decades of research and tests. Oral administration of the methylxanthine theophylline (chemically similar to caffeine) can reduce the number of episodes of apnea, but can also produce side effects such as palpitations and insomnia. Theophylline is generally ineffective in adults with OSA, but is sometimes used to treat CSA, and infants and children with apnea. In 2003 and 2004, some neuroactive drugs, particularly modern-generation antidepressants including mirtazapine, have been reported to reduce incidences of obstructive sleep apnea. When other treatments do not completely treat the OSA, drugs are sometimes prescribed to treat a patient's daytime sleepiness or somnolence. These range from stimulants such as amphetamines to modern anti-narcoleptic medicines. The drug modafinil is seeing increased use in this role as of 2004.

In addition, for example, histamine H3 receptor antagonists and inverse agonists can be used to treat narcolepsy (Tedford et al. Soc. Neurosci. Abstr. 1999, 25, 460.3). Narcolepsy is a neurological condition most often characterized by Excessive Daytime Sleepiness (EDS), episodes of sleep and disorder of REM or rapid eye movement sleep. The main characteristic of narcolepsy is overwhelming Excessive Daytime Sleepiness (EDS), even after adequate nighttime sleep. A person with narcolepsy is likely to become drowsy or to fall asleep, often at inappropriate times and places. In addition, nighttime sleep may be fragmented with frequent wakenings. Classic symptoms of narcolepsy include, for example, cataplexy which is sudden episodes of loss of muscle function, ranging from slight weakness (such as limpness at the neck or knees, sagging facial muscles, or inability to speak clearly) to complete body collapse. Episodes may be triggered by sudden emotional reactions such as laughter, anger, surprise, or fear, and may last from a few seconds to several minutes. Another symptom of narcolepsy is sleep paralysis, which is the temporary inability to talk or move when waking up. Other symptoms include, for example, hypnagogic hallucinations which are vivid, often frightening, dream-like experiences that occur while dozing, falling asleep and/or while awakening, and automatic behaviour which occurs when a person continues to function (talking, putting things away, etc.) during sleep episodes, but awakens with no memory of performing such activities. Daytime sleepiness, sleep paralysis, and hypnagogic hallucinations also occur in people who do not have narcolepsy, such as in people who are suffering from extreme lack of sleep. Cataplexy is generally considered unique to narcolepsy.

Currently the treatments available for narcolepsy treat the symptoms, but not the underlying cause. For cataplexy and REM-sleep symptoms, antidepressant medications and other drugs that suppress REM sleep are prescribed. The drowsiness is normally treated using stimulants such as methylphenidate (Ritalin), amphetamines (Adderall), dextroamphetamine (Dexedrine), methamphetamine (Desoxyn), modafinil (Provigil), etc. Other medications used are codeine and selegiline. The cataplexy is treated using clomipramine, imipramine, or protriptyline but this need only be done in severe cases. The drug gamma-hydroxybutyrate (GHB) (Xyrem) is approved in the USA by the Food and Drug Administration to treat both the cataplexy and excessive daytime sleepiness associated with narcolepsy.

Interestingly, modafinil (Provigil) has recently been shown to increase hypothalamic histamine release (Ishizuka et al. Neurosci. Lett. 2003, 339, 143-146).

In addition, recent studies using the classic Doberman model of narcolepsy with a non-imidazole histamine H3 receptor antagonist showed that a histamine H3 receptor antagonist can reduce the number of cataplectic attacks and the duration of the attacks (Carruthers Ann. Meet. Eur. Histamine Res. Soc. 2004, Abs. p 31).

In summary, histamine H13 receptor antagonists and inverse agonists can be used for the treatment and/or prevention of conditions associated with excessive daytime sleepiness such as hypersomnia, narcolepsy, sleep apnea, time zone change disorder, and other disorders which are associated with excessive daytime sleepiness such as fibromyalgia, and multiple sclerosis (Parmentier et al., J. Neurosci. 2002, 22, 7695-7711; Ligneau et al. J. Pharmacol. Exp. Ther. 1998, 287, 658-666). Other conditions include excessive sleepiness due to shift-work, medical disorders, psychiatric disorders, narcolepsy, primary hypersomnia, and the like. Histamine H3 receptor antagonists and inverse agonists can also be used occasionally to promote wakefulness or vigilance in shift workers, sleep deprivation, post anesthesia grogginess, drowsiness as a side effect from a medication, military use and the like.

In addition, wakefulness is a prerequisite for several brain functions including attention, learning, and memory and is required for appropriate behaviours in response to environmental challenges. Histamine H3 receptor antagonists and inverse agonists have been shown to improve cognitive performance in various animal models (Hancock and Fox in Milestones in Drug Therapy, ed. Buccafusco, 2003). These compounds can be used as pro-cognitive agents and can increase vigilance. Therefore, histamine H3 receptor antagonists and inverse agonists can be used in aging or degenerative disorders in which vigilance, attention and memory are impaired, for example, as in Alzheimer's disease or other dementias.

Alzheimer's disease (AD), a neurodegenerative disorder, is the most common cause of dementia. It is characterized clinically by progressive cognitive deterioration together with neuropsychiatric symptoms and behavioural changes. The most striking early symptom is memory loss, which usually manifests as minor forgetfulness that becomes steadily more pronounced with illness progression, with relative preservation of older memories. As the disorder progresses, cognitive (intellectual) impairment extends to the domains of language, skilled movements, recognition and functions closely related to the frontal and temporal lobes of the brain such as decision-making and planning. There is currently no cure for AD, although there are drugs which offer symptomatic benefit, specifically with respect to short-term memory impairment. These drugs include acetylcholinesterase inhibitors such as donepezil (Aricept), galantamine (Razadyne) and rivastigmine (Exelon) and NMDA antagonists such as memantine.

Histamine H3 receptor antagonists and inverse agonists can be used to treat or prevent cognitive disorders (Passani et al. Trends Pharmacol. Sci. 2004, 25, 618-625), epilepsy (Vohora et al. Pharmacol. Biochem. Behav. 2001, 68, 735-741), depression (Perez-Garcia et al. Psychopharmacol. 1999, 142, 215-220), attention deficit hyperactivity disorder (ADHD), (Fox et al. Behav. Brain Res. 2002, 131, 151-61), and schizophrenia (Fox et al. J. Pharmacol. Exp. Ther. 2005, 313, 176-190). These indications are described briefly below. For additional information, see reviews by Leurs et al., Nat. Rev. Drug. Discov. 2005, 4, 107-120, and Vohora Investigational Drugs 2004, 7, 667-673). Histamine H3 receptor antagonists or inverse agonists can also be used as a novel therapeutic approach to restore cortical activation in comatose or brain-traumatized patients (Passani et al., Trends in Pharmacol. Sci. 2004, 25, 618-625).

As stated above, histamine H3 receptor antagonists and inverse agonists can be used to treat or prevent epilepsy. Epilepsy (often referred to as a seizure disorder) is a chronic neurological condition characterized by recurrent unprovoked seizures. In terms of their pattern of activity, seizures may be described as either partial (focal) or generalized. Partial seizures only involve a localized part of the brain, whereas generalized seizures involve the entire cortex. There are many different epilepsy syndromes, each presenting with its own unique combination of seizure type, typical age of onset, EEG findings, treatment, and prognosis. Some common seizure syndromes include, for example, infantile spasms (West syndrome), childhood absence epilepsy, and benign focal epilepsy of childhood (Benign Rolandic epilepsy), juvenile myoclonic epilepsy, temporal lobe epilepsy, frontal lobe epilepsy and Lennox-Gastaut syndrome.

Compounds of the present invention can be used in combination with various known drugs. For example, compounds of the present invention can be used with one or more drugs that prevent seizures or reduce seizure frequency: these include carbamazepine (common brand name Tegretol), clobazam (Frisium), clonazepam (Klonopin), ethosuximide (Zarontin), felbamate (Felbatol), fosphenyloin (Cerebyx), flurazepam (Dalmane), gabapentin (Neurontin), lamotrigine (Lamictal), levetiracetam (Keppra), oxcarbazepine (Trileptal), mephenyloin (Mesantoin), phenobarbital (Luminal), phenyloin (Dilantin), pregabalin (Lyrica), primidone (Mysoline), sodium valproate (Epilim), tiagabine (Gabitril), topiramate (Topamax), valproate semisodium (Depakote), valproic acid (Depakene, Convulex), and vigabatrin (Sabril). Other drugs are commonly used to abort an active seizure or interrupt a seizure flurry; these include diazepam (Valium) and lorazepam (Ativan). Drugs used only in the treatment of refractory status epilepticus include paraldehyde (Paral) and pentobarbital (Nembutal).

As stated above, a histamine H3 receptor antagonist or inverse agonist can be used as the sole agent of treatment or can be used in combination with other agents. For example, Vohora et al. show that a histamine H3 receptor antagonist can work as an anti-epilepsy, anti-seizure drug and also showed effect with sub-effective doses of the H3 receptor antagonist in combination with sub-effective doses of known anti-epileptic drugs (Vohora et al. Pharmacol. Biochem. Behav. 2001, 68, 735-741).

Perez-Garcia et al. (Psychopharmacol. 1999, 142, 215-220) tested the ability of a histamine H3 receptor agonist and antagonist on experimental mouse models of anxiety (elevated plus-maze) and depression (forced swimming test). They found that while the compounds did not have a significant effect on the model of anxiety, an H3 receptor antagonist did have a significant dose-dependent effect in the model of depression. Thus, histamine H3 receptor antagonists or inverse agonists can have antidepressant effects.

Clinical depression is a state of sadness or melancholia that has advanced to the point of being disruptive to an individual's social functioning and/or activities of daily living. Clinical depression affects about 16% of the population on at least one occasion in their lives. Clinical depression is currently the leading cause of disability in the U.S. as well as other countries, and is expected to become the second leading cause of disability worldwide (after heart disease) by the year 2020, according to the World Health Organization.

Compounds of the present invention can be used in combination with various known drugs. For example, compounds of the present invention can be used with one or more of the drugs currently available that can relieve the symptoms of depression. They include, for example, monoamine oxidase inhibitors (MAOIs) such as Nardil or Moclobemide (Manerix), tricyclic antidepressants, selective serotonin reuptake inhibitors (SSRIs) such as fluoxetine (Prozac), paroxetine (Paxil), escitalopram (Lexapro), and sertraline (Zoloft), norepinephrine reuptake inhibitors such as reboxetine (Edronax), and serotonin-norepinephrine reuptake inhibitors (SNRIs) such as venlafaxine (Effexor) and duloxetine (Cymbalta).

As stated above, histamine H3 receptor antagonists and inverse agonists can be used to treat or prevent attention deficit hyperactivity disorder (ADHD). According to the Diagnostic and Statistical Manual of Mental Disorders-IV-TR, ADHD is a developmental disorder that arises in childhood, in most cases before the age of 7 years, is characterized by developmentally inappropriate levels of inattention and/or hyperactive-impulsive behavior, and results in impairment in one or more major life activities, such as family, peer, educational, occupational, social, or adaptive functioning. ADHD can also be diagnosed in adulthood.

The first-line medications used to treat ADHD are mostly stimulants, which work by stimulating the areas of the brain responsible for focus, attention, and impulse control. The use of stimulants to treat a syndrome often characterized by hyperactivity is sometimes referred to as a paradoxical effect, but there is no real paradox in that stimulants activate brain inhibitory and self-organizing mechanisms permitting the individual to have greater self-regulation. The stimulants used include, for example, methylphenidate (sold as Ritalin, Ritalin SR and Ritalin LA), Metadate, Metadate ER, Metadate CD, Concerta, Focalin, Focalin XR or Methylin. The stimulants also include, for example, amphetamines such dextroamphetamine, sold as Dexedrine, Dexedrine Spansules, Adderall, and Adderall XR, a trade name for a mixture of dextroamphetamine and laevoamphetamine salts, methamphetamine sold as Desoxyn, bupropion, a dopamine and norepinephrine reuptake inhibitor, marketed under the brand name Wellbutrin. A non-stimulant medication to treat ADHD is Atomoxetine (sold as Strattera) a norepinephrine reuptake inhibitor. Other drugs sometimes used for ADHD include, for example, benzphetamine, Provigil/Alertec/modafinil and clonidine. Recently it has been reported that in a rat pup model for ADHD, a histamine H3 receptor antagonist was at least as effective as methylphenidate (Ritalin) (Hancock and Fox in Milestones in Drug Therapy, ed. Buccafusco, 2003). Compounds of the present invention can be used in combination with various known drugs. For example, compounds of the present invention can be used with one or more of the drugs used to treat ADHD and related disorders.

As stated above, histamine H3 receptor antagonists and inverse agonists can be used to treat or prevent schizophrenia. Schizophrenia is a psychiatric diagnosis that describes a mental disorder characterized by impairments in the perception or expression of reality and by significant social or occupational dysfunction. A person experiencing untreated schizophrenia is typically characterized as demonstrating disorganized thinking, and as experiencing delusions or auditory hallucinations. Although the disorder is primarily thought to affect cognition, it can also contribute to chronic problems with behavior and emotion. Schizophrenia is often described in terms of “positive” and “negative” symptoms. Positive symptoms include delusions, auditory hallucinations and thought disorder, and are typically regarded as manifestations of psychosis. Negative symptoms are so named because they are considered to be the loss or absence of normal traits or abilities, and include features such as flat, blunted or constricted affect and emotion, poverty of speech and lack of motivation. Some models of schizophrenia include formal thought disorder and planning difficulties in a third group, a “disorganization syndrome.”

The first line pharmacological therapy for schizophrenia is usually the use of antipsychotic medication. Antipsychotic drugs are only thought to provide symptomatic relief from the positive symptoms of psychosis. The newer atypical antipsychotic medications (such as clozapine, risperidone, olanzapine, quetiapine, ziprasidone and aripiprazole) are usually preferred over older typical antipsychotic medications (such as chlorpromazine and haloperidol) due to their favorable side-effect profile. While the atypical antipsychotics are associated with less extra pyramidal side-effects and tardive dyskinesia than the conventional antipsychotics, some of the agents in this class (especially olanzapine and clozapine) appear to be associated with metabolic side effects such as weight gain, hyperglycemia and hypertriglyceridemia that must be considered when choosing appropriate pharmacotherapy.

Histamine H3 receptor antagonists or inverse agonists can be used to treat obesity (Hancock, Curr. Opin. Investig. Drugs 2003, 4, 1190-1197). The role of neuronal histamine in food intake has been established for many years and neuronal histamine release and/or signalling has been implicated in the anorectic actions of known mediators in the feeding cycle such as leptin, amylin and bombesin. In the brain, the H3 receptor is implicated in the regulation of histamine release in the hypothalamus. Moreover, in situ hybridization studies have revealed histamine H3 receptor mRNA expression in rat brown adipose tissue, indicating a role in the regulation of thermogenesis (Karlstedt et al., Mol. Cell. Neurosci. 2003, 24, 614-622). Furthermore, histamine H3 receptor antagonists have been investigated in various preclinical models of obesity and have shown to be effective in reducing food intake, reducing weight, and decreasing total body fat in mice (Hancock, et al. Eur. J. Pharmacol. 2004, 487, 183-197). The most common drugs used for the treatment of obesity are sibutramine (Meridia) and orlistat (Xenical), both of which have limited effectiveness and significant side effects. Therefore, novel anti-obesity agents, such as histamine H3 receptor antagonists or inverse agonists, are needed.

Histamine H3 receptor antagonists or inverse agonists can also be used to treat upper airway allergic responses (U.S. Pat. Nos. 5,217,986; 5,352,707 and 5,869,479) including allergic rhinitis and nasal congestion. Allergic rhinitis is a frequently occurring chronic disease that affects a large number of people. Recent analysis of histamine H3 receptor expression in the periphery by quantitative PCR revealed that H3 receptor mRNA is abundantly expressed in human nasal mucosa (Varty et al. Eur. J. Pharmacol. 2004, 484, 83-89). In addition, in a cat model of nasal decongestion, a combination of histamine H3 receptor antagonists with the H1 receptor antagonist chlorpheniramine resulted in significant nasal decongestion without the hypertensive effect seen with adrenergic agonists. (McLeod et al. Am. J. Rhinol. 1999, 13, 391-399). Thus, histamine H3 receptor antagonists or inverse agonists can be used alone or in combination with H1 receptor blockage for the treatment of allergic rhinitis and nasal congestion.

Histamine H3 receptor antagonists or inverse agonists have therapeutic potential for the treatment of pain (Medhurst et al. Biochemical Pharmacology (2007), 73(8), 1182-1194).

### Pharmaceutical Compositions

A further aspect of the present invention pertains to pharmaceutical compositions comprising one or more compounds as described herein and one or more pharmaceutically acceptable carriers. Some embodiments pertain to pharmaceutical compositions comprising a compound of the present invention and a pharmaceutically acceptable carrier.

Some embodiments of the present invention include a method of producing a pharmaceutical composition comprising admixing at least one compound according to any of the compound embodiments disclosed herein and a pharmaceutically acceptable carrier.

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound of the invention in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20^{th} Edition, 2000, Lippincott Williams & Wilkins, (Editors: Gennaro et al.)

While it is possible that, for use in the prophylaxis or treatment, a compound of the invention may, in an alternative use, be administered as a raw or pure chemical, it is preferable however to present the compound or active ingredient as a pharmaceutical formulation or composition further comprising a pharmaceutically acceptable carrier.

The invention thus further provides pharmaceutical formulations comprising a compound of the invention or a pharmaceutically acceptable salt, solvate, hydrate or derivative thereof together with one or more pharmaceutically acceptable carriers thereof and/or prophylactic ingredients. The carrier(s) must be “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, subcutaneous and intravenous) administration or in a form suitable for administration by inhalation, insufflation or by a transdermal patch. Transdermal patches dispense a drug at a controlled rate by presenting the drug for absorption in an efficient manner with a minimum of degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds of the invention, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds of the present invention or a solvate or physiologically functional derivative thereof can be used as active ingredients in pharmaceutical compositions, specifically as histamine H3 receptor modulators. By the term “active ingredient” is defined in the context of a “pharmaceutical composition” and is intended to mean a component of a pharmaceutical composition that provides the primary pharmacological effect, as opposed to an “inactive ingredient” which would generally be recognized as providing no pharmaceutical benefit.

The dose when using the compounds of the present invention can vary within wide limits and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds of the present invention. Representative doses of the present invention include, but not limited to, about 0.001 mg to about 5000 mg, about 0.001 mg to about 2500 mg, about 0.001 mg to about 1000 mg, 0.001 mg to about 500 mg, 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3 or 4 doses. Depending on the individual and as deemed appropriate from the patient's physician or caregiver it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds of the present invention and as part of a drug combination. The dosage regimen for treating a disease condition with the compounds and/or compositions of this invention is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods of this invention.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations. The daily dose can be divided, especially when relatively large amounts are administered as deemed appropriate, into several, for example 2, 3 or 4 part administrations. If appropriate, depending on individual behavior, it may be necessary to deviate upward or downward from the daily dose indicated.

The compounds of the present invention can be administrated in a wide variety of oral and parenteral dosage forms. It will be obvious to those skilled in the art that the following dosage forms may comprise, as the active component, either a compound of the invention or a pharmaceutically acceptable salt, solvate or hydrate of a compound of the invention.

For preparing pharmaceutical compositions from the compounds of the present invention, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is mixed with the carrier having the necessary binding capacity in suitable proportions and compacted to the desire shape and size. The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams or sprays containing in addition to the active ingredient such carriers as are known in the art to be appropriate.

Liquid form preparations include solutions, suspensions and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds according to the invention may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds of the present invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds of the present invention as an aerosol can be prepared by processes well known to the person skilled in the art. For their preparation, for example, solutions or dispersions of the compounds of the present invention in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds according to the invention may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like, such as those pharmaceutically acceptable salts listed in Journal of Pharmaceutical Sciences, 66:1-19 (1977), incorporated herein by reference in its entirety.

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds of this invention may form solvates with standard low molecular weight solvents using methods known to the skilled artisan.

Compounds of the present invention can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds of the invention containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987, both of which are hereby incorporated by reference in their entirety.

Some embodiments of the present invention include a method of producing a pharmaceutical composition for “combination-therapy” comprising admixing at least one compound according to any of the compound embodiments disclosed herein, together with at least one known pharmaceutical agent as described herein and a pharmaceutically acceptable carrier.

It is noted that when the histamine H3 receptor modulators are utilized as active ingredients in a pharmaceutical composition, these are not intended for use only in humans, but in other non-human mammals as well. Indeed, recent advances in the area of animal health-care mandate that consideration be given for the use of active agents, such as histamine H3 receptor modulators, for the treatment of an H3-associated disease or disorder in companionship animals (e.g., cats, dogs, etc.) and in livestock animals (e.g., cows, chickens, fish, etc.) Those of ordinary skill in the art are readily credited with understanding the utility of such compounds in such settings.

### Hydrates and Solvates

It is understood that when the phrase “pharmaceutically acceptable salts, solvates and hydrates” is used when referring to a particular formula herein, it is intended to embrace solvates and/or hydrates of compounds of the particular formula, pharmaceutically acceptable salts of compounds of the particular formula as well as solvates and/or hydrates of pharmaceutically acceptable salts of compounds of the particular formula.

The compounds of the present invention can be administrated in a wide variety of oral and parenteral dosage forms. It will be apparent to those skilled in the art that the following dosage forms may comprise, as the active component, either a compound of the invention or a pharmaceutically acceptable salt or as a solvate or hydrate thereof. Moreover, various hydrates and solvates of the compounds of the invention and their salts will find use as intermediates in the manufacture of pharmaceutical compositions. Typical procedures for making and identifying suitable hydrates and solvates, outside those mentioned herein, are well known to those in the art; see for example, pages 202-209 of K. J. Guillory, “Generation of Polymorphs, Hydrates, Solvates, and Amorphous Solids,” in: Polymorphism in Pharmaceutical Solids, ed. Harry G. Brittan, Vol. 95, Marcel Dekker, Inc., New York, 1999, incorporated herein by reference in its entirety. Accordingly, one aspect of the present invention pertains to hydrates and solvates of compounds of the present invention and/or their pharmaceutical acceptable salts, as described herein, that can be isolated and characterized by methods known in the art, such as, thermogravimetric analysis (TGA), TGA-mass spectroscopy, TGA-Infrared spectroscopy, powder X-ray diffraction (PXRD), Karl Fisher titration, high resolution X-ray diffraction, and the like. There are several commercial entities that provide quick and efficient services for identifying solvates and hydrates on a routine basis. Example companies offering these services include Wilmington PharmaTech (Wilmington, Del.), Avantium Technologies (Amsterdam) and Aptuit (Greenwich, Conn.).

### Crystalline Forms

A further aspect of the present invention pertains to a crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride (the HCl salt of Compound 10). Form 1 of the HCl salt of compound 10 can be identified by its unique solid state signature with respect to, for example, differential scanning calorimetry (DSC), X-ray powder diffraction (PXRD), and other solid state methods. Further characterization with respect to water or solvent content of the crystalline form can be gauged by any of the following methods for example, thermogravimetric analysis (TGA), DSC and the like. For DSC, it is known that the temperatures observed will depend upon the rate of temperature change as well as sample preparation technique and the particular instrument employed. Thus, the values reported herein relating to DSC thermograms can vary by plus or minus about 4° C. The values reported herein relating to DSC thermograms can also vary by plus or minus about 20 joules per gram. For PXRD, the relative intensities of the peaks can vary, depending upon the sample preparation technique, the sample mounting procedure and the particular instrument employed. Moreover, instrument variation and other factors can often affect the 2θ values. Therefore, the peak assignments of diffraction patterns can vary by plus or minus about 0.2°2θ. For TGA, the features reported herein can vary by about ±5° C. The TGA features reported herein can also vary by about ±2% weight change due to, for example, sample variation. Further characterization with respect to hygroscopicity of the crystalline form can be gauged by, for example, dynamic vapor sorption (DVS). The DVS features reported herein can vary by about ±5% relative humidity. The DVS features reported herein can also vary and by about ±5% weight change. The physical properties of Form 1 of the HCl salt of Compound 10 are summarized in Table 1 below.

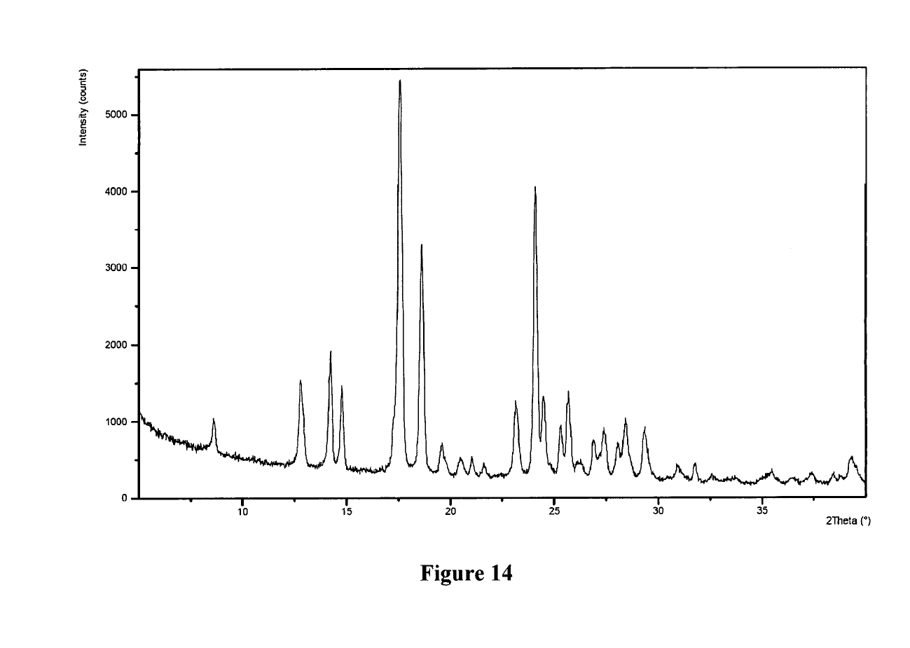

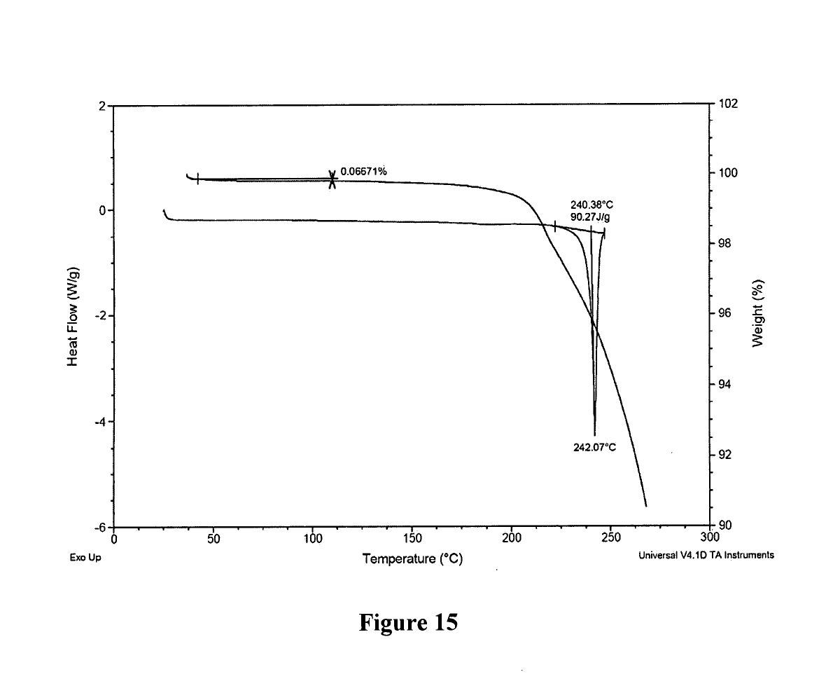

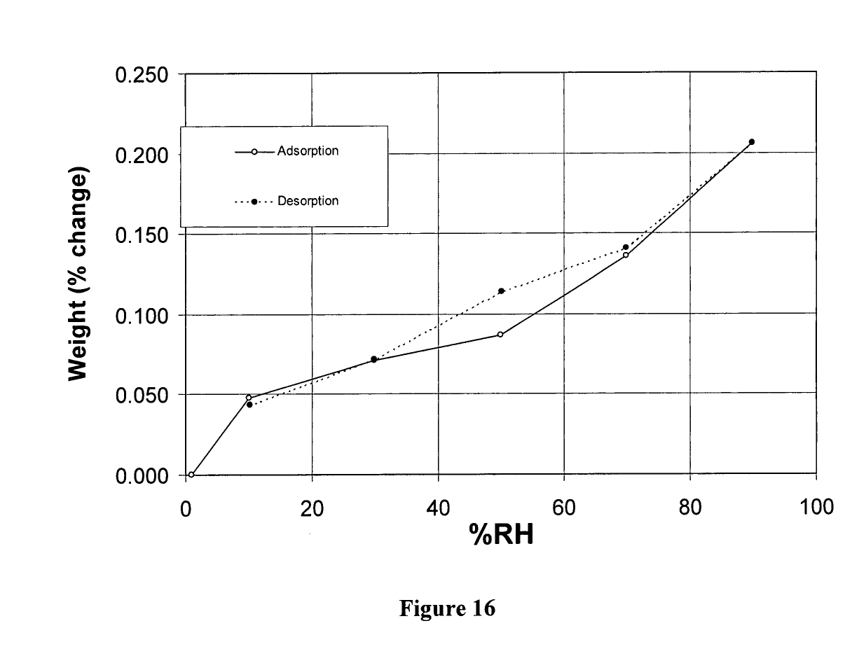

TABLE 1
Compound 10 HCl Salt (Form 1)

| | |
|---|---|
| TGA | FIG. 15: <0.7% weight loss below |
| | about 110° C. |
| DSC | FIG. 15: extrapolated onset |
| | temperature: 240° C.; endotherm peak |
| | temperature: 242° C.; associated heat |
| | flow 90 J/g |
| PXRD | FIG. 14: Peaks of ≧8% relative |
| | intensity at 12.8, 14.2, 14.8, 17.2, 17.6, |
| | 18.6, 23.1, 23.2, 24.1, 24.5, 25.3, 25.6, |
| | 25.7, 26.8, 26.9, 27.4, 28.1, 28.3, 28.4 |
| | and 29.3 °2θ |
| DVS | FIG. 16: absorption of less than 0.25% |
| | at 90% relative humidity |

The small weight loss observed in the TGA data suggests that Form 1 of the HCl salt of Compound 10 is an anhydrous, non-solvated crystalline form. The DSC thermogram further reveals a melting endotherm with an onset at about 240° C.

DVS data for the crystalline form of the Form 1 of the HCl salt of Compound 10 reveals low hygroscopicity, with absorption of about 0.25% at 90% relative humidity.

Certain X-ray powder diffraction peaks for Form 1 of the HCl salt of Compound 10 are shown in Table 2 below.

TABLE 2
HCl salt of Compound 10 (Form 1)
PXRD Peaks with Relative Intensity of 8% or Higher

| Peak Position (°2θ) | Relative Intensity (%) |
|---|---|
| 12.8 | 21.75 |
| 14.2 | 29.2 |
| 14.8 | 20.71 |
| 17.2 | 12.29 |
| 17.6 | 100 |
| 18.6 | 57.84 |
| 23.1 | 16.33 |
| 23.2 | 17.41 |
| 24.1 | 73.64 |
| 24.5 | 20.47 |
| 25.3 | 13.06 |
| 25.6 | 17.71 |
| 25.7 | 21.92 |
| 26.8 | 9.01 |
| 26.9 | 9.07 |
| 27.4 | 13.17 |
| 28.1 | 8.5 |
| 28.3 | 11.98 |
| 28.4 | 14.37 |
| 29.3 | 12.16 |

One aspect of the present invention is directed to a crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride having an X-ray powder diffraction pattern comprising a peak, in terms of 2θ, at about 17.6°. In some embodiments, the crystalline form has an X-ray powder diffraction pattern comprising a peak, in terms of 2θ, at about 24.1°. In some embodiments, the crystalline form has an X-ray powder diffraction pattern comprising a peak, in terms of 2θ, at about 17.6° and about 18.6°. In some embodiments, the crystalline form has an X-ray powder diffraction pattern comprising a peak, in terms of 2θ, at about 24.1° and about 18.6°. In some embodiments, the crystalline form has an X-ray powder diffraction pattern comprising a peak, in terms of 2θ, at about 17.6°, about 24.1° and about 18.6°. In some embodiments, the crystalline form has an X-ray powder diffraction pattern comprising a peak, in terms of 2θ, at about 17.6°, about 24.1°, about 18.6°, about 14.2°, about 25.7°, about 12.8°, and about 14.8°. In some embodiments, the crystalline form has an X-ray powder diffraction pattern comprising a peak, in terms of 2θ, at about 17.6°, about 24.1°, about 18.6°, about 14.2°, about 25.7°, about 12.8°, about 14.8°, about 24.5°, about 25.6°, about 23.2° and about 23.1°. In yet further embodiments, the crystalline form has an X-ray powder diffraction pattern substantially as shown in FIG. 14, wherein by “substantially” is meant that the reported peaks can vary by about ±0.2°2θ and also that the relative intensities of the reported peaks can vary.

In some embodiments, the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride has a differential scanning calorimetry thermogram comprising an endotherm with an extrapolated onset temperature between about 230° C. and about 250° C. In some embodiments, the crystalline form has a differential scanning calorimetry thermogram comprising an endotherm with an extrapolated onset temperature at about 240° C. In some embodiments, the crystalline form has a differential scanning calorimetry thermogram comprising an endotherm with a peak temperature between about 232° C. and about 252° C. In some embodiments, the crystalline form has a differential scanning calorimetry thermogram comprising an endotherm with a peak temperature at about 242° C. In some embodiments, the crystalline form has a differential scanning calorimetry thermogram comprising an endotherm with an associated heat flow of about 90 joules per gram. In further embodiments, the crystalline form has a differential scanning calorimetry thermogram substantially as shown in FIG. 15, wherein by “substantially” is meant that the reported DSC features can vary by about ±4° C. and also that the reported DSC features can vary by about ±20 joules per gram.

In some embodiments, the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride has a dynamic vapor sorption profile substantially as shown in FIG. 16, wherein by “substantially” is meant that the reported DVS features can vary by about ±5% relative humidity and also that the reported DVS features can vary by about ±5% weight change.

In some embodiments, the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride has a thermogravimetric analysis profile substantially as shown in FIG. 15, wherein by “substantially” is meant that the reported TGA features can vary by about ±5° C. and also that the reported TGA features can vary by about ±2% weight change.

The crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride, the HCl salt of Compound 10, described herein can be prepared by any of the suitable procedures known in the art for preparing crystalline polymorphs. In some embodiments Form 1 of the HCl salt of Compound 10 can be prepared as described in Example 1.57. In some embodiments, Form 1 of the HCl salt of Compound 10 can be prepared by heating crystalline HCl salt of Compound 10, containing one or more crystalline forms other than Form 1. In some embodiments, Form 1 of the HCl salt of Compound 10 can be prepared by recrystallizing crystalline HCl salt of Compound 10, containing one or more crystalline forms other than Form 1 of the HCl salt of Compound 10.

### Other Utilities

Another object of the present invention relates to radio-labeled compounds of the present invention that would be useful not only in radio-imaging but also in assays, both in vitro and in vivo, for localizing and quantitating the histamine H3 receptor in tissue samples, including human and for identifying histamine H3 receptor ligands by inhibition binding of a radio-labeled compound. It is a further object of this invention to develop novel H3 receptor assays of which comprise such radio-labeled compounds.

The present invention embraces isotopically-labeled compounds of the present invention. Isotopically or radio-labeled compounds are those which are identical to compounds disclosed herein, but for the fact that one or more atoms are replaced or substituted by an atom having an atomic mass or mass number different from the atomic mass or mass number most commonly found in nature. Suitable radionuclides that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁷⁵Br, ⁷⁶Br, ⁷⁷Br, ⁸²Br, ¹²³I, ¹²⁴I, ¹²⁵I and ¹³¹I. The radionuclide that is incorporated in the instant radio-labeled compounds will depend on the specific application of that radio-labeled compound. For example, for in vitro histamine H3 receptor labeling and competition assays, compounds that incorporate ³H, ¹⁴C, ⁸²Br, ¹²⁵I, ¹³¹I or ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labeled” or “labeled compound” is a compound of Formula (Ia), (Ic) or (Ie) that has incorporated at least one radionuclide; in some embodiments the radionuclide is selected from the group consisting of ³H, ¹⁴C, ¹²⁵I, ³⁵S and ⁸²Br.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the scarcer radio-isotope or nonradioactive isotope.

Synthetic methods for incorporating radio-isotopes into organic compounds are applicable to compounds of the invention and are well known in the art. These synthetic methods, for example, incorporating activity levels of tritium into target molecules, are as follows:

A. Catalytic Reduction with Tritium Gas: This procedure normally yields high specific activity products and requires halogenated or unsaturated precursors.

B. Reduction with Sodium Borohydride [³H]: This procedure is rather inexpensive and requires precursors containing reducible functional groups such as aldehydes, ketones, lactones, esters and the like.

C. Reduction with Lithium Aluminum Hydride [³H]: This procedure offers products at almost theoretical specific activities. It also requires precursors containing reducible functional groups such as aldehydes, ketones, lactones, esters and the like.

D. Tritium Gas Exposure Labeling: This procedure involves exposing precursors containing exchangeable protons to tritium gas in the presence of a suitable catalyst.

E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:

A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A represented procedure was reported by Zhu, G-D. and co-workers in J. Org. Chem., 2002, 67, 943-948.

B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.

C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled histamine H3 receptor compound of Formula (Ia) can be used in a screening assay to identify/evaluate compounds. In general terms, a newly synthesized or identified compound (i.e., test compound) can be evaluated for its ability to reduce binding of the “radio-labeled compound of Formula (Ia)” to the H3 receptor. Accordingly, the ability of a test compound to compete with the “radio-labeled compound of Formula (Ia)” for the binding to the histamine H3 receptor directly correlates to its binding affinity.

The labeled compounds of the present invention bind to the histamine H3 receptor. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM and in still yet another embodiment the labeled inhibitor has an IC₅₀ less than about 0.1 μM.

Other uses of the disclosed receptors and methods will become apparent to those in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1

### Syntheses of Compounds of the Present Invention

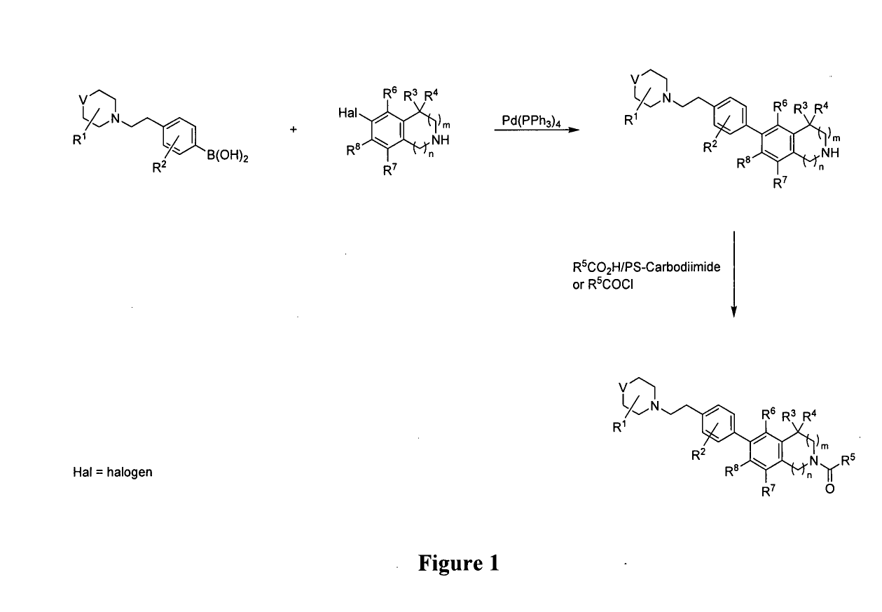

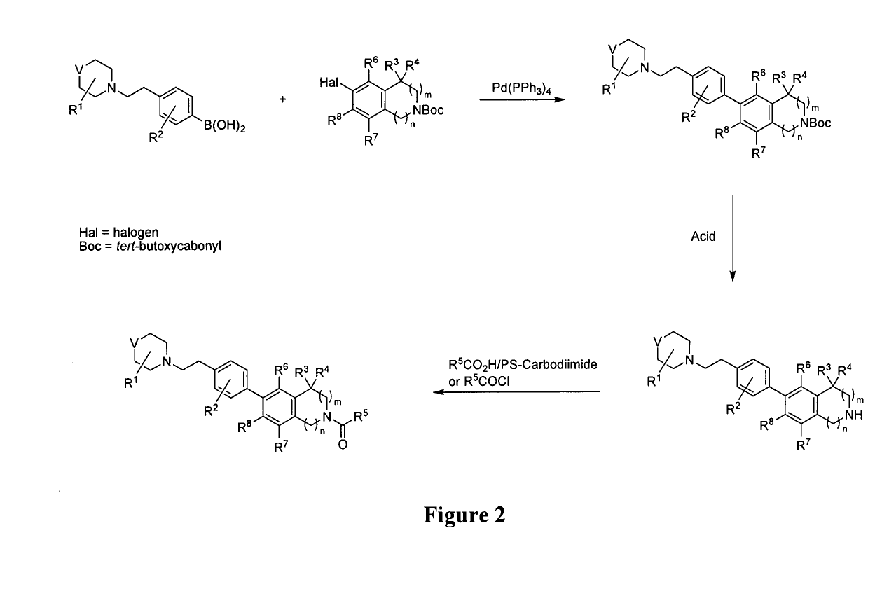

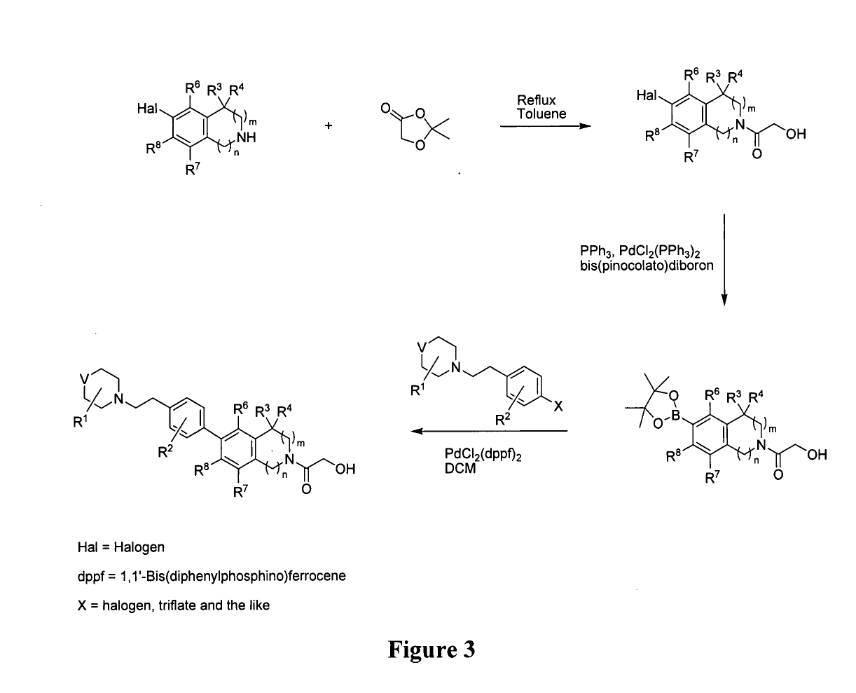

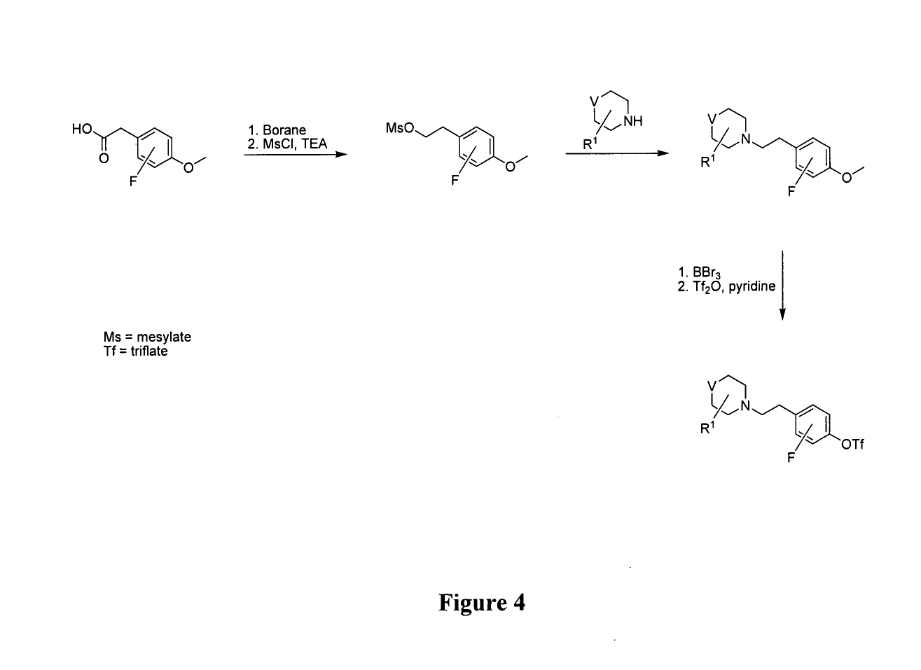

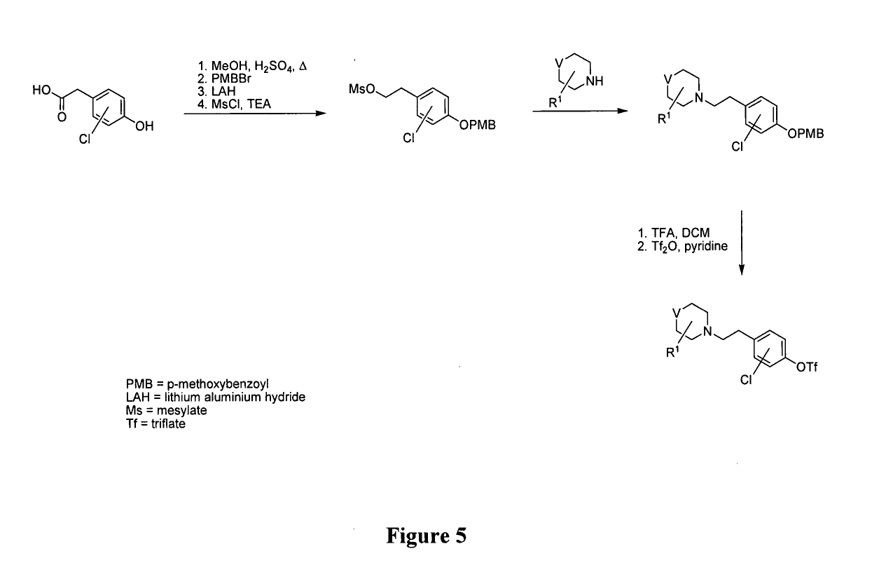

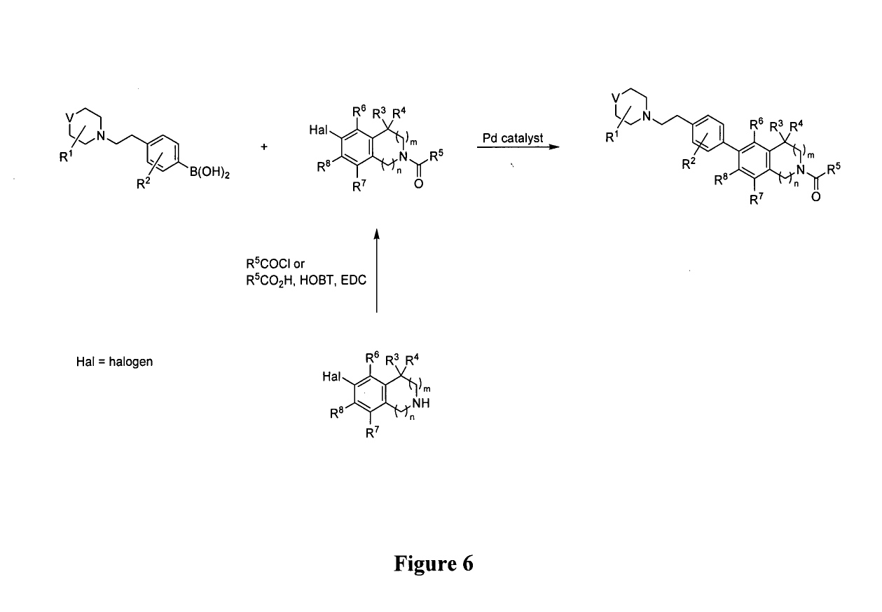

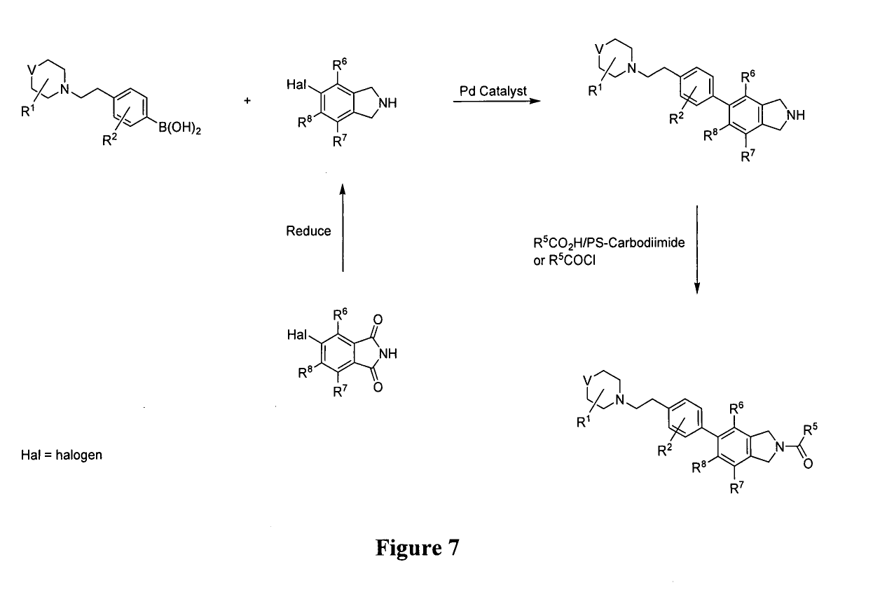

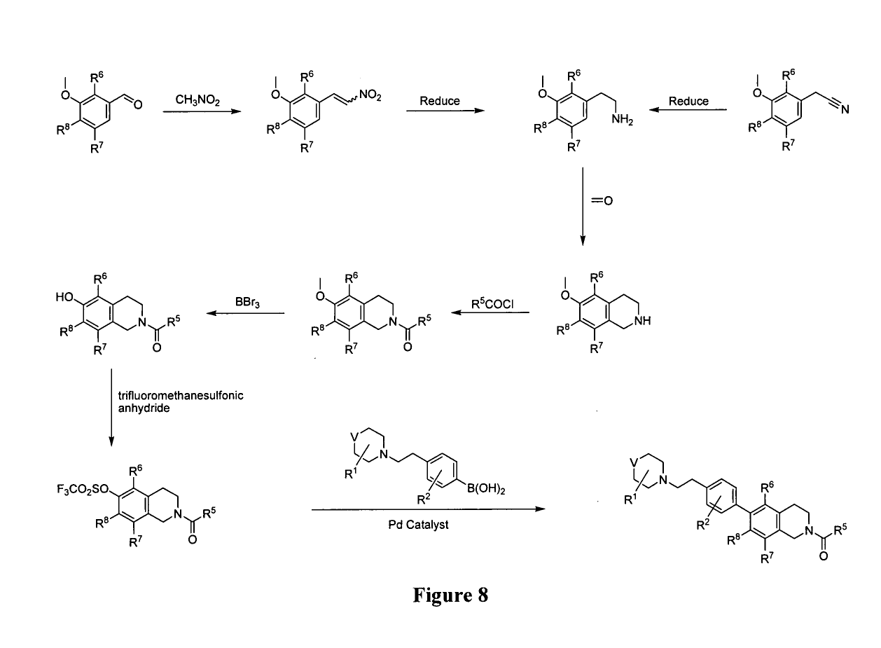

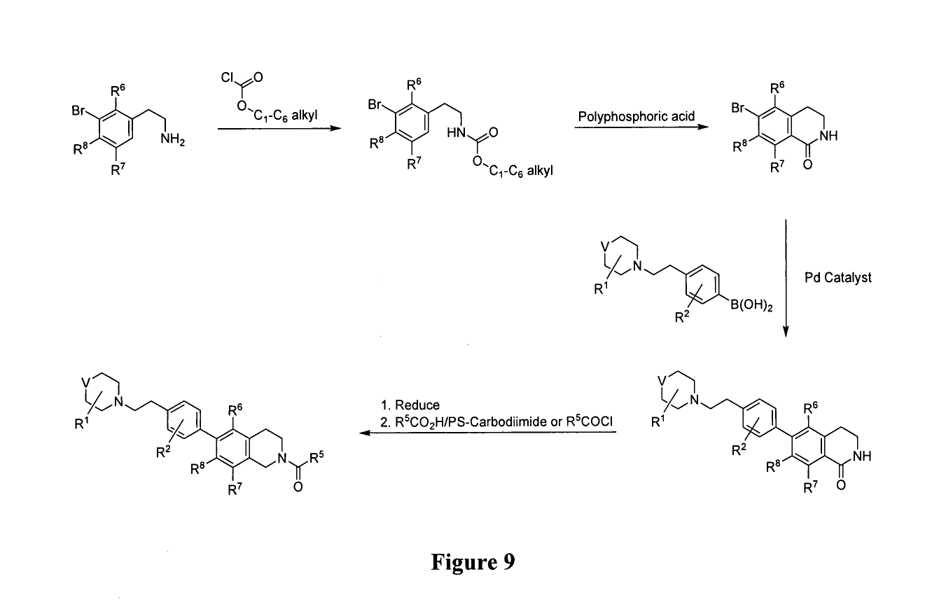

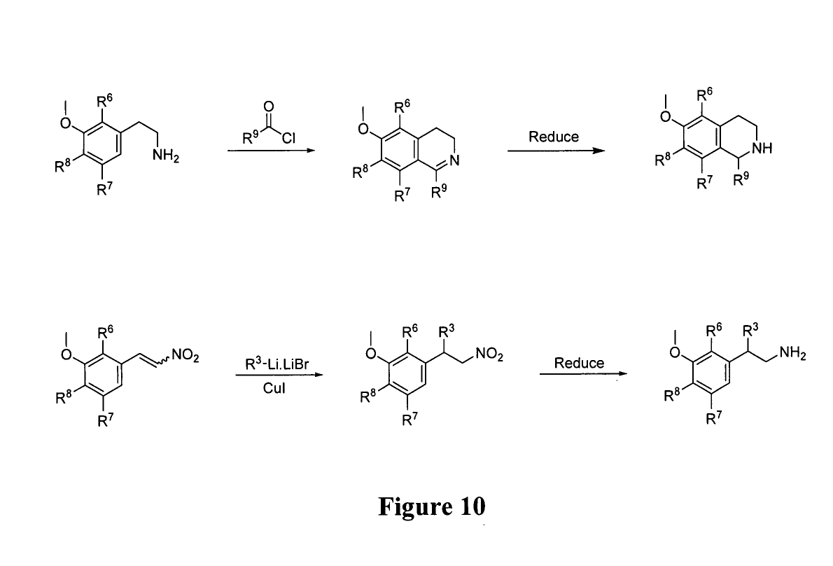

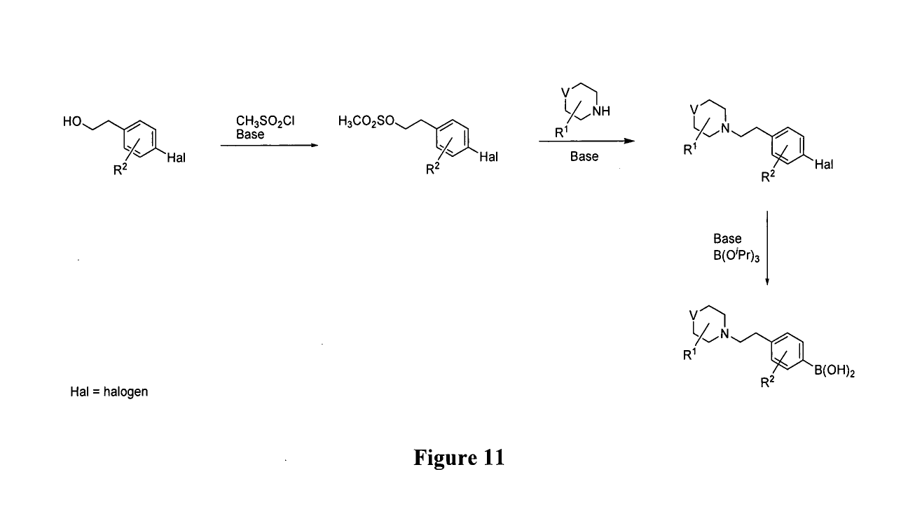

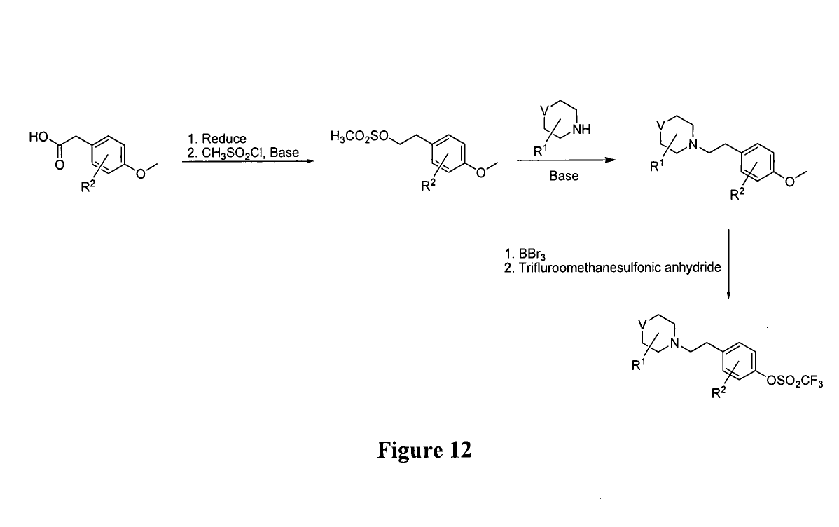

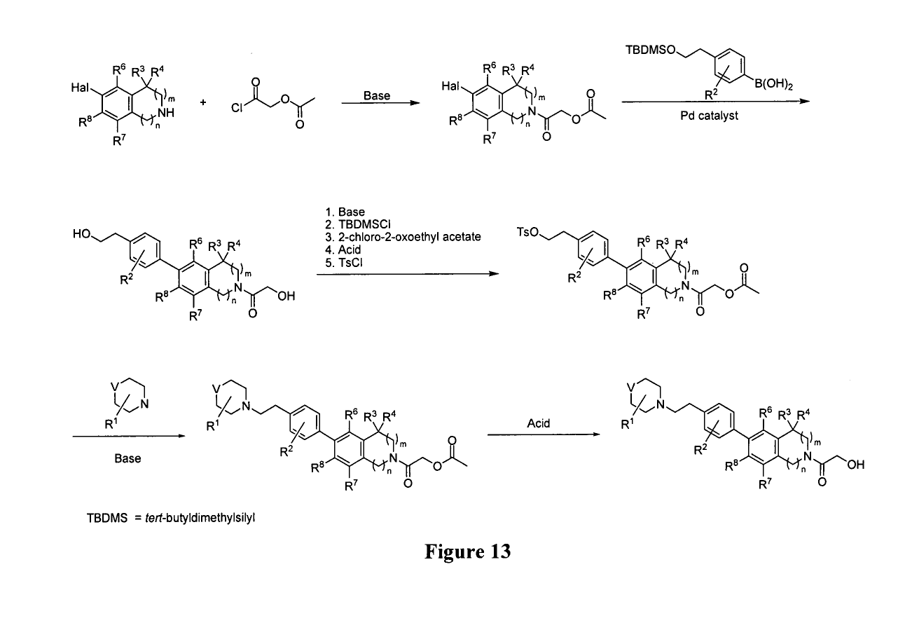

Illustrated syntheses for compounds of the present invention are shown in FIGS. 1 through 13 where the symbols have the same definitions as used throughout this disclosure.

The compounds of the invention and their syntheses are further illustrated by the following examples. The following examples are provided to further define the invention without, however, limiting the invention to the particulars of these examples. The compounds described herein, supra and infra, are named according to the CS ChemDraw Ultra Version 7.0.1, AutoNom version 2.2, or CS ChemDraw Ultra Version 9.0.7. In certain instances common names are used and it is understood that these common names would be recognized by those skilled in the art.

Chemistry: Proton nuclear magnetic resonance (¹H NMR) spectra were recorded on a Bruker Avance-400 equipped with a QNP (Quad Nucleus Probe) or a BBI (Broad Band Inverse) and z-gradient. Chemical shifts are given in parts per million (ppm) with the residual solvent signal used as reference. NMR abbreviations are used as follows: s=singlet, d=doublet, dd=doublet of doublets, ddd=doublet of doublet of doublets, dt=doublet of triplets, t=triplet, td=triplet of doublets, tt=triplet of triplets, q=quartet, m=multiplet, bs=broad singlet, bt=broad triplet. Microwave irradiations were carried out using a Smith Synthesizer™ or an Emrys Optimizer™ (Biotage). Thin-layer chromatography (TLC) was performed on silica gel 60 F₂₅₄ (Merck), preparatory thin-layer chromatography (prep TLC) was preformed on PK6F silica gel 60 A 1 mm plates (Whatman) and column chromatography was carried out on a silica gel column using Kieselgel 60, 0.063-0.200 mm (Merck). Evaporation was done under reduced pressure on a Büchi rotary evaporator.

LCMS spec: HPLC-pumps: LC-10AD VP, Shimadzu Inc.; HPLC system controller: SCL-10A VP, Shimadzu Inc; UV-Detector: SPD-10A VP, Shimadzu Inc; Autosampler: CTC HTS, PAL, Leap Scientific; Mass spectrometer: API 150EX with Turbo Ion Spray source, AB/MDS Sciex; Software: Analyst 1.2.

### Example 1.1

### Preparation of (R)-6-(4-(2-(2-Methylpyrrolidin-1-yl)ethyl)phenyl)-1,2,3,4-tetrahydroisoquinoline

To a round-bottom flask was added 6-bromo-1,2,3,4-tetrahydroisoquinoline hydrochloride (2.00 g, 8.05 mmol), (R)-4-(2-(2-methylpyrrolidin-1-yl)ethyl)phenylboronic acid (2.063 g, 8.85 mmol), tetrakis(triphenylphosphine)palladium (0) (0.279 g, 0.241 mmol), benzene (30.00 mL), ethanol (10.00 mL), and 2.0 M aqueous solution of sodium bicarbonate (8.05 mL, 16.09 mmol). The reaction mixture was refluxed for 6 h. Upon completion, water was added and the mixture was extracted with ethyl acetate. The organic layer was washed with brine, dried over Na₂SO₄, and concentrated. The residue was taken up in 1 M HCl solution and washed with ethyl acetate. The aqueous layer was basified with 10% aqueous NaOH to pH˜11, extracted with ethyl acetate, and concentrated. The residue was purified by silica gel column, eluting with 5-10% 2.0 M ammonia in methanol/DCM to give a yellow solid (1.20 g). LCMS m/z=321.4 [M+H]⁺; ¹H NMR (400 MHz, DMSO-d₆) δ ppm 0.99-1.04 (m, 3H), 1.22-1.33 (m, 1H), 1.59-1.69 (m, 2H), 1.81-1.92 (m, 1H), 2.13 (q, J=8.67 Hz, 1H), 2.20-2.34 (m, 2H), 2.65-2.83 (m, 5H), 2.94-3.04 (m, 3H), 3.10-3.18 (m, 1H), 3.91 (s, 2H), 7.09 (d, J=8.08 Hz, 1H), 7.29 (d, J=8.08 Hz, 2H), 7.33-7.40 (m, 2H), 7.53 (d, J=8.08 Hz, 2H).

### Example 1.2

### Preparation of (R)-2-Hydroxy-1-(6-(4-(2-(2-methylpyrrolidin-1-yl)ethyl)phenyl)-3,4-dihydroisoquinolin-2(1H)-yl)ethanone Hydrochloride (Compound 10)

To a solution of PS-Carbodiimide (4.68 mmol) and 2-hydroxyacetic acid (0.119 g, 1.560 mmol) in dichloromethane was added (R)-6-(4-(2-(2-methylpyrrolidin-1-yl)ethyl)phenyl)-1,2,3,4-tetrahydroisoquinoline (0.250 g, 0.780 mmol) and triethylamine (0.174 mL, 1.248 mmol). The reaction mixture was stirred overnight at room temperature. The mixture was filtered and the resin was rinsed with dichloromethane. The filtrate was concentrated and purified by preparative HPLC. The appropriate fractions were combined and basified with 10% aqueous NaOH, and extracted with ethyl acetate. The organic layers were combined, washed with brine, dried over Na₂SO₄, and concentrated. The free base obtained was converted to a hydrochloric salt using 1.0 M HCl in diethyl ether to give the title compound as an off-white powder (0.140 g). LCMS m/z=379.5 [M+H]⁺; ¹H NMR (400 MHz, CDCl₃) δ ppm 1.28 (d, J=6.7 Hz, 0.3H), 1.68 (d, J=6.6 Hz, 2.7H), 1.97-2.14 (m, 2H), 2.18-2.36 (m, 2H), 2.81-3.02 (m, 4H), 3.10-3.27 (m, 2H), 3.46-3.61 (m, 3H), 3.90-4.03 (m, 2H), 4.28 (d, J=3.03 Hz, 2H), 4.48 (s, 1H), 4.83 (s, 1H), 7.16-7.26 (m, 1H), 7.31-7.40 (m, 3H), 7.43 (dd, J=8.08, 1.52 Hz, 1H), 7.53 (d, J=8.08 Hz, 2H).

### Example 1.3

### Preparation of (2,2-Difluorocyclopropyl)(6-(4-(2-((R)-2-methylpyrrolidin-1-yl)ethyl)phenyl)-3,4-dihydroisoquinolin-2(1H)-yl)methanone Hydrochloride (Compound 8)

The title compound was prepared in a similar manner as stated above in Example 1.2. LCMS m/z=425.4 [M+H]⁺; ¹H NMR (400 MHz, CDCl₃) δ ppm 0.79-1.04 (m, 1H), 1.16-1.33 (m, 2.3H), 1.66-1.76 (m, 2.7H), 1.79-1.96 (m, 2H), 1.97-2.17 (m, 2H), 2.18-2.37 (m, 2H), 2.56-2.72 (m, 1H), 2.79-3.00 (m, 2H), 3.00-3.09 (m, 1H), 3.10-3.26 (m, 2H), 3.45-3.64 (m, 1H), 3.85-3.95 (m, 1H), 4.00 (s, 1H), 4.82 (s, 2H), 7.23 (d, J=7.83 Hz, 1H), 7.30-7.40 (m, 3H), 7.40-7.48 (m, 1H), 7.54 (d, J=6.06 Hz, 2H).

### Example 1.4

### Preparation of (R)-1-(6-(4-(2-(2-Methylpyrrolidin-1-yl)ethyl)phenyl)-3,4-dihydroisoquinolin-2(1H)-yl)-2-(tetrahydro-2H-pyran-4-yl)ethanone Hydrochloride (Compound 11)

The title compound was prepared in a similar manner as stated above in Example 1.2. LCMS m/z=447.6 [M+H]⁺.

### Example 1.5

### Preparation of (R)-(6-(4-(2-(2-Methylpyrrolidin-1-yl)ethyl)phenyl)-3,4-dihydroisoquinolin-2(1H)-yl)(pyrimidin-5-yl)methanone Hydrochloride (Compound 15)

The title compound was prepared in a similar manner as stated above in Example 1.2. LCMS m/z=427.1 [M+H]⁺; ¹H NMR (400 MHz, CDCl₃) δ ppm 1.16 (d, J=5.56 Hz, 3H), 1.50 (s, 1H), 1.70-2.04 (m, 3H), 2.21-2.49 (m, 3H), 2.84-2.96 (m, 2H), 2.97-3.18 (m, 3H), 3.33 (s, 1H), 3.72 (t, J=4.80 Hz, 1H), 4.06 (s, 1H), 4.66 (s, 1H), 4.95 (s, 1H), 7.27-7.34 (m, 3H), 7.40 (d, J=7.83 Hz, 1H), 7.45-7.55 (m, 3H), 8.89 (s, 2H), 9.32 (s, 1H).

### Example 1.6

### Preparation of (R)-(6-(4-(2-(2-Methylpyrrolidin-1-yl)ethyl)phenyl)-3,4-dihydroisoquinolin-2(1H)-yl)(pyridin-2-yl)methanone Hydrochloride (Compound 18)

The title compound was prepared in a similar manner as stated above in Example 1.2. LCMS m/z=426.3 [M+H]⁺.

### Example 1.7

### Preparation of (R)-4-Methoxy-1-(6-(4-(2-(2-methylpyrrolidin-1-yl)ethyl)phenyl)-3,4-dihydroisoquinolin-2(1H)-yl)butan-1-one Hydrochloride (Compound 20)

The title compound was prepared in a similar manner as stated above in Example 1.2. LCMS m/z=421.4 [M+H]⁺; ¹H NMR (400 MHz, CDCl₃) δ ppm 1.27 (d, J=10.86 Hz, 0.3H), 1.63-1.76 (m, 2.7H), 1.92-2.18 (m, 3H), 2.19-2.38 (m, 2H), 2.45-2.65 (m, 4H), 2.95 (s, 3H), 3.09-3.29 (m, 2H), 3.33 (s, 3H), 3.42-3.62 (m, 4H), 3.70-4.06 (m, 3H), 4.67-4.82 (m, 2H), 7.21 (s, 1H), 7.30-7.38 (m, 3H), 7.41 (d, J=7.58 Hz, 1H), 7.54 (s, 2H).

### Example 1.8

### Preparation of (R)-(6-Hydroxypyridin-3-yl)(6-(4-(2-(2-methylpyrrolidin-1-yl)ethyl)phenyl)-3,4-dihydroisoquinolin-2(1H)-yl)methanone Hydrochloride (Compound 21)

The title compound was prepared in a similar manner as stated above in Example 1.2. LCMS m/z=442.6 [M+H]⁺; ¹H NMR (400 MHz, CDCl₃) δ ppm 1.26 (d, J=5.05 Hz, 0.3H), 1.67 (d, J=6.57 Hz, 2.7H), 1.96-2.15 (m, 3H), 2.17-2.36 (m, 2H), 2.80-2.99 (m, 2H), 3.02 (t, J=5.81 Hz, 2H), 3.09-3.27 (m, 2H), 3.43-3.59 (m, 2H), 3.86 (s, 2H), 3.96-4.08 (m, 1H), 4.80 (s, 2H), 6.64 (d, J=9.35 Hz, 1H), 7.18 (d, J=7.33 Hz, 1H), 7.33 (d, J=8.08 Hz, 1H), 7.37 (s, 1H), 7.42 (d, J=7.83 Hz, 1H), 7.53 (d, J=8.08 Hz, 2H), 7.65 (dd, J=9.47, 2.40 Hz, 1H), 7.71 (d, J=2.02 Hz, 1H).

### Example 1.9

### Preparation of (R)-(2-Hydroxypyridin-4-yl)(6-(4-(2-(2-methylpyrrolidin-1-yl)ethyl)phenyl)-3,4-dihydroisoquinolin-2(1H)-yl)methanone Hydrochloride (Compound 22)

The title compound was prepared in a similar manner as stated above in Example 1.2. LCMS m/z=442.5 [M+H]⁺; ¹H NMR (400 MHz, CDCl₃) δ ppm 1.13 (t, J=5.68 Hz, 3H), 1.40-1.53 (m, 1H), 1.67-1.88 (m, 2H), 1.88-2.01 (m, 1H), 2.23 (q, J=8.84 Hz, 1H), 2.28-2.40 (m, 2H), 2.77-2.98 (m, 3H), 3.00-3.13 (m, 2H), 3.23-3.33 (m, 1H), 3.70 (t, J=5.68 Hz, 1H), 4.00 (t, J=5.94 Hz, 1H), 4.62 (s, 1H), 4.90 (s, 1H), 6.30-6.39 (m, 1H), 6.60 (s, 1H), 7.24-7.33 (m, 3H), 7.34-7.43 (m, 1H), 7.44-7.53 (m, 4H).

### Example 1.10

### Preparation of (R)-Cyclopropyl(6-(4-(2-(2-methylpyrrolidin-1-yl)ethyl)phenyl)-3,4-dihydroisoquinolin-2(1H)-yl)methanone Hydrochloride (Compound 3)

To a solution of (R)-6-(4-(2-(2-methylpyrrolidin-1-yl)ethyl)phenyl)-1,2,3,4-tetrahydroisoquinoline (0.080 g, 0.250 mmol) and triethylamine (0.104 mL, 0.749 mmol) in dichloromethane was added cyclopropanecarbonyl chloride (0.023 mL, 0.250 mmol). The reaction was stirred at ambient temperature for 20 min. The mixture was concentrated and purified by preparative HPLC. The appropriate fractions were combined, neutralized with 10% aqueous NaOH, and extracted with ethyl acetate. The organic layers were combined, washed with brine, dried over Na₂SO₄, and concentrated. The free base obtained was converted to a hydrochloric salt using 1.0 M HCl in diethyl ether to give the title compound as an off-white solid (0.062 g). LCMS m/z=389.4 [M+H]⁺; ¹H NMR (400 MHz, CDCl₃) δ ppm 0.78-0.86 (m, 2H), 1.01-1.08 (m, 2H), 1.24-1.30 (m, 1.3H), 1.65 (d, J=6.57 Hz, 2.7H), 1.79-1.89 (m, 1H), 1.96-2.13 (m, 2H), 2.18-2.34 (m, 2H), 2.81-3.05 (m, 3H), 3.06-3.29 (m, 2H), 3.38-3.49 (m, 1H), 3.52-3.62 (m, 1H), 3.84-3.91 (m, 1H), 3.91-4.06 (m, 2H), 4.77 (s, 1H), 4.91 (s, 1H), 7.22 (d, J=8.08 Hz, 1H), 7.30-7.46 (m, 4H), 7.54 (d, J=8.08 Hz, 2H).

### Example 1.11

### Preparation of (R)-(6-(4-(2-(2-Methylpyrrolidin-1-yl)ethyl)phenyl)-3,4-dihydroisoquinolin-2(1H)-yl)(tetrahydro-2H-pyran-4-yl)methanone Hydrochloride (Compound 7)

The title compound was prepared in a similar manner as stated above in Example 1.10. LCMS m/z=433.6 [M+H]⁺; ¹H NMR (400 MHz, CDCl₃) δ ppm 1.27-1.34 (m, 0.3H), 1.47 (d, J=6.57 Hz, 2.7H), 1.58-1.87 (m, 5H), 1.99-2.22 (m, 2H), 2.29-2.41 (m, 1H), 2.91 (t, J=5.94 Hz, 1H), 2.98-3.20 (m, 4H), 3.22-3.36 (m, 2H), 3.49-3.59 (m, 3H), 3.59-3.70 (m, 1H), 3.70-3.79 (m, 1H), 3.80-3.90 (m, 2H), 3.93-4.02 (m, 2H), 4.72 (s, 1H), 4.83 (s, 1H), 7.21-7.31 (m, 1H), 7.37-7.49 (m, 4H), 7.62 (d, J=8.08 Hz, 2H).

### Example 1.12

### Preparation of (R)-(4-Methoxyphenyl)(6-(4-(2-(2-methylpyrrolidin-1-yl)ethyl)phenyl)-3,4-dihydroisoquinolin-2(1H)-yl)methanone (Compound 9)

The title compound was prepared in a similar manner as stated above in Example 1.10. LCMS m/z=455.2 [M+H]⁺; ¹H NMR (400 MHz, CDCl₃) δ ppm 1.27 (bs, 0.3H), 1.56 (d, J=6.32, 3.28 Hz, 2.7H), 1.89-2.15 (m, 2H), 2.25 (s, 3H), 2.80-3.37 (m, 9H), 3.52-3.81 (m, 2H), 3.92-4.22 (m, 2H), 4.63-5.02 (m, 2H), 6.91-7.02 (m, 2H), 7.27-7.34 (m, 2H), 7.36 (s, 2H), 7.45 (dd, J=8.59, 3.28 Hz, 2H), 7.53 (d, J=6.82 Hz, 3H).

### Example 1.13

### Preparation of (R)-(6-(4-(2-(2-Methylpyrrolidin-1-yl)ethyl)phenyl)-3,4-dihydroisoquinolin-2(1H)-yl)(pyridin-3-yl)methanone (Compound 13)

The title compound was prepared in a similar manner as stated above in Example 1.10. LCMS m/z=426.3 [M+H]⁺; ¹H NMR (400 MHz, CDCl₃) δ ppm 1.29 (d, J=6.82 Hz, 0.3H), 1.56 (d, J=6.57 Hz, 2.7H), 1.86-2.13 (m, 2H), 2.18-2.36 (m, 2H), 2.87-3.14 (m, 5H), 3.17-3.38 (m, 2H), 3.56-3.80 (m, 2H), 4.06 (s, 2H), 4.66 (s, 1H), 4.96 (s, 1H), 7.24-7.62 (m, 7H), 7.71-7.86 (m, 1H), 8.24 (d, J=7.07 Hz, 1H), 8.76-9.04 (m, 2H).

### Example 1.14

### Preparation of (R)-(6-(4-(2-(2-Methylpyrrolidin-1-yl)ethyl)phenyl)-3,4-dihydroisoquinolin-2(1H)-yl)(pyridin-4-yl)methanone (Compound 14)

The title compound was prepared in a similar manner as stated above in Example 1.10. LCMS m/z=426.1 [M+H]⁺; ¹H NMR (400 MHz, CDCl₃) δ ppm 1.29 (d, J=7.07 Hz, 0.3H), 1.57 (d, J=6.57 Hz, 2.7H), 1.90-2.12 (m, 2H), 2.19-2.33 (m, 2H), 2.85-3.01 (m, 3H), 3.02-3.12 (m, 2H), 3.17-3.30 (m, 4H), 3.58-3.67 (m, 2H), 4.01-4.12 (m, 2H), 4.53 (s, 1H), 4.96 (s, 1H), 7.28-7.34 (m, 2H), 7.35-7.43 (m, 1H), 7.45-7.57 (m, 2H), 7.69 (s, 2H), 8.88 (s, 2H).

### Example 1.15

### Preparation of (R)-2-Methoxy-1-(6-(4-(2-(2-methylpyrrolidin-1-yl)ethyl)phenyl)-3,4-dihydroisoquinolin-2(1H)-yl)ethanone (Compound 19)

The title compound was prepared in a similar manner as stated above in Example 1.10. LCMS m/z=393.3 [M+H]⁺; ¹H NMR (400 MHz, CDCl₃) δ ppm 1.28 (d, J=6.57 Hz, 0.3H), 1.69 (d, J=6.32 Hz, 2.7H), 1.97-2.16 (m, 2H), 2.19-2.36 (m, 2H), 2.83-3.01 (m, 4H), 3.09-3.27 (m, 2H), 3.46 (s, 3H), 3.51-3.55 (m, 2H), 3.74 (t, J=5.68 Hz, 1H), 3.88 (t, J=5.81 Hz, 1H), 3.93-4.04 (m, 1H), 4.21 (s, 2H), 4.69 (s, 1H), 4.79 (s, 1H), 7.15-7.26 (m, 1H), 7.30-7.37 (m, 3H), 7.41 (d, J=8.08 Hz, 1H), 7.53 (d, J=7.83 Hz, 2H).

### Example 1.16

### Preparation of (R)-1-(6-(4-(2-(2-Methylpyrrolidin-1-yl)ethyl)phenyl)-3,4-dihydroisoquinolin-2(1H)-yl)ethanone Hydrochloride (Compound 23)

The title compound was prepared in a similar manner as stated above in Example 1.10. LCMS m/z=363.6 [M+H]⁺; ¹H NMR (400 MHz, CDCl₃) δ ppm 1.14 (d, J=6.06 Hz, 3H), 1.41-1.54 (m, 1H), 1.68-1.79 (m, 1H), 1.79-1.88 (m, 1H), 1.89-2.02 (m, 1H), 2.20 (d, J=3.79 Hz, 3H), 2.22-2.30 (m, 1H), 2.31-2.43 (m, 2H), 2.80-2.94 (m, 3H), 2.97 (t, J=5.81 Hz, 1H), 3.03-3.13 (m, 1H), 3.30 (t, J=7.45 Hz, 1H), 3.71 (t, J=5.94 Hz, 1H), 3.86 (t, J=5.94 Hz, 1H), 4.66 (s, 1H), 4.77 (s, 1H), 7.14-7.24 (m, 1H), 7.29 (d, J=7.07 Hz, 2H), 7.36 (d, J=8.59 Hz, 1H), 7.42 (d, J=8.08 Hz, 1H), 7.47-7.54 (m, 2H).

### Example 1.17

### Preparation of (R)-3-Hydroxy-1-(6-(4-(2-(2-methylpyrrolidin-1-yl)ethyl)phenyl)-3,4-dihydroisoquinolin-2(1H)-yl)propan-1-one (Compound 16)

To a solution of (R)-6-(4-(2-(2-methylpyrrolidin-1-yl)ethyl)phenyl)-1,2,3,4-tetrahydroisoquinoline dihydrochloride (0.030 g, 0.076 mmol) and triethylamine (0.043 mL, 0.31 mmol) in DCM was added oxetan-2-one (0.0082 g, 0.11 mmol). The reaction mixture was stirred for 8 h at ambient temperature. The mixture was concentrated and purified by preparative HPLC to give the title compound (0.007 g). LCMS m/z=393.3 [M+H]⁺; ¹H NMR (400 MHz, CDCl₃) δ ppm 1.31 (d, J=7.07 Hz, 0.3H), 1.46 (d, J=6.32 Hz, 2.7H), 1.60-1.81 (m, 2H), 1.83-1.97 (m, 1H), 2.00-2.20 (m, 2H), 2.27-2.41 (m, 2H), 2.62-2.72 (m, 2H), 2.92-3.02 (m, 2H), 3.03-3.19 (m, 2H), 3.46-3.55 (m, 1H), 3.56-3.71 (m, 3H), 3.71-3.83 (m, 1H), 4.22-4.42 (m, 3H), 4.53 (s, 1H), 7.29 (d, J=7.83 Hz, 1H), 7.41 (d, J=8.08 Hz, 2H), 7.50-7.57 (m, 2H), 7.62 (d, J=8.08 Hz, 2H).

### Example 1.18

### Preparation of (R)-7-(4-(2-(2-Methylpyrrolidin-1-yl)ethyl)phenyl)-1,2,3,4-tetrahydroisoquinoline Dihydrochloride

### Step A: Preparation of (R)-tert-Butyl 7-(4-(2-(2-Methylpyrrolidin-1-yl)ethyl)phenyl)-3,4-dihydroisoquinoline-2(1H)-carboxylate

To a thick-walled vial was added tert-butyl 7-bromo-3,4-dihydroisoquinoline-2(1H)-carboxylate (0.500 g, 1.60 mmol), (R)-4-(2-(2-methylpyrrolidin-1-yl)ethyl)phenylboronic acid (0.373 g, 1.60 mmol), aqueous Na₂CO₃ (2 M solution, 1.60 mL, 3.20 mmol), and Pd(PPh₃)₄ (0.055 mg, 0.048 mmol) in a mixture of EtOH (1 mL) and benzene (3 mL). The resulting reaction mixture was heated in under microwave irradiation at 100° C. for 120 min. The reaction mixture was diluted with water and the organic layer was separated. The aqueous layer was extracted with EtOAc. The combined organic phases were concentrated, dissolved in ACN/H₂O/AcOH and purified by HPLC (0.1% TFA in acetonitrile/0.1% TFA in water). The combined fractions were basified with 2 M Na₂CO₃ and extracted three times with EtOAc. The combined organics were dried over Na₂SO₄, filtered, and concentrated to provide the title compound as a clear, yellow oil (304 mg). ¹H NMR (400 MHz, Methanol-d₄) δ ppm 1.36 (d, J=6.32 Hz, 3H), 1.50 (s, 9H), 1.63-1.74 (m, 1H), 1.97-2.07 (m, 2H), 2.17-2.33 (m, 1H), 2.86 (d, J=5.05 Hz, 2H), 2.94-3.09 (m, 4H), 3.14-3.26 (m, 1H), 3.38-3.50 (m, 1H), 3.51-3.62 (m, 1H), 3.66 (s, 2H), 4.61 (s, 2H), 7.16-7.28 (m, 1H), 7.31-7.48 (m, 4H), 7.58 (d, J=8.08 Hz, 2H).

### Step B: Preparation of (R)-7-(4-(2-(2-Methylpyrrolidin-1-yl)ethyl)phenyl)-1,2,3,4-tetrahydroisoquinoline Dihydrochloride

To a solution of (R)-tert-butyl 7-(4-(2-(2-methylpyrrolidin-1-yl)ethyl)phenyl)-3,4-dihydroisoquinoline-2(1H)-carboxylate (0.304 g, 0.723 mmol) in methanol (10 mL) was added 2 M HCl in MeOH (0.500 mL) and the mixture was stirred for 16 h at room temperature. The solution was concentrated to provide the title compound as a white solid (260 mg). LCMS m/z=321.0 [M+H]⁺.

### Example 1.19

### Preparation of (R)-3-Methoxy-1-(7-(4-(2-(2-methylpyrrolidin-1-yl)ethyl)phenyl)-3,4-dihydroisoquinolin-2(1H)-yl)propan-1-one Hydrochloride (Compound 1)

To a solution of (R)-7-(4-(2-(2-methylpyrrolidin-1-yl)ethyl)phenyl)-1,2,3,4-tetrahydroisoquinoline (0.130 g, 0.406 mmol) in DCM (10 mL) was added triethylamine (0.226 mL, 1.62 mmol) and 3-methoxypropanoyl chloride (0.0746 g, 0.608 mmol). The reaction mixture was stirred for 1 h at room temperature. The solvent was removed under reduced pressure. The residue was dissolved in ACN/H₂O/AcOH and purified by preparative HPLC. Appropriate fractions were combined, basified with 2 M Na₂CO₃ and extracted three times with EtOAc. The combined organic phases were dried over Na₂SO₄, filtered and concentrated. The residue was dissolved in MeOH (5 mL). Then, HCl (1 M in Et₂O, 0.118 mL) was added followed by EtOAc (5 mL). The resulting mixture was concentrated to provide the hydrochloride salt of the title compound as a white solid. LCMS m/z=407.5 [M+H]⁺; ¹H NMR (400 MHz, Methanol-d₄) δ ppm 1.49 (d, J=6.32 Hz, 3H), 1.80 (s, 1H), 2.04-2.18 (m, 1H), 2.29-2.39 (m, 1H), 2.76 (q, J=6.40 Hz, 2H), 2.87 (t, J=5.81 Hz, 1H), 2.95 (t, J=5.81 Hz, 1H), 3.08-3.17 (m, 2H), 3.28 (s, 2H), 3.28-3.33 (m, 4H), 3.48-3.57 (m, 1H), 3.58-3.65 (m, 1H), 3.71 (t, J=5.81 Hz, 2H), 3.80 (t, J=5.81 Hz, 3H), 4.73-4.81 (m, 2H), 7.24 (d, J=7.58 Hz, 1H), 7.37-7.47 (m, 4H), 7.55-7.65 (m, 2H).

### Example 1.20

### Preparation of (R)-Cyclopropyl(7-(4-(2-(2-methylpyrrolidin-1-yl)ethyl)phenyl)-3,4-dihydroisoquinolin-2(1H)-yl)methanone Hydrochloride (Compound 2)

The title compound was prepared in a similar manner as stated above in Example 1.19. LCMS m/z=389.5 [M+H]⁺; ¹H NMR (400 MHz, Methanol-d₄) δ ppm 0.83-0.90 (m, 2H), 0.90-0.95 (m, 2H), 1.50 (d, J=6.06 Hz, 3H), 1.72-1.84 (m, 1H), 2.00-2.18 (m, 3H), 2.30-2.40 (m, 1H), 2.82-2.90 (m, 1H), 2.93-3.03 (m, 1H), 3.11-3.20 (m, 2H), 3.22-3.31 (m, 2H), 3.50-3.59 (m, 1H), 3.59-3.68 (m, 1H), 3.78 (s, 2H), 3.97 (s, 1H), 4.72 (s, 1H), 4.95-5.00 (m, 1H), 7.25 (s, 1H), 7.34-7.50 (m, 4H), 7.60 (s, 2H).

### Example 1.21

### Preparation of (R)-6-(4-(2-(2-Methylpyrrolidin-1-yl)ethyl)phenyl)-1,2,3,4-tetrahydroisoquinoline

### Step A: Preparation of tert-Butyl 6-Bromo-3,4-dihydroisoquinoline-2(1H)-carboxylate

To a solution of 6-bromo-1,2,3,4-tetrahydroisoquinoline hydrochloride (1.00 g, 4.02 mmol) in EtOH (20 mL) was added sodium hydrogencarbonate (1.69 g, 20.1 mmol) and di-tert-butyl dicarbonate (0.966 g, 4.43 mmol). The reaction mixture was allowed to stir for 16 h at room temperature. The reaction mixture was then concentrated. The residue was diluted with H₂O and extracted three times with EtOAc. The combined organics were dried over Na₂SO₄, filtered, and concentrated to give the title compound (1.15 g) as a clear oil. LCMS m/z=311.9 [M+H]⁺; ¹H NMR (400 MHz, Methanol-d₄) δ ppm 1.45-1.51 (m, 9H), 2.81 (t, J=5.81 Hz, 2H), 3.29-3.34 (m, 2H), 3.61 (t, J=5.68 Hz, 2H), 4.50 (s, 2H), 7.04 (d, J=8.08 Hz, 1H).

### Step B: Preparation of (R)-6-(4-(2-(2-Methylpyrrolidin-1-yl)ethyl)phenyl)-1,2,3,4-tetrahydroisoquinoline Dihydrochloride

From tert-butyl 6-bromo-3,4-dihydroisoquinoline-2(1H)-carboxylate, the dihydrochroride salt of the title compound was prepared in a similar manner (instead of HPLC, silica column chromatography was used as the purification method) as stated above in Example 1.18. LCMS m/z=321.2 [M+H]⁺.

### Example 1.22

### Preparation of (R)-3-Methoxy-1-(6-(4-(2-(2-methylpyrrolidin-1-yl)ethyl)phenyl)-3,4-dihydroisoquinolin-2(1H)-yl)propan-1-one Hydrochloride (Compound 4)

The title compound was prepared in a similar manner as stated above in Example 1.19. LCMS m/z=407.3 [M+H]⁺; ¹H NMR (400 MHz, Methanol-d₄) δ ppm 1.50 (d, J=5.56 Hz, 3H), 1.72-1.83 (m, 1H), 2.04-2.16 (m, 2H), 2.28-2.39 (m, 1H), 2.67-2.75 (m, 2H), 2.82-2.87 (m, 1H), 2.91-2.95 (m, 1H), 3.10-3.18 (m, 2H), 3.22-3.27 (m, 1H), 3.27-3.30 (m, 2H), 3.30-3.32 (m, 1H), 3.49-3.62 (m, 2H), 3.65-3.71 (m, 3H), 3.75 (d, J=4.29 Hz, 3H), 4.67 (s, 1H), 4.71 (s, 1H), 7.13-7.25 (m, 1H), 7.36-7.46 (m, 4H), 7.58 (d, J=6.32 Hz, 2H).

### Example 1.23

### Preparation of (R)-5-(4-(2-(2-Methylpyrrolidin-1-yl)ethyl)phenyl)isoindoline Dihydrochloride

The title compound was prepared in a similar manner as stated above in Example 1.21. LCMS m/z=307.4 [M+H]⁺.

### Example 1.24

### Preparation of (R)-Cyclopropyl(5-(4-(2-(2-methylpyrrolidin-1-yl)ethyl)phenyl)isoindolin-2-yl)methanone Hydrochloride (Compound 5)

The title compound was prepared in a similar manner as stated above in Example 1.19. LCMS m/z=375.3 [M+H]⁺; ¹H NMR (400 MHz, Methanol-d₄) δ ppm 0.88-0.94 (m, 2H), 0.94-0.99 (m, 2H), 1.50 (d, J=6.06 Hz, 3H), 1.77 (dd, 1H), 1.88-1.98 (m, 1H), 2.03-2.18 (m, 2H), 2.30-2.41 (m, 1H), 3.08-3.17 (m, 1H), 3.24-3.29 (m, 1H), 3.30-3.32 (m, 2H), 3.51-3.57 (m, 1H), 3.59-3.68 (m, 1H), 3.73-3.81 (m, 1H), 4.76 (d, J=10.11 Hz, 2H), 5.08 (d, J=6.06 Hz, 2H), 7.37-7.45 (m, 3H), 7.53-7.58 (m, 2H), 7.62 (d, J=7.83 Hz, 2H).

### Example 1.25

### Preparation of (R)-3-Methoxy-1-(5-(4-(2-(2-methylpyrrolidin-1-yl)ethyl)phenyl)isoindolin-2-yl)propan-1-one Hydrochloride (Compound 6)

The title compound was prepared in a similar manner as stated above in Example 1.19. LCMS m/z=393.3 [M+H]⁺; ¹H NMR (400 MHz, Methanol-d₄) δ ppm 1.18-1.27 (m, 1H), 1.51 (d, J=5.81 Hz, 3H), 1.73-1.85 (m, 1H), 1.96-2.02 (m, 1H), 2.04-2.18 (m, 2H), 2.29-2.40 (m, 1H), 2.67 (t, J=5.56 Hz, 2H), 3.09-3.17 (m, 2H), 3.21-3.32 (m, 3H), 3.50-3.66 (m, 2H), 3.72 (t, J=5.94 Hz, 2H), 3.75-3.81 (m, 1H), 4.70 (d, J=12.88 Hz, 2H), 4.88 (d, J=4.55 Hz, 2H), 7.31-7.36 (m, 1H), 7.41 (d, J=7.33 Hz, 2H), 7.48-7.54 (m, 2H), 7.59 (d, J=7.58 Hz, 2H).

### Example 1.26

### Preparation of (R)-(5-(4-(2-(2-Methylpyrrolidin-1-yl)ethyl)phenyl)isoindolin-2-yl)(tetrahydro-2H-pyran-4-yl)methanone Hydrochloride (Compound 12)

The title compound was prepared in a similar manner as stated above in Example 1.19. LCMS m/z=419.3 [M+H]⁺; ¹H NMR (400 MHz, Methanol-d₄) δ ppm 1.34 (d, J=6.57 Hz, 3H), 1.58-1.69 (m, 1H), 1.70-1.77 (m, 2H), 1.79-1.89 (m, 2H), 1.92-2.03 (m, 2H), 2.14-2.26 (m, 1H), 2.81-2.97 (m, 3H), 2.99-3.15 (m, 2H), 3.33-3.46 (m, 1H), 3.48-3.60 (m, 2H), 3.96-4.05 (m, 2H), 4.78 (d, J=9.60 Hz, 2H), 5.02 (d, J=7.83 Hz, 2H), 7.34-7.42 (m, 3H), 7.52-7.64 (m, 4H).

### Example 1.27

### Preparation of (R)-4-Methoxy-1-(5-(4-(2-(2-methylpyrrolidin-1-yl)ethyl)phenyl)isoindolin-2-yl)butan-1-one Hydrochloride (Compound 17)

The title compound was prepared in a similar manner as stated above in Example 1.19. LCMS m/z=407.3 [M+H]⁺; ¹H NMR (400 MHz, Methanol-d₄) δ ppm 1.48 (d, J=6.57 Hz, 3H), 1.71-1.82 (m, 1H), 1.89-1.99 (m, 2H), 2.04-2.18 (m, 2H), 2.30-2.40 (m, 1H), 2.54 (t, J=7.45 Hz, 2H), 3.05-3.19 (m, 2H), 3.22-3.29 (m, 2H), 3.32-3.35 (m, 3H), 3.48 (t, J=6.19 Hz, 2H), 3.50-3.57 (m, 1H), 3.58-3.69 (m, 1H), 3.71-3.81 (m, 1H), 4.77 (d, J=9.85 Hz, 2H), 7.37-7.44 (m, 3H), 7.53-7.59 (m, 2H), 7.63 (d, J=8.08 Hz, 2H).

### Example 1.28

### Preparation of (R)-2-Hydroxy-1-(6-(4-(2-(2-methylpyrrolidin-1-yl)ethyl)phenyl)-3,4-dihydroisoquinolin-2(1H)-yl)ethanone Hydrochloride (Compound 10)

### Step A: Preparation of 1-(6-Bromo-3,4-dihydroisoquinolin-2(1H)-yl)-2-hydroxyethanone

A magnetically stirred flask equipped with a drying tube was charged with a solution of 6-bromo-1,2,3,4-tetrahydroisoquinoline (3.43 g, 16.2 mmol) in anhydrous toluene (40 mL), to which was added 2,2-dimethyl-1,3-dioxolan-4-one (1.9 g, 16.2 mmol). The resulting solution was refluxed for 20 h. The reaction mixture was cooled, extracted with 1 N HCl (30 mL), followed by brine (20 mL), and the organic extract was dried over MgSO₄. The resulting solution was reduced in volume to about 25 mL, and heptane (25 mL) was gradually added over 20 min as precipitate formed. The resulting white solid was collected by filtration and rinsed with 1:1 toluene/heptane to provide the title compound. LCMS m/z=270.1 [M+H]⁺; ¹H NMR (400 MHz, CDCl₃) δ 2.48 (bs, 1H), 2.87-2.94 (m, 2H), 3.55 (t, J=5.9 Hz, 1.2H), 3.89 (t, J=6.1 Hz, 0.8H), 4.26 (s, 2H), 4.40 (s, 0.8H), 4.75 (s, 1.2H), 7.00 (d, J=8.3 Hz, 0.4H), 7.07 (d, J=8.3 Hz, 0.6H), 7.32-7.39 (m, 2H).

### Step B: Preparation of 2-Hydroxy-1-(6-(4,4,5,5-tetramethyl-1,3,2-dioxaborolan-2-yl)-3,4-dihydroisoquinolin-2(1H)-yl)ethanone

To a solution of 1-(6-bromo-3,4-dihydroisoquinolin-2(1H)-yl)-2-hydroxyethanone (2.08 g, 7.70 mmol) in anhydrous toluene (50 mL) was added potassium acetate (1.66 g, 17 mmol) and the resulting mixture was stirred well. 4,4,4′,4′,5,5,5′,5′-Octamethyl-2,2′-bi(1,3,2-dioxaborolane) (2.9 g, 11.5 mmol) was added, followed by triphenylphosphine (120 mg, 0.45 mmol) and trans-dichlorobis(triphenylphosphine)palladium (II) (160 mg, 0.23 mmol). The mixture was stirred under N₂ at 100° C. for 2 h. Upon cooling, water was added, and the aqueous layer was discarded. The organic extract was dried over MgSO₄ and reduced to 50 mL total volume, and this was in turn diluted with 50 mL heptane, resulting in the formation of precipitate over 15 min, which was collected by filtration to provide the title compound. LCMS m/z=318.4 [M+H]⁺; ¹H NMR (400 MHz, CDCl₃) δ 1.37 (s, 12H), 2.39 (bs, 1H), 2.90-2.97 (m, 2H), 3.51 (t, J=6.0 Hz, 1.2H), 3.90 (t, J=6.1 Hz, 0.8H), 4.25-4.28 (m, 2H), 4.46 (s, 0.8H), 4.82 (s, 1.2H), 7.13 (d, J=7.6 Hz, 0.4H), 7.19 (d, J=7.6 Hz, 0.6H), 7.63 (s, 1H), 7.64-7.69 (m, 1H).

### Step C: Preparation of (R)-2-Hydroxy-1-(6-(4-(2-(2-methylpyrrolidin-1-yl)ethyl)phenyl)-3,4-dihydroisoquinolin-2(1H)-yl)ethanone Hydrochloride (Compound 10)

In a 20 mL vial, a solution of (R)-1-(4-bromophenethyl)-2-methylpyrrolidine (790 mg, 2.5 mmol) in ethanol (10 mL) was treated with 2-hydroxy-1-(6-(4,4,5,5-tetramethyl-1,3,2-dioxaborolan-2-yl)-3,4-dihydroisoquinolin-2(1H)-yl)ethanone (790 mg, 2.5 mmol). The mixture was stirred at 60° C. until solids were dissolved, then a solution of NaHCO₃ (2.0 M aq, 3.0 mL, 6 mmol) was added, followed by dichloro[1,1′-bis(diphenylphosphino)ferrocene]palladium (II) dichloromethane adduct (100 mg, 0.125 mmol) and toluene (3 mL). The vial was capped, and the reaction mixture was heated at 60° C. for 2 h. The reaction mixture was diluted with water (20 mL). The resulting suspension was filtered, and the organic layer was separated. The aqueous layer was extracted with dichloromethane (3×20 mL). The organic phases were combined and purified by flash chromatography, eluting with 5% 7 N NH₃/MeOH in dichloromethane. The combined fractions were concentrated to dryness, and the residue was taken up in hot isopropyl acetate (50 mL) and filtered. The filtrate was concentrated to 10 mL. The precipitate was collected, providing a tan solid. The solid was then dissolved in hot ethanol (10 mL), to which was added 1.25 M HCl/ethanol (1.0 mL). The resulting solution was cooled to 15° C. for 20 min. The precipitate was collected by filtration to provide the title compound as a tan solid. LCMS m/z=379.5 [M+H]⁺; ¹H NMR (400 MHz, DMSO-d₆) δ 1.20 (d, J=6.6 Hz, 0.4H), 1.42 (d, J=6.6 Hz, 2.6H), 1.58-1.69 (m, 1H), 1.90-2.00 (m, 2H), 2.16-2.24 (m, 1H), 2.82-2.95 (m, 2H), 3.04-3.12 (m, 2H), 3.13-3.22 (m, 2H), 3.39-3.46 (m, 1H), 3.47-3.55 (m, 1H), 3.58-3.67 (m, 2H), 3.70-3.75 (m, 1H), 4.19 (s, 2H), 4.55-4.69 (m, 3H), 7.23-7.32 (m, 1H), 7.40 (d, J=8.2 Hz, 2H), 7.45-7.52 (m, 2H), 7.63 (d, J=8.5 Hz, 2H), 10.39 (bs, 1H).

### Example 1.29

### Preparation of 1-((R)-1-Methyl-8-(4-(2-((R)-2-methylpyrrolidin-1-yl)ethyl)phenyl)-4,5-dihydro-1H-benzo[d]azepin-3(2H)-yl)ethanone (Compound 24)

### Step A: Preparation of (R)-1-(8-Chloro-1-methyl-4,5-dihydro-1H-benzo[d]azepin-3(2H)-yl)ethanone

To a solution of (R)-8-chloro-1-methyl-2,3,4,5-tetrahydro-1H-benzo[d]azepine hydrochloride (0.108 g, 0.465 mmol) in DCM (3 mL) was added triethylamine (0.259 mL, 1.861 mmol) and acetyl chloride (0.043 mL, 0.605 mmol). The reaction was stirred for 30 min at room temperature. The mixture was concentrated to give the title compound (304 mg) without further purification. LCMS m/z=238.1 [M+H]⁺.

### Step B: Preparation of 1-((R)-1-Methyl-8-(4-(2-((R)-2-methylpyrrolidin-1-yl)ethyl)phenyl)-4,5-dihydro-1H-benzo[d]azepin-3(2H)-yl)ethanone

In a Smith Microwave Synthesizer vial were placed (R)-1-(8-chloro-1-methyl-4,5-dihydro-1H-benzo[d]azepin-3(2H)-yl)ethanone (0.100 g, 0.421 mmol), (R)-4-(2-(2-methylpyrrolidin-1-yl)ethyl)phenylboronic acid, HCl (0.136 g, 0.505 mmol), potassium acetate (0.124 g, 1.262 mmol), Pd(OAc)₂ (1.889 mg, 8.41 μmmol), X-Phos (10.03 mg, 0.021 mmol), and THF (5 mL). The reaction was heated at 120° C. under microwave irradiation for 1 h. The reaction mixture was diluted with water and the organic phase was separated. The aqueous layer was extracted with EtOAc. The combined organic phases were concentrated, dissolved in ACN/H₂O and purified by HPLC to give the TFA salt of the title compound as a white solid (0.030 mg). LCMS m/z=391.6 [M+H]⁺; ¹H NMR (400 MHz, Methanol-d₄) δ ppm 1.48 (d, J=6.57 Hz, 3H), 1.71-1.81 (m, 1H), 1.90-1.99 (m, 2H), 2.03-2.18 (m, 1H), 2.29-2.40 (m, 1H), 2.54 (t, J=7.45 Hz, 2H), 3.02-3.19 (m, 2H), 3.22-3.36 (m, 7H), 3.48 (t, J=6.19 Hz, 2H), 3.50-3.58 (m, 1H), 3.59-3.70 (m, 1H), 3.70-3.82 (m, 1H), 4.77 (d, J=9.85 Hz, 2H), 4.92-4.95 (m, 1H), 7.41 (t, J=7.83 Hz, 3H), 7.54-7.59 (m, 2H), 7.63 (d, J=8.08 Hz, 2H).

### Example 1.30

### Preparation of (R)-(5-(4-(2-(2-Methylpyrrolidin-1-yl)ethyl)phenyl)isoindolin-2-yl)(pyridin-4-yl)methanone (Compound 25)

### Step A: Preparation of 5-Bromoisoindoline

To a solution of 5-bromoisoindoline-1,3-dione (8.117 g, 35.9 mmol) in THF (10 mL) was added boron trifluoride (8.40 mL, 108 mmol) dropwise. The reaction mixture was heated to reflux. After refluxing temperature was reached, borane tetrahydrofuran complex (144 mL, 144 mmol) was added dropwise and the reaction was stirred at reflux for 16 h. The mixture was cooled to 0° C., cautiously treated with 3 N HCl (10 mL), and stirred at room temperature for 1 h. The aqueous mixture was washed with EtOAc, and the organic phase was discarded. The aqueous layer was basified (pH 9-10) and extracted with EtOAc. The organic phase was washed with brine, dried over MgSO₄, filtered, and concentrated under reduced pressure to give the title compound (4.02 g). LCMS m/z=198.0 [M+H]⁺.

### Step B: Preparation of (R)-5-(4-(2-(2-Methylpyrrolidin-1-yl)ethyl)phenyl)isoindoline

To a Smith Microwave Synthesizer vial was added 5-bromoisoindoline (4.02 g 20.3 mmol), (R)-4-(2-(2-methylpyrrolidin-1-yl)ethyl)phenylboronic acid hydrochloride (5.47 g, 20.3 mmol), aqueous Na₂CO₃ (2 M solution, 25.4 mL, 50.7 mmol), and Pd(PPh₃)₄ (0.704 g, 0.609 mmol) in a mixture of EtOH (10 mL) and benzene (30 mL). The resulting reaction mixture was heated at 100° C. under microwave irradiation for 120 min. The reaction mixture was diluted with water and extracted with EtOAc. The combined organic phases were washed with brine, dried over sodium sulfate, and concentrated under reduced pressure. The residue was purified by silica gel column chromatography to give the title compound as a brown solid (1.27 g). LCMS m/z=307.4 [M+H]⁺.

### Step C: Preparation of (R)-(5-(4-(2-(2-Methylpyrrolidin-1-yl)ethyl)phenyl)isoindolin-2-yl)(pyridin-4-yl)methanone

To a solution of (R)-5-(4-(2-(2-methylpyrrolidin-1-yl)ethyl)phenyl)isoindoline (0.200 g, 0.653 mmol) in dichloromethane (5 mL) was added triethylamine (0.091 mL, 0.653 mmol) and isonicotinoyl chloride hydrochloride (0.116 g, 0.653 mmol). The reaction was stirred at room temperature for 2 h. The combined organic phases were concentrated and purified by HPLC. The combined fractions were basified with 2 M Na₂CO₃ and extracted three times with EtOAc. The combined organic phases were dried over Na₂SO₄, filtered, and concentrated to give the hydrochloride salt of the title compound as a white solid (0.093 g). LCMS m/z=412.3 [M+H]⁺; ¹H NMR (400 MHz, Methanol-d₄) δ ppm 1.37-1.43 (m, 3H), 1.45 (none, 1H), 1.61-1.74 (m, 1H), 1.94-2.12 (m, 2H), 2.21-2.31 (m, 1H), 2.97-3.11 (m, 2H), 3.14-3.20 (m, 2H), 3.23-3.28 (m, 1H), 3.41-3.48 (m, 1H), 3.50-3.60 (m, 1H), 3.63-3.75 (m, 1H), 4.80 (s, 1H), 4.99 (d, J=9.35 Hz, 2H), 7.34 (t, J=8.21 Hz, 2H), 7.42 (d, J=4.04 Hz, 1H), 7.49-7.60 (m, 4H), 8.27 (d, J=5.81 Hz, 2H), 8.99 (d, J=5.56 Hz, 2H).

### Example 1.31

### Preparation of (R)-(5-(4-(2-(2-Methylpyrrolidin-1-yl)ethyl)phenyl)isoindolin-2-yl)(pyridin-3-yl)methanone (Compound 28)

From (R)-5-(4-(2-(2-methylpyrrolidin-1-yl)ethyl)phenyl)isoindoline and nicotinoyl chloride, using a similar method to the one described in Example 1.30, Step C, the hydrochloride salt of the title compound was obtained. LCMS m/z=412.3 [M+H]⁺; ¹H NMR (400 MHz, Methanol-d₄) δ ppm 1.39 (dd, J=6.32, 3.79 Hz, 3H), 1.60-1.73 (m, 1H), 1.93-2.09 (m, 2H), 2.19-2.31 (m, 1H), 2.96-3.08 (m, 2H), 3.12-3.20 (m, 2H), 3.39-3.47 (m, 1H), 3.50-3.59 (m, 1H), 3.61-3.71 (m, 1H), 4.89 (d, J=8.34 Hz, 2H), 4.98 (d, J=9.35 Hz, 2H), 7.33 (t, J=7.71 Hz, 2H), 7.37-7.44 (m, 1H), 7.46-7.59 (m, 4H), 8.13 (dd, J=7.71, 5.94 Hz, 1H), 8.82 (d, J=7.83 Hz, 1H), 8.92 (d, J=5.56 Hz, 1H), 9.13 (s, 1H).

### Example 1.32

### Preparation of (R)-(5-(4-(2-(2-Methylpyrrolidin-1-yl)ethyl)phenyl)isoindolin-2-yl)(pyrimidin-5-yl)methanone (Compound 29)

In a 10 mL microwave vial were placed (R)-5-(4-(2-(2-methylpyrrolidin-1-yl)ethyl)phenyl)isoindoline (0.100 g, 0.326 mmol), pyrimidine-5-carboxylic acid (0.0607 g, 0.489 mmol), triethylamine (0.0910 mL, 0.653 mmol), PS-carbodiimide (1.02 g, 1.63 mmol), and CH₂Cl₂ (3 mL). The reaction was heated under microwave irradiation at 120° C. for 1 h. The mixture was cooled to room temperature and filtered. The filtrate was concentrated under reduced pressure and the residue was purified by HPLC to give the TFA salt of the title compound as a white solid (0.060 mg). LCMS m/z=413.2 [M+H]⁺; ¹H NMR (400 MHz, Methanol-d₄) δ ppm 1.26-1.31 (m, 3H), 1.48 (dd, J=6.44, 3.66 Hz, 4H), 2.10 (s, 2H), 3.03-3.18 (m, 2H), 3.44-3.58 (m, 1H), 3.69-3.80 (m, 1H), 4.91-4.96 (m, 1H), 4.96-5.00 (m, 1H), 5.01-5.08 (m, 2H), 7.34-7.50 (m, 4H), 7.54-7.67 (m, 4H), 9.10 (s, 2H), 9.30 (s, 1H).

### Example 1.33

### Preparation of (R)-(5-(4-(2-(2-Methylpyrrolidin-1-yl)ethyl)phenyl)isoindolin-2-yl)(pyridin-2-yl)methanone (Compound 30)

From (R)-5-(4-(2-(2-methylpyrrolidin-1-yl)ethyl)phenyl)isoindoline and picolinic acid, using a similar method to the one described in Example 1.32, the TFA salt of the title compound was obtained. LCMS m/z=412.3 [M+H]⁺; ¹H NMR (400 MHz, Methanol-d₄) δ ppm 1.27-1.33 (m, 1H), 1.48 (dd, J=6.57, 3.03 Hz, 3H), 1.71-1.80 (m, 1H), 2.04-2.19 (m, 2H), 2.31-2.40 (m, 1H), 3.04-3.16 (m, 2H), 3.22-3.28 (m, 1H), 3.49-3.57 (m, 1H), 3.60-3.68 (m, 1H), 3.71-3.80 (m, 1H), 5.04 (d, J=8.34 Hz, 2H), 5.17 (d, J=8.34 Hz, 2H), 7.32-7.49 (m, 3H), 7.55 (d, J=8.08 Hz, 2H), 7.63 (dd, J=10.48, 8.21 Hz, 3H), 7.88 (d, J=7.83 Hz, 1H), 7.96-8.03 (m, 1H), 8.65-8.71 (m, 1H).

### Example 1.34

### Preparation of (R)-2-Methoxy-1-(5-(4-(2-(2-methylpyrrolidin-1-yl)ethyl)phenyl)isoindolin-2-yl)ethanone (Compound 33)

From (R)-5-(4-(2-(2-methylpyrrolidin-1-yl)ethyl)phenyl)isoindoline and 2-methoxyacetyl chloride, using a similar method to the one described in Example 1.30, Step C, the hydrochloride salt of the title compound was obtained. LCMS m/z=379.3 [M+H]⁺; ¹H NMR (400 MHz, Methanol-d₄) δ ppm 1.50 (d, J=6.06 Hz, 3H), 1.73-1.84 (m, 1H), 2.06-2.22 (m, 2H), 2.30-2.43 (m, 1H), 3.05-3.20 (m, 2H), 3.25-3.29 (m, 1H), 3.47-3.50 (m, 4H), 3.53-3.60 (m, 2H), 3.62-3.74 (m, 1H), 3.76-3.83 (m, 1H), 4.25 (d, J=2.27 Hz, 2H), 4.84 (d, J=11.37 Hz, 3H), 7.44 (d, J=8.08 Hz, 3H), 7.55-7.59 (m, 1H), 7.61-7.67 (m, 3H).

### Example 1.35

### Preparation of (R)-2-Hydroxy-1-(5-(4-(2-(2-methylpyrrolidin-1-yl)ethyl)phenyl)isoindolin-2-yl)ethanone (Compound 34)

From (R)-5-(4-(2-(2-methylpyrrolidin-1-yl)ethyl)phenyl)isoindoline and 2-hydroxyacetic acid, using a similar method to the one described in Example 1.32, the hydrochloride salt of the title compound was obtained. LCMS m/z=365.5 [M+H]⁺; ¹H NMR (400 MHz, Methanol-d₄) δ ppm 1.13-1.27 (m, 2H), 1.39 (d, J=6.32 Hz, 3H), 1.58-1.75 (m, 1H), 1.93-2.12 (m, 2H), 2.17-2.31 (m, 1H), 2.94-3.10 (m, 2H), 3.10-3.19 (m, 1H), 3.37-3.50 (m, 1H), 3.47-3.61 (m, 1H), 3.62-3.75 (m, 1H), 4.19 (d, J=5.05 Hz, 2H), 4.63-4.74 (m, 4H), 7.23-7.36 (m, 3H), 7.40-7.55 (m, 4H).

### Example 1.36

### Preparation of 1-((R)-9-Fluoro-1-methyl-8-(4-(2-((R)-2-methylpyrrolidin-1-yl)ethyl)phenyl)-4,5-dihydro-1H-benzo[d]azepin-3(2H)-yl)ethanone (Compound 35)

From (R)-4-(2-(2-methylpyrrolidin-1-yl)ethyl)phenylboronic acid and (R)-1-(8-chloro-9-fluoro-1-methyl-4,5-dihydro-1H-benzo[d]azepin-3(2H)-yl)ethanone, using a similar method to the one described in Example 1.29, Step B, the TFA salt of the title compound was obtained. LCMS m/z=409.5 [M+H]⁺; ¹H NMR (400 MHz, Methanol-d₄) δ ppm 1.24 (d, J=6.95 Hz, 1H), 1.29 (d, J=7.07 Hz, 2H), 1.47 (d, J=6.57 Hz, 3H), 1.70-1.83 (m, 1H), 2.02-2.09 (m, 2H), 2.11-2.19 (m, 2H), 2.29-2.41 (m, 1H), 2.87-3.03 (m, 1H), 3.04-3.18 (m, 2H), 3.19-3.29 (m, 2H), 3.33-3.41 (m, 1H), 3.45-3.55 (m, 2H), 3.58-3.70 (m, 2H), 3.72-3.79 (m, 2H), 3.79-3.87 (m, 1H), 3.88-3.97 (m, 1H), 3.96-4.08 (m, 1H), 7.34-7.38 (m, 1H), 7.41 (d, J=7.96 Hz, 2H), 7.54 (d, J=1.52 Hz, 1H), 7.58-7.64 (m, 2H).

### Example 1.37

### Preparation of 1-((S)-9-Chloro-1-methyl-7-(4-(2-((R)-2-methylpyrrolidin-1-yl)ethyl)phenyl)-4,5-dihydro-1H-benzo[d]azepin-3(2H)-yl)ethanone (Compound 36)

From (R)-4-(2-(2-methylpyrrolidin-1-yl)ethyl)phenylboronic acid and (5)-1-(8,9-dichloro-1-methyl-4,5-dihydro-1H-benzo[d]azepin-3(2H)-yl)ethanone, using a similar method to the one described in Example 1.29, Step B, the TFA salt of the title compound was obtained. LCMS m/z=426.4 [M+H]⁺; ¹H NMR (400 MHz, Methanol-d₄) δ ppm 1.24 (d, J=6.95 Hz, 1H), 1.29 (d, J=7.07 Hz, 2H), 1.47 (d, J=6.57 Hz, 3H), 1.70-1.83 (m, 1H), 2.02-2.11 (m, 2H), 2.12-2.18 (m, 2H), 2.25-2.41 (m, 1H), 2.87-3.02 (m, 1H), 3.09 (d, J=5.94 Hz, 1H), 3.12-3.20 (m, 1H), 3.21-3.29 (m, 2H), 3.34-3.41 (m, 1H), 3.43-3.58 (m, 2H), 3.58-3.70 (m, 2H), 3.71-3.79 (m, 2H), 3.81-3.87 (m, 1H), 3.89-3.96 (m, 1H), 3.98-4.09 (m, 1H), 7.34-7.38 (m, 1H), 7.41 (d, J=7.96 Hz, 2H), 7.54 (d, J=1.52 Hz, 1H), 7.56-7.64 (m, 2H).

### Example 138

### Preparation of 1-(7-Hydroxy-1-methyl-8-(4-(2-((R)-2-methylpyrrolidin-1-yl)ethyl)phenyl)-4,5-dihydro-1H-benzo[d]azepin-3(2H)-yl)ethanone (Compound 39)

From (R)-4-(2-(2-methylpyrrolidin-1-yl)ethyl)phenylboronic acid and 1-(8-chloro-7-hydroxy-1-methyl-4,5-dihydro-1H-benzo[d]azepin-3(2H)-yl)ethanone, using a similar method to the one described in Example 1.29, Step B, the TFA salt of the title compound was obtained. LCMS m/z=407.5 [M+H]⁺; ¹H NMR (400 MHz, Methanol-d₄) δ ppm 1.23-1.34 (m, 5H), 1.47 (d, J=6.57 Hz, 6H), 2.10-2.16 (m, 4H), 2.99-3.16 (m, 4H), 3.17-3.29 (m, 2H), 3.44-3.58 (m, 3H), 3.59-3.68 (m, 2H), 3.70-3.79 (m, 1H), 3.80-3.92 (m, 1H), 6.67 (d, J=6.57 Hz, 1H), 7.04 (d, J=7.58 Hz, 1H), 7.33 (d, J=8.08 Hz, 2H), 7.55 (d, J=8.08 Hz, 2H).

### Example 1.39

### Preparation of 1-(7-Methoxy-1-methyl-8-(4-(2-((R)-2-methylpyrrolidin-1-yl)ethyl)phenyl)-4,5-dihydro-1H-benzo[d]azepin-3(2H)-yl)ethanone (Compound 41)

From (R)-4-(2-(2-methylpyrrolidin-1-yl)ethyl)phenylboronic acid and 1-(8-chloro-7-methoxy-1-methyl-4,5-dihydro-1H-benzo[d]azepin-3(2H)-yl)ethanone, using a similar method to the one described in Example 1.29, Step B, the TFA salt of the title compound was obtained. LCMS m/z=421.4 [M+H]⁺; ¹H NMR (400 MHz, Methanol-d₄) δ ppm 1.24-1.35 (m, 4H), 1.47 (d, J=6.57 Hz, 3H), 2.00-2.10 (m, 1H), 2.10-2.15 (m, 3H), 2.30-2.40 (m, 1H), 2.84-3.00 (m, 1H), 3.01-3.18 (m, 3H), 3.20-3.29 (m, 4H), 3.47-3.58 (m, 3H), 3.58-3.72 (m, 2H), 3.76 (d, J=2.53 Hz, 4H), 3.82-3.88 (m, 1H), 6.85 (d, J=5.56 Hz, 1H), 7.07 (d, J=7.96 Hz, 1H), 7.33 (d, J=7.96 Hz, 2H), 7.47 (d, J=8.08 Hz, 2H).

### Example 1.40

### Preparation of 3-Methoxy-1-((R)-1-methyl-8-(4-(2-((R)-2-methylpyrrolidin-1-yl)ethyl)phenyl)-4,5-dihydro-1H-benzo[d]azepin-3(2H)-yl)propan-1-one (Compound 26)

### Step A: Preparation of (R)-1-(8-Chloro-1-methyl-1,2,4,5-tetrahydrobenzo[d]azepin-3-yl)-3-methoxypropan-1-one

To a solution of (R)-8-chloro-1-methyl-2,3,4,5-tetrahydro-1H-benzo[d]azepine hydrochloride (0.100 g, 0.431 mmol) in DCM was added 3-methoxypropanoyl chloride (0.158 g, 1.29 mmol) followed by pyridine (0.158 mL, 1.94 mmol). The reaction was stirred for 4 h at room temperature. The mixture was diluted with EtOAc (15 mL) and washed with water (25 mL). The organic phase was dried over MgSO₄, filtered, and concentrated to give the title compound (0.121 g). LCMS m/z=435.5 [M+H]⁺; ¹H NMR (400 MHz, Methanol-d₄) δ ppm 1.29-1.37 (m, 3H), 1.47 (d, J=6.57 Hz, 3H), 1.69-1.80 (m, 1H), 2.02-2.17 (m, 2H), 2.29-2.39 (m, 1H), 2.65-2.70 (m, 2H), 2.84-2.98 (m, 1H), 3.03-3.19 (m, 3H), 3.21-3.29 (m, 6H), 3.46-3.68 (m, 6H), 3.72-3.80 (m, 2H), 3.83-3.97 (m, 1H), 7.19 (t, J=7.33 Hz, 1H), 7.39 (q, J=7.33 Hz, 4H), 7.60 (d, J=8.08 Hz, 2H).

### Example 1.41

### Preparation of Cyclopropyl((R)-1-methyl-8-(4-(2-((R)-2-methylpyrrolidin-1-yl)ethyl)phenyl)-4,5-dihydro-1H-benzo[d]azepin-3(2H)-yl)methanone (Compound 27)

From (R)-8-chloro-1-methyl-2,3,4,5-tetrahydro-1H-benzo[d]azepine hydrochloride and cyclopropanecarbonyl chloride, using a similar method to the one described in Example 1.40, the TFA salt of the title compound was obtained. LCMS m/z=417.3 [M+H]⁺; ¹H NMR (400 MHz, Methanol-d₄) δ ppm 0.66-0.91 (m, 4H), 1.28-1.39 (m, 3H), 1.47 (d, J=6.57 Hz, 3H), 1.69-1.80 (m, 1H), 1.95-2.19 (m, 3H), 2.30-2.39 (m, 1H), 2.83-2.90 (m, 1H), 2.97-3.20 (m, 3H), 3.21-3.29 (m, 3H), 3.34-3.79 (m, 5H), 3.84-4.16 (m, 2H), 7.20 (dd, J=14.02, 7.71 Hz, 1H), 7.35-7.43 (m, 4H), 7.60 (d, J=8.34 Hz, 2H).

### Example 1.42

### Preparation of 2-Hydroxy-1-(6-(4-(2-(2-methylpyrrolidin-1-yl)ethyl)phenyl)-3,4-dihydroisoquinolin-2(1H)-yl)ethanone (Compound 50)

### Step A: Preparation of 4-Bromophenethyl-(2-methyl)-pyrrolidine

To a solution of 4-bromophenethyl methanesulfonate (1.0 g, 3.58 mmol) in acetonitrile (12 mL) was added potassium carbonate (1.040 g, 7.52 mmol) and stirred well. 2-Methylpyrrolidine (0.439 mL, 4.30 mmol) was added and the mixture was heated to 60° C. under N₂. Deionized water (600 μL) was added and the reaction was stirred overnight at 60° C. Additional 2-methylpyrrolidine (100 μL) was added and the reaction was stirred at 60° C. for 1 h. Additional 2-methylpyrrolidine (100 μL) was added and the reaction was stirred at 60° C. for 2 h. After cooled to room temperature, the reaction mixture was filtered. The filtrate was concentrated. The residue was diluted with water and extracted with EtOAc (twice). The EtOAc layer was extracted with 2 N HCl (twice). The aqueous phases were combined, cooled by an ice bath, slowly basified to pH ˜12 by addition of 50% aqueous NaOH solution, and then extracted with EtOAc (twice). The combined organic extracts were washed with water, dried over Na₂SO₄ and concentrated under reduced pressure to give the title compound as an oil (844 mg). LCMS m/z=268.1 [M+H]⁺.

### Step B: Preparation of 2-Hydroxy-1-(6-(4-(2-(2-methylpyrrolidin-1-yl)ethyl)phenyl)-3,4-dihydroisoquinolin-2(1H)-yl)ethanone

To a solution of 1-(4-bromophenethyl)-2-methylpyrrolidine (0.25 g, 0.932 mmol) in MeOH (2.0 mL) was added 2-hydroxy-1-(6-(4,4,5,5-tetramethyl-1,3,2-dioxaborolan-2-yl)-3,4-dihydroisoquinolin-2(1H)-yl)ethanone (0.325 g, 1.025 mmol) and stirred to obtain a slurry. The mixture was heated to 50° C. to obtain a clear solution. A solution of Na₂CO₃ (0.198 g, 1.864 mmol) in water (2.0 mL) was added (Note: Na₂CO₃ solution was prepared by warming at ˜60° C.; and the warm solution was added to the reaction mixture). 10% Pd—C (Degussa) (50 mg) was added at 55° C. and the reaction mixture was heated at 80° C. (oil bath temperature) for 2 h. Additional 10% Pd—C (20 mg) was added and heated for 1 h. Additional 2-hydroxy-1-(6-(4,4,5,5-tetramethyl-1,3,2-dioxaborolan-2-yl)-3,4-dihydroisoquinolin-2(1H)-yl)ethanone (50 mg) was added and heated for 2 h (total 5 h). The reaction was cooled to room temperature and left stirring overnight. The reaction mixture was filtered through a plug of celite. The organic solvent was removed and the aqueous residue was diluted with water and extracted with EtOAc. The EtOAc layer was extracted with 2 N HCl (twice). The acid layer was cooled by an ice bath and slowly basified to pH 12 by addition of 50% aqueous NaOH solution and extracted with EtOAc (twice). The combined organic extracts were washed with water (twice), dried over Na₂SO₄ and concentrated under reduced pressure. The residue was purified by HPLC to give the TFA salt of the title compound (115.4 mg). LCMS m/z=379.4 [M+H]⁺.

### Example 1.43

### Preparation of (R)-(6-Hydroxypyridin-2-yl)(6-(4-(2-(2-methylpyrrolidin-1-yl)ethyl)phenyl)-3,4-dihydroisoquinolin-2(1H)-yl)methanone (Compound 31)

To a solution of 6-hydroxypicolinic acid (0.021 g, 0.150 mmol) and DCC resin (0.635 g, 0.749 mmol) in DCM was added (R)-6-(4-(2-(2-methylpyrrolidin-1-yl)ethyl)phenyl)-1,2,3,4-tetrahydroisoquinoline (0.040 g, 0.125 mmol) and triethylamine (0.035 mL, 0.250 mmol). The reaction was stirred overnight at room temperature. Additional DCC resin (3.17 g, 3.75 mmol) and 6-hydroxypicolinic acid (0.0294 g, 0.210 mmol) was added. The reaction was stirred overnight at 40° C. The mixture was filtered and concentrated. The residue was purified by HPLC and converted to HCl salt of the title compound (4.2 mg). LCMS m/z=442.6 [M+H]⁺.

### Example 1.44

### Preparation of (R)-(6-Methoxypyridin-3-yl)(6-(4-(2-(2-methylpyrrolidin-1-yl)ethyl)phenyl)-3,4-dihydroisoquinolin-2(1H)-yl)methanone (Compound 32)

To a solution of 6-methoxynicotinic acid (0.023 g, 0.150 mmol) and DCC resin (0.635 g, 0.749 mmol) in DCM was added (R)-6-(4-(2-(2-methylpyrrolidin-1-yl)ethyl)phenyl)-1,2,3,4-tetrahydroisoquinoline (0.040 g, 0.125 mmol) and triethylamine (0.035 mL, 0.250 mmol). The reaction was stirred overnight at room temperature. The mixture was filtered and concentrated. The residue was purified by HPLC and converted to HCl salt of the title compound (4.4 mg). LCMS m/z=456.4 [M+H]⁺.

### Example 1.45

### Preparation of 2-Hydroxy-1-(6-(4-(2-(piperidin-1-yl)ethyl)phenyl)-3,4-dihydroisoquinolin-2(1H)-yl)ethanone (Compound 43)

### Step A: Preparation of 2-(6-Bromo-3,4-dihydroisoquinolin-2(1H)-yl)-2-oxoethyl Acetate

To a solution of 6-bromo-1,2,3,4-tetrahydroisoquinoline hydrochloride (0.400 g, 1.609 mmol) and triethylamine (1.122 mL, 8.05 mmol) in DCM (10 mL) was added 2-chloro-2-oxoethyl acetate (0.242 g, 1.770 mmol). The reaction was stirred at room temperature for 30 min. The mixture was diluted with DCM, washed with 1 M HCl, brine, dried over Na₂SO₄, and concentrated to give the title compound without further purification.

### Step B: Preparation of 2-Hydroxy-1-(6-(4-(2-hydroxyethyl)phenyl)-3,4-dihydroisoquinolin-2(1H)-yl)ethanone

To a microwave vial were added 2-(6-bromo-3,4-dihydroisoquinolin-2(1H)-yl)-2-oxoethyl acetate (0.529 g, 1.695 mmol), 4-(2-(tert-butyldimethylsilyloxy)ethyl)phenylboronic acid (0.475 g, 1.695 mmol), NaHCO₃ (1.695 mL, 3.39 mmol), and Pd(PPh₃)₄ (0.059 g, 0.051 mmol) dissolved in a 1:3 mixture of ethanol:benzene. The vial was sealed under argon, and placed on the microwave for 3 h at 100° C. The reaction mixture was filtered, and diluted with ethyl acetate. The organic layer was washed with 10% aqueous NaOH, brine, dried over Na₂SO₄ and concentrated to give the title compound. LCMS m/z=312.2 [M+H]⁺.

### Step C: Preparation of 2-(4-(1,2,3,4-Tetrahydroisoquinolin-6-yl)phenyl)ethanol

To a solution of 2-hydroxy-1-(6-(4-(2-hydroxyethyl)phenyl)-3,4-dihydroisoquinolin-2(1H)-yl)ethanone (0.450 g, 1.445 mmol) in MeOH (10 mL) was added NaOH (0.289 g, 7.23 mmol). The reaction was stirred for 20 min to give the title compound. LCMS m/z=254.4 [M+H]⁺.

### Step D: Preparation of 6-(4-(2-(tert-butyldimethylsilyloxy)ethyl)phenyl)-1,2,3,4-tetrahydroisoquinoline

To a solution of 2-(4-(1,2,3,4-tetrahydroisoquinolin-6-yl)phenyl)ethanol (0.193 g, 0.762 mmol) and triethylamine (0.117 mL, 0.838 mmol) in DCM (10 mL) was added tert-butylchlorodimethylsilane (0.126 g, 0.838 mmol) and stirred overnight at room temperature. Additional tert-butylchlorodimethylsilane (0.115 g, 0.762 mmol) was added and the reaction was stirred at room temperature for 8 h. The mixture was diluted with ethyl acetate, washed with water, brine, dried over Na₂SO₄, and concentrated to give the title compound without further purification.

### Step E: Preparation of 2-(6-(4-(2-(tert-butyldimethylsilyloxy)ethyl)phenyl)-3,4-dihydroisoquinolin-2(1H)-yl)-2-oxoethyl Acetate

To a solution of 6-(4-(2-(tert-butyldimethylsilyloxy)ethyl)phenyl)-1,2,3,4-tetrahydroisoquinoline (0.200 g, 0.544 mmol) and triethylamine (0.379 mL, 2.72 mmol) in DCM (10 mL) was added 2-chloro-2-oxoethyl acetate (0.082 g, 0.598 mmol). The reaction mixture was stirred at room temperature for 30 min. Upon completion, the reaction mixture was diluted with DCM, washed with 1 M HCl, brine, dried over Na₂SO₄, and concentrated to give the title compound. LCMS m/z=468.6 [M+H]⁺.

### Step F: Preparation of 2-(6-(4-(2-Hydroxyethyl)phenyl)-3,4-dihydroisoquinolin-2(1H)-yl)-2-oxoethyl Acetate

To a solution of 2-(6-(4-(2-(tert-butyldimethylsilyloxy)ethyl)phenyl)-3,4-dihydroisoquinolin-2(1H)-yl)-2-oxoethyl acetate (0.180 g, 0.385 mmol) in THF (8 mL) was added HBr (0.025 mL, 0.462 mmol). The reaction was stirred at room temperature for 1.5 h. Upon completion of the reaction, saturated NaHCO₃ solution was added and the mixture was extracted with ethyl acetate. The organic layer was washed with brine, dried over Na₂SO₄, and concentrated to give the title compound as a white solid. LCMS m/z=354.2 [M+H]⁺.

### Step G: Preparation of 2-oxo-2-(6-(4-(2-(tosyloxy)ethyl)phenyl)-3,4-dihydroisoquinolin-2(1H)-yl)ethyl Acetate

To a solution of 2-(6-(4-(2-hydroxyethyl)phenyl)-3,4-dihydroisoquinolin-2(1H)-yl)-2-oxoethyl acetate (0.054 g, 0.153 mmol) and triethylamine (0.085 mL, 0.611 mmol) in DCM (1 mL) was added 4-methylbenzene-1-sulfonyl chloride (0.029 g, 0.153 mmol). The reaction was stirred for 30 min. Upon completion, the mixture was extracted with DCM. The organic layer was washed with water, dried over Na₂SO₄, and concentrated to give the title compound.

### Step H: Preparation of 2-oxo-2-(6-(4-(2-(piperidin-1-yl)ethyl)phenyl)-3,4-dihydroisoquinolin-2(1H)-yl)ethyl Acetate

In a microwave vial was placed 2-oxo-2-(6-(4-(2-(tosyloxy)ethyl)phenyl)-3,4-dihydroisoquinolin-2(1H)-yl)ethyl acetate (0.035 g, 0.069 mmol), piperidine (6.46 mg, 0.076 mmol), and Na₂CO₃ (0.029 g, 0.276 mmol) in acetonitrile (3 mL). The reaction was heated under microwave irradiation at 100° C. for 3 h. Upon completion, the reaction mixture was filtered and concentrated to give the title compound.

### Step I: Preparation of 2-Hydroxy-1-(6-(4-(2-(piperidin-1-yl)ethyl)phenyl)-3,4-dihydroisoquinolin-2(1H)-yl)ethanone

To a solution of 2-oxo-2-(6-(4-(2-(piperidin-1-yl)ethyl)phenyl)-3,4-dihydroisoquinolin-2(1H)-yl)ethyl acetate (0.029 g, 0.069 mmol) in 2-propanol (1 mL) was added HCl (4.0 M in dioxane, 0.017 mL, 0.069 mmol). The reaction was stirred for 1 h at room temperature. After being neutralized it was filtered through a silica pad. The filtrate was concentrated and the residue was purified by HPLC to give the TFA salt of the title compound (6.6 mg). LCMS m/z=379.3 [M+H]⁺. ¹H NMR (400 MHz, Methanol-d₄) δ ppm 1.47-1.64 (m, 1H), 1.72-1.92 (m, 3H), 1.99 (d, J=14.65 Hz, 2H), 2.89-3.05 (m, 4H), 3.06-3.16 (m, 2H), 3.32-3.39 (m, 2H), 3.58-3.70 (m, 3H), 3.83 (t, J=5.68 Hz, 1H), 4.33 (s, 2H), 4.61 (s, 1H), 4.74 (s, 1H), 7.24 (d, J=7.83 Hz, 1H), 7.36 (d, J=8.08 Hz, 2H), 7.40-7.48 (m, 2H), 7.60 (d, J=8.08 Hz, 2H).

### Example 1.46

### Preparation of 2-hydroxy-1-(6-(4-(2-morpholinoethyl)phenyl)-3,4-dihydroisoquinolin-2(1H)-yl)ethanone (Compound 45)

Using morpholine, the title compound was prepared in a similar manner to the one described in Example 1.45. LCMS m/z=381.1 [M+H]⁺. ¹H NMR (400 MHz, Methanol-d₄) δ ppm 2.89-3.01 (m, 2H), 3.06-3.16 (m, 2H), 3.17-3.27 (m, 2H), 3.38-3.48 (m, 2H), 3.51-3.70 (m, 3H), 3.70-3.90 (m, 3H), 3.99-4.17 (m, 2H), 4.33 (s, 2H), 4.61 (s, 1H), 4.74 (s, 1H), 7.24 (d, J=8.08 Hz, 1H), 7.37 (d, J=8.08 Hz, 2H), 7.40-7.48 (m, 2H), 7.61 (d, J=8.08 Hz, 2H).

### Example 1.47

### Preparation of (R)-1-(5-Chloro-6-(4-(2-(2-methylpyrrolidin-1-yl)ethyl)phenyl)-3,4-dihydroisoquinolin-2(1H)-yl)ethanone (Compound 37)

### Step A: Preparation of 2-Chloro-1-methoxy-3-(2-nitrovinyl)benzene

2-Chloro-3-methoxybenzaldehyde (2.614 g, 15.32 mmol) and ammonium acetate (1.181 g, 15.32 mmol) was dissolved in acetic acid (12.26 mL). The reaction was added nitromethane (4.13 mL, 77 mmol) and warmed to 40° C. overnight and 85° C. for 6 h. The solvent was removed under reduced pressure and the residue was purified by column chromatography to give the title compound (3.073 g). ¹H NMR (400 MHz, CDCl₃) δ ppm 3.88-3.98 (m, 3H), 7.05 (dd, J=8.27, 1.33 Hz, 1H), 7.18 (dd, J=7.89, 1.33 Hz, 1H), 7.24-7.34 (m, 1H), 7.57 (d, J=13.64 Hz, 1H), 8.45 (d, J=13.64 Hz, 1H).

### Step B: Preparation of 2-(2-Chloro-3-methoxyphenyl)ethanamine

To a solution of 2-chloro-1-methoxy-3-(2-nitrovinyl)benzene (2.878 g, 13.47 mmol) in tetrahydrofuran (53.9 mL) cooled to −20° C. was added lithium aluminum hydride (1 M in tetrahydrofuran) (53.9 mL, 53.9 mmol). The reaction was warmed to 50° C. After 2 h, water and ethyl acetate were added. The mixture was filtered and the filter cake was rinsed with ethyl acetate. The organic layer of the filtrate was separated, dried over sodium sulfate and concentrated under reduced pressure. The residue was purified by silica gel column chromatography to give the title compound (1.188 g). LCMS m/z=186.0 [M+H]⁺; ¹H NMR (400 MHz, Methanol-d₄) δ ppm 3.07-3.21 (m, 4H), 3.82-3.92 (m, 3H), 6.95 (dd, J=7.64, 1.20 Hz, 1H), 7.02 (dd, J=8.34, 1.14 Hz, 1H), 7.21-7.31 (m, 1H).

### Step C: Preparation of 5-Chloro-6-methoxy-1,2,3,4-tetrahydroisoquinoline

To a solution of 2-(2-chloro-3-methoxyphenyl)ethanamine, HCl (0.426 g, 1.918 mmol) in water (1.534 mL) was added formalin (37%) (0.200 mL, 2.69 mmol). The reaction was stirred at 85° C. for 2 h and then diluted with water and dichloromethane. The organic layer was separated. The aqueous layer was extracted with dichloromethane. The combined organic layers were dried over sodium sulfate and concentrated under reduced pressure. The residue was suspended in dichloroethane (15.34 mL), trifluoroacetic acid (12.70 mL, 165 mmol) was added and the mixture was stirred at 80° C. for 3 h. The solvent was removed under reduced pressure. The residue was purified by HPLC to give the TFA salt of the title compound (500 mg). LCMS m/z=198.2 [M+H]⁺; ¹H NMR (400 MHz, Methanol-d₄) δ ppm 3.13 (t, J=6.57 Hz, 2H), 3.55 (t, J=6.51 Hz, 2H), 3.88-3.93 (m, 3H), 4.33 (s, 2H), 7.07 (d, J=8.59 Hz, 1H), 7.19 (d, J=8.59 Hz, 1H).

### Step D: Preparation of 1-(5-Chloro-6-hydroxy-3,4-dihydroisoquinolin-2(1H)-yl)ethanone

To a solution of 5-chloro-6-methoxy-1,2,3,4-tetrahydroisoquinoline (TFA salt) (0.105 g, 0.337 mmol) in tetrahydrofuran (2.81 mL) was added triethylamine (0.188 mL, 1.348 mmol) followed by acetyl chloride (0.031 mL, 0.438 mmol). The reaction was stirred for 15 min and then diluted with ethyl acetate (50 mL). The ethyl acetate solution was washed with 1 M HCl (2×10 mL), then brine (10 mL), dried over sodium sulfate and concentrated. The residue was then dissolved in dichloromethane (3.38 mL). Boron tribromide (1 M in dichloromethane) (0.845 mL, 0.845 mmol) was added and the reaction was stirred for 1 h at room temperature. The mixture was diluted with ethyl acetate (50 mL), washed with 1 M HCl (2×10 mL), then brine (10 mL), dried over sodium sulfate and concentrated under reduced pressure. The residue was purified by HPLC to give the TFA salt of the title compound (57 mg). LCMS m/z=226.3 [M+H]⁺; ¹H NMR (400 MHz, CDCl₃) δ ppm 2.15-2.21 (m, 3H), 2.81-2.99 (m, 2H), 3.65-3.90 (m, 2H), 4.61 (d, J=49.52 Hz, 2H), 5.54-6.00 (m, 1H), 6.87-7.00 (m, 2H).

### Step E: Preparation of (R)-1-(5-Chloro-6-(4-(2-(2-methylpyrrolidin-1-yl)ethyl)phenyl)-3,4-dihydroisoquinolin-2(1H)-yl)ethanone

To a solution of 1-(5-chloro-6-hydroxy-3,4-dihydroisoquinolin-2(1H)-yl)ethanone in dichloromethane was added pyridine (0.012 mL, 0.146 mmol) followed by trifluoromethanesulfonic anhydride (0.012 mL, 0.073 mmol). The reaction was stirred for 1 h and diluted with ethyl acetate. The ethyl acetate solution was washed with 1 M HCl (twice), brine, dried over sodium sulfate and concentrated under reduced pressure. The residue was placed in a microwave vial with (R)-4-(2-(2-methylpyrrolidin-1-yl)ethyl)phenylboronic acid (0.011 g, 0.048 mmol) and tetrakis(triphenylphosphine)palladium(0) (1.647 mg, 1.426 μmol). Benzene (3 mL) and ethanol (1 mL) was added followed by sodium carbonate (0.048 mL, 0.095 mmol). The mixture was heated under microwave irradiation for 1 h at 120° C. The organic layer was separated, filtered and concentrated under reduced pressure. The residue was purified by HPLC to give the TFA salt of the title compound (6.1 mg). LCMS m/z=397.2 [M+H]⁺; ¹H NMR (400 MHz, Methanol-d₄) δ ppm 1.33 (d, J=6.95 Hz, 0.3H), 1.47 (d, J=6.57 Hz, 2.7H), 1.70-1.81 (m, 1H), 2.02-2.18 (m, 2H), 2.20 (d, J=5.05 Hz, 3H), 2.30-2.41 (m, 1H), 2.93 (t, J=6.06 Hz, 1H), 3.03 (t, J=6.19 Hz, 1H), 3.05-3.20 (m, 2H), 3.23-3.34 (m, 2H), 3.50-3.57 (m, 1H), 3.60-3.69 (m, 1H), 3.72-3.79 (m, 1H), 3.79-3.87 (m, 2H), 4.75 (d, J=5.43 Hz, 2H), 7.16-7.22 (m, 2H), 7.35-7.40 (m, 4H).

### Example 1.48

### Preparation of (R)-1-(5-fluoro-6-(4-(2-(2-methylpyrrolidin-1-yl)ethyl)phenyl)-3,4-dihydroisoquinolin-2(1H)-yl)ethanone (Compound 38)

From 2-fluoro-3-methoxybenzaldehyde, using a similar method to the one described in Example 1.47, the TFA salt of the title compound was obtained. LCMS m/z=381.2 [M+H]⁺; ¹H NMR (400 MHz, Methanol-d₄) δ ppm 1.32 (d, J=6.82 Hz, 0.3H), 1.46 (d, J=7.07 Hz, 2.7H), 1.69-1.82 (m, 1H), 1.99-2.14 (m, 2H), 2.14-2.22 (m, 2H), 2.28-2.41 (m, 1H), 2.82-2.88 (m, 1H), 2.95 (t, J=5.94 Hz, 1H), 3.02-3.18 (m, 2H), 3.21-3.33 (m, 2H), 3.47-3.57 (m, 1H), 3.58-3.69 (m, 1H), 3.70-3.87 (m, 3H), 4.74 (d, J=5.56 Hz, 2H), 7.07 (t, J=9.03 Hz, 1H), 7.25-7.36 (m, 2H), 7.40 (d, J=8.08 Hz, 2H), 7.52 (dd, J=8.08, 1.39 Hz, 2H).

### Example 1.49

### Preparation of (R)-1-(7-methyl-6-(4-(2-(2-methylpyrrolidin-1-yl)ethyl)phenyl)-3,4-dihydroisoquinolin-2(1H)-yl)ethanone (Compound 40)

### Step A: Preparation of 2-(3-Methoxy-4-methylphenyl)ethanamine

A solution of 2-(3-methoxy-4-methylphenyl)acetonitrile (2.008 g, 12.46 mmol) and concentrated HCl (1.544 mL, 18.68 mmol) in ethanol (49.8 mL) was flushed with argon and palladium on carbon (0.795 g, 7.47 mmol) was added. The reaction was stirred overnight under H₂. Additional palladium on carbon was added and the reaction was stirred overnight under H₂. The mixture was filtered and the filtrate was concentrated to give the hydrochloride salt of the title compound as a white solid (2.41 g). LCMS m/z=314.3 [M+H]⁺.

### Step B: Preparation of (R)-1-(7-Methyl-6-(4-(2-(2-methylpyrrolidin-1-yl)ethyl)phenyl)-3,4-dihydroisoquinolin-2(1H)-yl)ethanone

From 2-(3-methoxy-4-methylphenyl)ethanamine, using a similar method to the one described in Example 1.47, Step C to E, the title compound was obtained. LCMS m/z=377.4 [M+H]⁺; ¹H NMR (400 MHz, Methanol-d₄) δ ppm 1.32 (d, J=6.82 Hz, 0.3H), 1.45-1.49 (d, J=6.82 Hz, 2.7H), 1.70-1.81 (m, 1H), 2.02-2.16 (m, 2H), 2.16-2.20 (m, 6H), 2.30-2.40 (m, 1H), 2.81 (t, J=5.94 Hz, 1H), 2.90 (t, J=5.81 Hz, 1H), 3.01-3.20 (m, 2H), 3.22-3.32 (m, 2H), 3.49-3.58 (m, 1H), 3.60-3.68 (m, 1H), 3.71-3.81 (m, 3H), 4.68 (d, J=7.33 Hz, 2H), 6.97 (s, 1H), 7.07 (d, J=12.00 Hz, 1H), 7.28 (d, J=8.08, 2H), 7.35-7.39 (m, 2H).

### Example 1.50

### Preparation of (R)-1-(7-(4-(2-(2-Methylpyrrolidin-1-yl)ethyl)phenyl)-4,5-dihydro-1H-benzo[d]azepin-3(2H)-yl)ethanone (Compound 42)

In a microwave vial was placed 3-acetyl-2,3,4,5-tetrahydro-1H-benzo[d]azepin-7-yl trifluoromethanesulfonate (0.028 g, 0.083 mmol), (R)-4-(2-(2-methylpyrrolidin-1-yl)ethyl)phenylboronic acid hydrochloride (0.022 g, 0.083 mmol), tetrakis(triphenylphosphine)palladium(0) (2.88 mg, 2.490 μmol), 2 M Na₂CO₃ (0.083 mL, 0.166 mmol), benzene (0.215 mL) and EtOH (0.072 mL). The reaction was heated under microwave irradiation for 1 h at 120° C. The organic layer was separated and concentrated. The residue was purified by HPLC to give the title compound as a viscous, white solid (10 mg). LCMS m/z=377.4 [M+H]⁺.

### Example 1.51

### Preparation of (R)-1-(7-Methoxy-6-(4-(2-(2-methylpyrrolidin-1-yl)ethyl)phenyl)-3,4-dihydroisoquinolin-2(1H)-yl)ethanone (Compound 44)

### Step A: Preparation of Methyl 3-bromo-4-methoxyphenethylcarbamate

To a solution of 2-(3-bromophenyl)ethanamine (4.812 g, 24.05 mmol) in tetrahydrofuran (96 mL) cooled in an ice-bath were add triethylamine (6.70 mL, 48.1 mmol) and methyl chloroformate (2.79 mL, 36.1 mmol). The reaction was slowly warmed to room temperature and stirred for 1 h. The reaction mixture was dilute with ethyl acetate, washed with 1 M HCl (twice) and brine, dried over sodium sulfate and concentrated under reduced pressure to give the title compound as a white solid (6.2 g). LCMS m/z=288.1 [M+H]⁺; ¹H NMR (400 MHz, CDCl₃) δ ppm 2.73 (t, J=6.95 Hz, 2H), 3.36-3.41 (m, 2H), 3.66 (s, 3H), 3.88 (s, 3H), 4.67 (s, 1H), 6.84 (d, J=8.34 Hz, 1H), 7.09 (dd, J=8.34, 2.02 Hz, 1H), 7.37 (d, J=2.15 Hz, 1H). Step B: Preparation of 6-Bromo-7-methoxy-3,4-dihydroisoquinolin-1(2H)-one

A mixture of polyphosphoric acid (3.98 mL, 7.11 mmol) and methyl 3-bromo-4-methoxyphenethylcarbamate (2.05 g, 7.11 mmol) was heated to 120° C. for 5.5 h. The reaction mixture was extracted twice with ethyl acetate, and the organic layer was washed with water and brine. The combined organic phases were dried over sodium sulfate and concentrated under reduced pressure to give the title compound (637 mg). LCMS m/z=256.1 [M+H]⁺.

### Step C: Preparation of (R)-7-Methoxy-6-(4-(2-(2-methylpyrrolidin-1-yl)ethyl)phenyl)-3,4-dihydroisoquinolin-1(2H)-one

In a microwave vial were placed 6-bromo-7-methoxy-3,4-dihydroisoquinolin-1(2H)-one (0.627 g, 2.45 mmol), (R)-4-(2-(2-methylpyrrolidin-1-yl)ethyl)phenylboronic acid, HCl (0.660 g, 2.45 mmol), tetrakis(triphenylphosphine)palladium(0) (0.085 g, 0.073 mmol) benzene (9 mL), EtOH (3 mL) and 2 M sodium carbonate (2.448 mL, 4.90 mmol). The reaction was heated under microwave irradiation for 1 h at 120° C. The organic layer was separated and concentrated under reduced pressure. The residue was purified by preparative HPLC. The HPLC fractions containing the product were combined and freed of organic solvent under reduced pressure. The aqueous phase was made basic with 2 M Na₂CO₃, saturated with sodium chloride and extracted with ethyl acetate three times. The organic extracts were dried over magnesium sulfate and filtered. HCl (1 M in ether, 5 mL) was added to the filtrate and the mixture concentrated under reduced pressure. The residue was resuspended in water, frozen and lyophilized to give the HCl salt of the title compound (0.305 g). LCMS m/z=365.5 [M+H]⁺; ¹H NMR (400 MHz, Methanol-d₄) δ ppm 1.35 (d, J=6.57 Hz, 0.3H), 1.51 (d, J=6.44 Hz, 2.7H), 1.74-1.85 (m, 1H), 2.05-2.22 (m, 2H), 2.38 (d, J=7.58 Hz, 1H), 2.97 (t, J=6.69 Hz, 2H), 3.08-3.21 (m, 2H), 3.26-3.34 (m, 2H), 3.51-3.61 (m, 3H), 3.61-3.72 (m, 1H), 3.75-3.83 (m, 1H), 3.85 (s, 3H), 7.24 (s, 1H), 7.40 (d, J=7.96 Hz, 2H), 7.54 (d, J=7.96 Hz, 2H), 7.63 (s, 1H).

### Step D: Preparation of (R)-1-(7-Methoxy-6-(4-(2-(2-methylpyrrolidin-1-yl)ethyl)phenyl)-3,4-dihydroisoquinolin-2(1H)-yl)ethanone

To a solution of lithium aluminum hydride (1 M in tetrahydrofuran) (0.423 mL, 0.423 mmol) cooled to −20° C. was added (R)-7-methoxy-6-(4-(2-(2-methylpyrrolidin-1-yl)ethyl)phenyl)-3,4-dihydroisoquinolin-1(2H)-one (HCl salt) (0.077 g, 0.211 mmol) in tetrahydrofuran (3.02 mL). The reaction was refluxed for 3 h, worked up with 20% NaOH solution and added ethyl acetate. The mixture was filtered and the filter cake was rinsed with ethyl acetate. The ethyl acetate layer was separated, washed with brine and dried over sodium sulfate. To the solution was added HCl (1 M in diethyl ether, 0.5 mL) and the mixture was concentrated. The residue was dissolved in chloroform (3.25 mL), triethylamine (0.068 mL, 0.488 mmol) and acetyl chloride (0.015 mL, 0.211 mmol) were added, and the mixture was stirred at room temperature for 30 min. The solvent was removed under reduced pressure. The residue was purified by HPLC to give the TFA salt of the title compound (36 mg). LCMS m/z=393.4 [M+H]⁺; ¹H NMR (400 MHz, Methanol-d₄) δ ppm 1.32 (d, J=6.82 Hz, 0.3H), 1.46 (d, J=6.32 Hz, 2.7H), 1.68-1.80 (m, 1H), 1.99-2.16 (m, 2H), 2.17-2.21 (m, 3H), 2.28-2.40 (m, 1H), 2.79 (t, J=5.81 Hz, 1H), 2.88 (t, J=5.68 Hz, 1H), 2.98-3.17 (m, 2H), 3.19-3.30 (m, 2H), 3.48-3.56 (m, 1H), 3.58-3.68 (m, 1H), 3.71-3.79 (m, 6H), 4.67-4.73 (m, 2H), 6.87 (d, J=11.62 Hz, 1H), 7.06 (s, 1H), 7.32 (d, J=8.08 Hz, 2H), 7.46 (d, J=8.08 Hz, 2H).

### Example 1.52

### Preparation of (R)-1-(7-Hydroxy-6-(4-(2-(2-methylpyrrolidin-1-yl)ethyl)phenyl)-3,4-dihydroisoquinolin-2(1H)-yl)ethanone (Compound 46)

To a solution of (R)-1-(7-methoxy-6-(4-(2-(2-methylpyrrolidin-1-yl)ethyl)phenyl)-3,4-dihydroisoquinolin-2(1H)-yl)ethanone and TFA (0.027 g, 0.053 mmol) in dichloromethane (0.533 mL) was added boron tribromide (1 M in dichloromethane) (0.133 mL, 0.133 mmol). The reaction was stirred at room temperature for 1 h. The mixture was concentrated under reduced pressure. The residue was purified by HPLC to give the TFA salt of the title compound (16 mg). LCMS m/z=379.5 [M+H]⁺; ¹H NMR (400 MHz, Methanol-d₄) δ ppm 1.32 (d, J=7.07 Hz, 0.3H), 1.46 (d, J=6.57 Hz, 2.7H), 1.68-1.80 (m, 1H), 1.99-2.15 (m, 2H), 2.15-2.18 (m, 3H), 2.28-2.39 (m, 1H), 2.77 (t, J=5.94 Hz, 1H), 2.85 (t, J=5.81 Hz, 1H), 2.97-3.16 (m, 2H), 3.20-3.30 (m, 2H), 3.47-3.56 (m, 1H), 3.58-3.67 (m, 1H), 3.68-3.79 (m, 3H), 4.59-4.65 (m, 2H), 6.64-6.72 (m, 1H), 7.04 (s, 1H), 7.32 (d, J=8.08 Hz, 2H), 7.54 (d, J=8.34 Hz, 2H).

### Example 1.53

### Preparation of 1-(1-Methyl-6-(4-(2-((R)-2-methylpyrrolidin-1-yl)ethyl)phenyl)-3,4-dihydroisoquinolin-2(1H)-yl)ethanone (Compound 47)

### Step A: Preparation of 6-Methoxy-1-methyl-3,4-dihydroisoquinoline

To a solution of 2-(3-methoxyphenyl)ethanamine (10.058 g, 66.5 mmol) and triethylamine (27.8 mL, 200 mmol) in dichloromethane (266 mL) cooled in an ice-bath was added acetyl chloride (7.09 mL, 100 mmol) dropwise. The reaction was stirred at room temperature for 90 min and diluted with dichloromethane. The organic solution was washed with 1 M HCl and brine, dried over sodium sulfate and concentrated under reduced pressure. The residue was dissolved in toluene (84 mL) and warmed to 40° C. Phosphorus oxychloride (11.10 mL, 119 mmol) was added dropwise. The reaction was refluxed for 2 h. After cooling, the reaction was quenched with H₂O and extracted several times with dichloromethane to get rid of organic impurities. The aqueous layer was basified with 50% sodium hydroxide solution to pH 10, then extracted several times with dichloromethane. The combined organic layers were washed with brine, dried over magnesium sulfate and concentrated under reduced pressure to give the title compound as an orange oil (9.68 g). LCMS m/z=177.3 [M+H]⁺; ¹H NMR (400 MHz, Methanol-d₄) δ ppm 2.32-2.35 (m, 3H), 2.70-2.76 (m, 2H), 3.52-3.60 (m, 2H), 3.86 (d, 3H), 6.81 (d, J=2.53 Hz, 1H), 6.88 (dd, J=8.59, 2.53 Hz, 1H), 7.56 (d, J=8.59 Hz, 1H).

### Step B: Preparation of 6-Methoxy-1-methyl-1,2,3,4-tetrahydroisoquinoline

To a solution of 6-methoxy-1-methyl-3,4-dihydroisoquinoline (2.71 g, 15.47 mmol) in methanol (61.9 mL) was added sodium borohydride (1.170 g, 30.9 mmol) in portions. The reaction was stirred at room temperature for 1 h, quenched with water and freed of the organic solvent under reduced pressure. The aqueous residue was extracted with ethyl acetate, washed with brine, dried over sodium sulfate and concentrated under reduced pressure to give the title compound (12.56 g). LCMS m/z=178.4 [M+H]⁺; NMR (400 MHz, Methanol-d₄) δ ppm 1.43 (d, J=6.57 Hz, 3H), 2.69-2.76 (m, 1H), 2.83-2.97 (m, 2H), 3.17-3.24 (m, 1H), 3.76 (s, 3H), 3.99 (q, J=6.65 Hz, 1H), 6.64 (d, J=2.53 Hz, 1H), 6.73 (dd, J=8.59, 2.53 Hz, 1H), 7.08 (d, J=8.59 Hz, 1H).

### Step C: Preparation of 1-(1-Methyl-6-(4-(2-((R)-2-methylpyrrolidin-1-yl)ethyl)phenyl)-3,4-dihydroisoquinolin-2(1H)-yl)ethanone

From 6-methoxy-1-methyl-1,2,3,4-tetrahydroisoquinoline, using a similar method to the one described in Example 1.47, Steps D and E, the TFA salt of the title compound was obtained. LCMS m/z=377.5 [M+H]⁺; ¹H NMR (400 MHz, Methanol-d₄) δ ppm 1.32 (d, J=6.82 Hz, 0.3H), 1.46 (t, J=6.19 Hz, 3.7H), 1.57 (d, J=6.82 Hz, 1H), 1.69-1.81 (m, 1H), 2.00-2.17 (m, 2H), 2.18 (s, 2H), 2.22 (s, 1H), 2.29-2.40 (m, 1H), 2.84-2.93 (m, 1H), 2.93-3.03 (m, 1H), 3.02-3.19 (m, 3H), 3.21-3.29 (m, 2H), 3.48-3.67 (m, 3H), 3.70-3.79 (m, 1H), 3.90-3.98 (m, 0.66H), 4.51-4.58 (m, 0.34H), 5.11-5.18 (m, 0.34H), 5.54-5.61 (m, 0.66H), 7.21-7.29 (m, 1H), 7.35-7.41 (m, 3H), 7.43-7.47 (m, 1H), 7.60 (d, J=8.34 Hz, 2H).

### Example 1.54

### Preparation of (R)-2-Hydroxy-1-(7-(4-(2-(2-methylpyrrolidin-1-yl)ethyl)phenyl)-4,5-dihydro-1H-benzo[d]azepin-3(2H)-yl)ethanone (Compound 48)

### Step A: Preparation of (R)-7-(4-(2-(2-Methylpyrrolidin-1-yl)ethyl)phenyl)-2,3,4,5-tetrahydro-1H-benzo[d]azepine

To a solution of (R)-1-(7-(4-(2-(2-methylpyrrolidin-1-yl)ethyl)phenyl)-4,5-dihydro-1H-benzo[d]azepin-3(2H)-yl(ethanone (TFA salt) (0.092 g, 0.244 mmol) in methanol (9.05 mL) was added 50% aqueous solution of sodium hydroxide (3 mL, 57 mmol). The reaction was stirred at 70° C. overnight. Additional sodium hydroxide solution (3 mL), H₂O and methanol were added and the reaction was stirred at 90° C. overnight. The mixture was freed of the organic solvent. The aqueous layer was saturated with sodium chloride and extracted with ethyl acetate ten times. To the combined organic extracts was added HCl (1 M in diethyl ether, 2 mL) and the mixture was concentrated under reduced pressure. The residue was resuspended in water, frozen and lyophilized to give the hydrochloride salt of the title compound (68 mg). LCMS m/z=335.6 [M+H]⁺.

### Step B: Preparation of (R)-2-Hydroxy-1-(7-(4-(2-(2-methylpyrrolidin-1-yl)ethyl)phenyl)-4,5-dihydro-1H-benzo[d]azepin-3(2H)-yl)ethanone

To a solution of (R)-7-(4-(2-(2-methylpyrrolidin-1-yl)ethyl)phenyl)-2,3,4,5-tetrahydro-1H-benzo[d]azepine dihydrochloride salt (0.022 g, 0.054 mmol) in acetonitrile (1.080 mL) and water (0.270 mL) was added triethylamine (0.023 mL, 0.162 mmol) followed by acetoxyacetyl chloride (8.71 μL, 0.081 mmol). The reaction was stirred for 30 min. 50% Aqueous sodium hydroxide solution (0.255 mL, 4.86 mmol) and MeOH (0.5 mL) were added and the reaction was stirred for another 30 min. The mixture was concentrated under reduced pressure. The residue was purified by HPLC to give the TFA salt of the title compound (18 mg). LCMS m/z=393.4 [M+H]⁺; ¹H NMR (400 MHz, Methanol-d₄) δ ppm 1.32 (d, J=6.82 Hz, 0.3H), 1.46 (d, J=6.57 Hz, 2.7H), 1.69-1.81 (m, 1H), 2.00-2.19 (m, 2H), 2.29-2.40 (m, 1H), 2.93-3.04 (m, 4H), 3.05-3.17 (m, 2H), 3.21-3.30 (m, 2H), 3.48-3.56 (m, 3H), 3.58-3.67 (m, 1H), 3.70-3.79 (m, 3H), 4.29 (s, 2H), 7.21 (d, J=7.58 Hz, 1H), 7.34-7.43 (m, 4H), 7.60 (d, J=8.08 Hz, 2H).

### Example 1.55

### Preparation of (R)-1-(7-Fluoro-6-(4-(2-(2-methylpyrrolidin-1-yl)ethyl)phenyl)-3,4-dihydroisoquinolin-2(1H)-yl)ethanone (Compound 49)

From 4-fluoro-3-methoxybenzaldehyde, using a similar method to the one described in Example 1.47, the TFA salt of the title compound was obtained. LCMS m/z=381.4 [M+H]⁺; ¹H NMR (400 MHz, Methanol-d₄) δ ppm 1.23 (d, J=6.82 Hz, 0.3H), 1.38 (d, J=6.57 Hz, 2.7H), 1.60-1.72 (m, 1H), 1.91-2.09 (m, 2H), 2.08-2.11 (m, 3H), 2.21-2.31 (m, 1H), 2.76 (t, J=6.06 Hz, 1H), 2.85 (t, J=5.94 Hz, 1H), 2.92-3.11 (m, 2H), 3.13-3.21 (m, 2H), 3.40-3.48 (m, 1H), 3.50-3.59 (m, 1H), 3.63-3.72 (m, 3H), 4.62 (d, J=8.84 Hz, 2H), 6.94 (t, J=11.24 Hz, 1H), 7.18 (d, J=7.83 Hz, 1H), 7.31 (d, J=8.08 Hz, 2H), 7.43 (d, J=7.33 Hz, 2H).

### Example 1.56

### Preparation of 1-(4-Methyl-6-(4-(2-((R)-2-methylpyrrolidin-1-yl)ethyl)phenyl)-3,4-dihydroisoquinolin-2(1H)-yl)ethanone (Compound 51)

### Step A: Preparation of 1-Methoxy-3-(1-nitropropan-2-yl)benzene

To a solution of methyllithium lithium bromide complex (1.5 M in ether) (14.51 mL, 21.77 mmol) cooled in an ice-bath was added copper(I) iodide (4.11 g, 21.60 mmol) followed by 1-methoxy-3-(2-nitrovinyl)benzene (3 g, 16.74 mmol) in tetrahydrofuran (59.8 mL) dropwise at 0° C. The reaction was stirred for 2 h, poured into NH₄OH (saturated with NH₄Cl) (250 mL), and extracted with diethyl ether (2×100 mL). The combined organic extracts were dried over magnesium sulfate and concentrated. The residue was purified by column chromatography to give the title compound (1.246 g). ¹H NMR (400 MHz, DMSO-d₆) δ ppm 1.25 (d, J=7.07 Hz, 3H), 3.44-3.55 (m, 1H), 3.71-3.76 (m, 3H), 4.80 (d, J=8.08 Hz, 2H), 6.81 (dd, J=8.21, 1.89 Hz, 1H), 6.85-6.92 (m, 2H), 7.23 (t, J=7.83 Hz, 1H).

### Step B: Preparation of 2-(3-Methoxyphenyl)propan-1-amine

To a solution of 1-methoxy-3-(1-nitropropan-2-yl)benzene (1.142 g, 5.85 mmol) in methanol (23.40 mL) was added palladium on carbon (10%) (0.436 g, 4.09 mmol) followed by ammonium formate (1.660 g, 26.3 mmol). The reaction was stirred at room temperature for 4 h, filtered and concentrated under reduced pressure. The residue was suspended in water with 0.5 mL of 50% sodium hydroxide and chloroform. The phases were separated and the aqueous layer was saturated with sodium chloride and extracted twice with chloroform. The combined organic layers were dried over sodium sulfate and filtered. The filtrate was treated with HCl (1 M in diethyl ether, 10 mL). The solvent removed under reduced pressure to give the title compound as a white solid (1.182 g). LCMS m/z=166.1 [M+H]⁺; ¹H NMR (400 MHz, Methanol-d₄) δ ppm 1.30-1.36 (m, 3H), 3.00-3.07 (m, 1H), 3.11-3.16 (m, 2H), 3.77-3.81 (m, 3H), 6.82-6.90 (m, 3H), 7.24-7.32 (m, 1H).

### Step C: Preparation of 1-(4-Methyl-6-(4-(2-((R)-2-methylpyrrolidin-1-yl)ethyl)phenyl)-3,4-dihydroisoquinolin-2(1H)-yl)ethanone

From 2-(3-methoxyphenyl)propan-1-amine, using a similar method to the one described in Example 1.47, Steps C, D and E, the TFA salt of the title compound was obtained. LCMS m/z=377.4 [M+H]⁺; ¹H NMR (400 MHz, Methanol-d₄) δ ppm 1.28 (d, J=7.07 Hz, 1H), 1.34 (d, J=6.82 Hz, 2H), 1.47 (d, J=6.57 Hz, 3H), 1.68-1.81 (m, 1H), 2.00-2.18 (m, 2H), 2.20 (d, J=9.35 Hz, 3H), 2.29-2.39 (m, 1H), 3.01-3.18 (m, 3H), 3.20-3.30 (m, 2H), 3.48-3.79 (m, 5H), 3.88 (dd, J=12.88, 5.31 Hz, 0.5H), 4.48 (d, J=17.43 Hz, 0.5H), 4.67 (d, J=17.43 Hz, 0.5H), 4.95 (d, J=17.43 Hz, 0.5H), 7.23 (t, J=8.46 Hz, 1H), 7.39 (d, J=8.08 Hz, 2H), 7.42-7.47 (m, 2H), 7.60 (d, J=8.34 Hz, 2H).

### Example 1.57

### Preparation of (R)-2-Hydroxy-1-(6-(4-(2-(2-methylpyrrolidin-1-yl)ethyl)phenyl)-3,4-dihydroisoquinolin-2(1H)-yl)ethanone Hydrochloride (Compound 10)

### Step A: Preparation of 1-(6-Bromo-3,4-dihydroisoquinolin-2(1H)-yl)-2-hydroxyethanone

A 4 L jacketed reactor equipped with mechanical stirrer, thermocouple, gas inlet, heating/cooling and condenser was charged with 6-bromo-1,2,3,4-tetrahydroisoquinoline hydrochloride (150 g, 603 mmol), followed by dichloromethane (2.8 L), and the resulting slurry was mechanically stirred. Glycolic acid (55.07 g, 724 mmol) was added, followed by 1-hydroxybenzotriazole hydrate (101.66 g, 672 mmol) and N-(dimethylaminopropyl), N′-ethylcarbodiimide hydrochloride (173.5 g, 905 mmol), followed by additional dichloromethane (0.2 L). With efficient stirring, 4-methylmorpholine (134.29 g, 1.327 mol) was slowly added in portions, while cooling to maintain a temperature at or below 25° C., and then the reaction mixture was stirred overnight at ambient temperature. The reaction mixture was then treated with 1 N HCl (2 L), resulting in formation of solids, which were removed by filtration. The aqueous layer was then removed, and the organic layer was washed with water (150 mL). The organic extract was dried over MgSO₄ filtered, and the solvent was removed from the filtrate to provide a golden yellow oil (190.6 g). The oil was then suspended in 1 N NaOH (1 L), and stirred until fine colorless solids separated out. The solids were collected by filtration and rinsed with water (2×200 mL). A second batch of the same scale was prepared under identical conditions, and the solids were consolidated at this stage. After filtering and rinsing with additional water (4×500 mL), the combined solid was dried in a vacuum oven at 45° C. for 48 h to provide the title compound (292 g). LCMS m/z=270.1 [M+H]⁺; ¹H NMR (400 MHz, CDCl₃) δ 2.48 (bs, 1H), 2.91 (m, 2H), 3.55 (t, J=5.9 Hz, 1.2H), 3.89 (t, J=6.1 Hz, 0.8H), 4.26 (m, 2H), 4.40 (s, 0.8H), 4.75 (s, 1.2H), 7.00 (d, J=8.3 Hz, 0.4H), 7.07 (d, J=8.3 Hz, 0.6H), 7.37 (m, 2H).

### Step B: Preparation of (R)-1-(4-Bromophenethyl)-2-methylpyrrolidine

A 4 L jacketed reactor equipped with mechanical stirrer, thermocouple, gas inlet, heating/cooling and condenser was charged with 4-bromophenethyl methanesulfonate (199.8 g, 716 mmol), followed by acetonitrile (2.2 L), and the resulting slurry was stirred efficiently. Water (270 mL) was then added, followed by gradual addition of potassium carbonate (297.2 g, 2.147 mol). (R)-2-methylpyrrolidine L-tartrate (168.8 g, 717 mmol) was then added, and the reaction mixture was heated at 71° C. overnight. The reaction mixture was cooled and the solvent was removed. The residue was suspended in water (500 mL) and extracted with isopropyl acetate (2×400 mL). The organic extracts were combined, rinsed with water (150 mL), dried over sodium sulfate, filtered and concentrated to dryness to provide a golden yellow oil (191 g). This material was combined with 185 g of material which was prepared at identical scale by the same method and dissolved in isopropyl acetate (2×500 mL). The mixture was extracted with 1 N HCl (2×300 mL and 200 mL). The acidic aqueous layer was separated and pH adjusted to 11-12 with 25% NaOH. This was then extracted with isopropyl acetate (2×350 mL, washed with water (150 mL) and dried over MgSO₄ (100 g). Upon filtration and solvent removal, a pale yellow oil was obtained to provide the title compound (337.5 g). LCMS m/z (%)=268.1 [M+H]⁺; ¹H NMR (CDCl₃, 400 MHz) δ ppm 1.16 (d, J=6.2 Hz, 3H), 1.46-1.55 (m, 1H), 1.71-1.81 (m, 1H), 1.82-1.90 (m, 1H), 1.94-2.01 (m, 1H), 2.24-2.31 (m, 1H), 2.32-2.39 (m, 1H), 2.41-2.47 (m, 1H), 2.84 (t, J=8.2 Hz, 2H), 3.01-3.08 (m, 1H), 3.26-3.31 (m, 1H), 7.11 (d, J=8.5 Hz, 2H), 7.42 (d, J=8.1 Hz, 2H).

### Step C: Preparation of (R)-4-(2-(2-Methylpyrrolidin-1-yl)ethyl)phenylboronic Acid

A 1 L 3-neck flask equipped with mechanical stirrer, thermometer, and addition funnel under N₂ was charged with a solution of (R)-1-(4-bromophenethyl)-2-methylpyrrolidine (26.8 g, 100 mmol) in anhydrous THF (250 mL). The reaction mixture was then cooled to an internal temperature of −78° C. A solution of butyllithium (2.5 M in hexane, 52 mL, 130 mmol) was added dropwise, maintaining an internal temperature<−70° C. Once addition was complete, stirring was continued an additional 15 min prior to the addition of triisopropyl borate (75 g, 400 mmol), followed by a rinse with 50 mL anhydrous THF, maintaining an internal temperature<−65° C. during addition. The reaction mixture was then allowed to warm to ambient temperature over 1.5 h, and was then quenched by dropwise addition of 2 N HCl (100 mL). The resulting mixture was stirred overnight, and the solvent volume was reduced to about 150 mL. The resulting suspension was cooled in an ice bath and filtered, rinsing sparingly with cold isopropanol. The filtrate volume was again reduced to 50 mL and the process was repeated. The filter cakes were combined, taken up in boiling isopropanol (250 mL), dissolving most, but not all of the solids. The mixture was then cooled in an ice bath and filtered, then the filtrate was concentrated to half volume and the process was repeated to provide two additional crops. A white solid was obtained as the title compound (23 g). LCMS m/z=234.3 [M+H]⁺; ¹H NMR (400 MHz, DMSO-d₆) δ ppm 1.41 (d, J=6.6 Hz, 3H), 1.59-1.68 (m, 1H), 1.89-2.00 (m, 2H), 2.15-2.22 (m, 1H), 3.00-3.07 (m, 2H), 3.11-3.19 (m, 2H), 3.37-3.50 (m, 2H), 3.57-3.65 (m, 1H), 4.80-6.75 (bs, 3H), 7.27 (d, J=7.6 Hz, 2H), 7.76 (d, J=8.2 Hz, 2H).

### Step D: Preparation of (R)-2-Hydroxy-1-(6-(4-(2-(2-methylpyrrolidin-1-yl)ethyl)phenyl)-3,4-dihydroisoquinolin-2(1H)-yl)ethanone Hydrochloride

A solution of 1-(6-bromo-3,4-dihydroisoquinolin-2(1H)-yl)-2-hydroxyethanone (20.85 g, 77.0 mmol), (R)-4-(2-(2-methylpyrrolidin-1-yl)ethyl)phenylboronic acid hydrochloride (29 g, 90% pure, 96 mmol), tetrakis(triphenylphosphine)palladium(0) (2.7 g, 2.3 mmol), and 2 N NaHCO₃ (125 mL, 250 mmol) in ethanol (150 mL) and toluene (450 mL) was heated at reflux for 3 h. The reaction mixture was then cooled, extracted with water, followed by brine. The aqueous extract was further extracted with dichloromethane, and the organic extracts were combined and then dried over MgSO₄, rinsing the drying agent cake thoroughly with dichloromethane. The solvent was removed, and the residue was taken up in toluene (150 mL), to which was added heptane (150 mL). A white solid precipitated and was collected by filtration (19.1 g). The filtrate was concentrated, and the residue (12 g) was subjected to flash chromatography, eluting 2 g of enriched product, from which an additional 900 mg of the title compound was obtained by recrystallization from toluene:heptane 1:1, to give a total of 20.0 g of the free base of the title compound. This was combined with an additional 6.5 g of material from different batches for a total of 26.5 g (70.0 mmol). This was taken up in 300 mL hot ethanol, to which was added 1.25 M HCl/ethanol (70 mL). As the solution cooled, a white solid formed. Upon gradual cooling to room temperature and further cooling in an ice bath to 10° C., the solid was collected by filtration, rinsing with a small amount of cold ethanol to provide the title compound as a white solid (24.95 g). LCMS m/z=379.5 [M+H]⁺. ¹H NMR (400 MHz, DMSO-d₆) δ ppm 1.42 (d, J=6.6 Hz, 3H), 1.64 (m, 1H), 1.96 (m, 2H), 2.20 (m, 1H), 2.86 (m, 0.8H), 2.93 (m, 1.2H), 3.08 (m, 2H), 3.18 (m, 2H), 3.43 (m, 1H), 3.50 (m, 1H), 3.60 (m, 2H), 3.72 (m, 1H), 4.19 (s, 2H), 4.62 (m, 3H), 7.27 (m, 1H), 7.40 (d, J=8.2 Hz, 2H), 7.48 (m, 2H), 7.63 (d, J=8.5 Hz, 2H), 10.39 (bs, 1H).

### Example 1.58

### Preparation of (R)-1-(6-(3-Fluoro-4-(2-(2-methylpyrrolidin-1-yl)ethyl)phenyl)-3,4-dihydroisoquinolin-2(1H)-yl)ethanone (Compound 52)

### Step A: Preparation of 2-(2-Fluoro-4-methoxyphenyl)ethanol

To a solution of 2-(2-fluoro-4-methoxyphenyl)acetic acid (5.0 g, 27 mmol) in THF (11 mL) cooled to 0° C. was added borane-THF complex (27 mL of 1.0 M solution in THF, 27 mmol). The cold bath was allowed to expire naturally while stirring overnight. After 16 h the reaction was quenched by the careful addition of water until effervescence ceased. The solution was neutralized by stirring with a saturated solution of sodium carbonate. The mixture was then extracted with MTBE (three times). The combined organic phases were washed with brine, dried over sodium sulfate, and concentrated to give the title compound as a colorless oil (4.3 g). LCMS m/z=153.2 [M−H₂O+H]⁺.

### Step B: Preparation of 2-Fluoro-4-methoxyphenethyl methanesulfonate

To a solution of 2-(2-fluoro-4-methoxyphenyl)ethanol (1.096 g, 6.44 mmol) in DCM (6.44 mL) was added triethylamine (1.346 mL, 9.66 mmol). The mixture was cooled in an ice-bath and added methanesulfonyl chloride (0.602 mL, 7.73 mmol). The ice-bath was removed and the reaction was stirred at room temperature for 1 h. The reaction was diluted with EtOAc, washed with 1 M HCl (15 mL) and brine, dried over Na₂SO₄ and concentrated to give the title compound (1.632 g).

### Step C: Preparation of (R)-1-(2-Fluoro-4-methoxyphenethyl)-2-methylpyrrolidine

To a solution of 2-fluoro-4-methoxyphenethyl methanesulfonate (1.623 g, 6.54 mmol) in acetonitrile (16.34 ml) were added (R)-2-methylpyrrolidine benzene sulfonate (1.901 g, 7.84 mmol) and potassium carbonate (2.71 g, 19.61 mmol). The reaction was heated at 60° C. overnight. The white heterogeneous mixture was cooled to room temperature and filtered. The filter cake was washed with acetonitrile. The filtrate was concentrated. The residue was dissolved in EtOAc and water together with 1 M HCl (6.5 mL). The aqueous layer was separated and the organic layer was extracted with water containing 1 mL of 1 M HCl. The combined aqueous phases were treated with 0.5 mL of 50% NaOH to adjust to pH 9, and then extracted with EtOAc. 2 M Na₂CO₃ (1 mL) and salt were added and the aqueous layer was further extracted with EtOAc (twice). The combined EtOAc extracts were dried over Na₂SO₄ and concentrated to give the title compound as an orange oil (1.333 g). LCMS m/z=238.2 [M+H]⁺.

### Step D: Preparation of (R)-3-Fluoro-4-(2-(2-methylpyrrolidin-1-yl)ethyl)phenol

To a solution of (R)-1-(2-fluoro-4-methoxyphenethyl)-2-methylpyrrolidine (1.322 g, 5.57 mmol) in DCM (55.7 mL) was added BBr₃ (1 M in DCM) (13.93 mL, 13.93 mmol). The reaction was stirred at room temperature for 1 h. The reaction mixture was diluted with EtOAc (50 mL), washed with water and brine, dried over Na₂SO₄ and concentrated. The residue was dissolved in DCM and purified by silica gel column chromatography to give the title compound (1.137 g). LCMS m/z=224.3 [M+H]⁺.

### Step E: Preparation of (R)-3-Fluoro-4-(2-(2-methylpyrrolidin-1-yl)ethyl)phenyl trifluoromethanesulfonate

To a solution of (R)-3-fluoro-4-(2-(2-methylpyrrolidin-1-yl)ethyl)phenol (0.046 g, 0.206 mmol) in DCM (55.7 mL) was added pyridine (0.050 mL, 0.618 mmol) followed by trifluoromethanesulfonic anhydride (0.052 mL, 0.309 mmol). The reaction was stirred for 1 h. The reaction mixture was diluted with EtOAc, washed with 1 M HCl (twice), brine, dried over Na₂SO₄ and concentrated to give the title compound without further purification. LCMS m/z=356.2 [M+H]⁺.

### Step F: Preparation of 1-(6-Bromo-3,4-dihydroisoquinolin-2(1H)-yl)ethanone

To a stirred slurry of 6-bromo-1,2,3,4-tetrahydroisoquinoline hydrochloride (2.460 g, 9.90 mmol) in THF (39.6 mL) was added triethylamine (4.14 mL, 29.7 mmol). The reaction mixture was cooled in an ice-bath, and acetyl chloride (0.880 mL, 12.37 mmol) was added slowly. The ice-bath was removed and the mixture was stirred at room temperature for 30 min. The mixture was diluted with ethyl acetate and washed with 1 M HCl and brine. The ethyl acetate layer was dried over sodium sulfate and the solvent was removed under reduced pressure. The residue was purified by silica gel column chromatography to give the title compound (1.983 g). LCMS m/z=256.3 [M+H]⁺. ¹H NMR (400 MHz, CDCl₃) δ ppm 2.17 (d, J=1.52 Hz, 3H), 2.78-2.91 (m, 2H), 3.60-3.86 (m, 2H), 4.53-4.70 (m, 2H), 6.94-7.06 (m, 1H), 7.28-7.36 (m, 2H).

### Step G: Preparation of 1-(6-(4,4,5,5-Tetramethyl-1,3,2-dioxaborolan-2-yl)-3,4-dihydroisoquinolin-2(1H)-yl)ethanone

To a solution of 1-(6-bromo-3,4-dihydroisoquinolin-2(1H)-yl)ethanone (0.322 g, 1.267 mmol) in dimethylsulfoxide (6.34 mL) were added tetrakis(triphenylphosphine)palladium(0) (0.044 g, 0.038 mmol), potassium acetate (0.373 g, 3.80 mmol), and bis(pinacolato)diboron (0.483 g, 1.901 mmol) under an argon atmosphere. The reaction was stirred at 90° C. for 16 h. The mixture was diluted with ethyl acetate and washed with 1 M HCl and brine. The ethyl acetate layer was dried over sodium sulfate and the solvent was removed under reduced pressure. The residue was purified by silica gel column chromatography to give title compound (240 mg). LCMS m/z=302.5 [M+H]⁺; ¹H NMR (400 MHz, Methanol-d₄) δ ppm 1.31-1.38 (s, 12H), 2.17-2.21 (m, 3H), 2.83-2.98 (m, 2H), 3.72-3.81 (m, 2H), 4.67-4.75 (m, 2H), 7.14-7.22 (m, 1H), 7.55-7.60 (m, 2H).

### Step H: Preparation of (R)-1-(6-(3-Fluoro-4-(2-(2-methylpyrrolidin-1-yl)ethyl)phenyl)-3,4-dihydroisoquinolin-2(1H)-yl)ethanone

In a microwave vial were placed 1-(6-(4,4,5,5-tetramethyl-1,3,2-dioxaborolan-2-yl)-3,4-dihydroisoquinolin-2(1H)-yl)ethanone (0.060 g, 0.199 mmol), (R)-3-fluoro-4-(2-(2-methylpyrrolidin-1-yl)ethyl)phenyl trifluoromethanesulfonate (0.071 g, 0.199 mmol), and tetrakis(triphenylphosphine)palladium (0) (6.91 mg, 5.98 μmol). Benzene (3 mL) and EtOH (1 mL) were added followed by Na₂CO₃ (0.199 mL, 0.398 mmol). The reaction was heated under microwave irradiation for 1 h at 120° C. The organic layer was separated and concentrated. The residue was purified by HPLC to give the TFA salt of the title compound (19 mg). LCMS m/z=381.3 [M+H]⁺. ¹H NMR (400 MHz, Methanol-d₄) δ ppm 1.34 (d, J=6.57 Hz, 0.3H), 1.47 (d, J=6.57 Hz, 2.7H), 1.68-1.82 (m, 1H), 2.02-2.19 (m, 2H), 2.17-2.22 (m, 3H), 2.29-2.40 (m, 1H), 2.89-3.02 (m, 2H), 3.07-3.20 (m, 2H), 3.21-3.32 (m, 2H), 3.50-3.68 (m, 2H), 3.74-3.82 (m, 3H), 4.69-4.75 (m, 2H), 7.26 (dd, J=10.67, 8.15 Hz, 1H), 7.38-7.50 (m, 5H).

### Example 1.59

### Preparation of (R)-1-(6-(2-Chloro-4-(2-(2-methylpyrrolidin-1-yl)ethyl)phenyl)-3,4-dihydroisoquinolin-2(1H)-yl)ethanone (Compound 53)

### Step A: Preparation of Methyl 2-(3-Chloro-4-hydroxyphenyl)acetate

To a solution of 2-(3-chloro-4-hydroxyphenyl)acetic acid (10.0 g, 53.6 mmol) in MeOH (250 mL) was added concentrated sulfuric acid. The resulting mixture was heated at reflux for 16 h. MeOH was evaporated to give an oil which was partitioned between water and EtOAc. The aqueous phase was extracted with EtOAc (three times). The combined organic phases were washed with brine, dried over sodium sulfate, and then evaporated to give the title compound as an amber oil (10.5 g). NMR (400 MHz, DMSO-d₆) δ ppm 3.57 (s, 2H), 3.64 (s, 3H), 6.09 (d, J=8.3 Hz, 1H), 7.02 (dd, J=8.3, 2.1 Hz, 1H), 7.23 (d, J=2.1 Hz, 1H), 10.03 (s, 1H).

### Step B: Preparation of Methyl 2-(3-Chloro-4-(4-methoxybenzyloxy)phenyl)acetate

To a solution of methyl 2-(3-chloro-4-hydroxyphenyl)acetate (2.19 g, 10.9 mmol) in acetone (27 mL) was added PMBCl (p-methoxybenzyl chloride) (1.88 g, 12.0 mmol), tetrabutylammonium iodide (TBAI) (4.03 g, 10.9 mmol), and potassium carbonate (2.26 g, 16.4 mmol). The mixture was heated at 55° C. for 60 h. The reaction mixture was diluted with a solution of 10% acetone in hexanes and a small amount of DCM, heated briefly, and then cooled to room temperature and passed through a column of Celite®/silica gel. The column was washed with 10-20% acetone/hexanes (700 mL), and the colorless eluent was concentrated to give the title compound as an off-white solid (3.4 g) with minor impurities. TLC (20% acetone/hexanes) R_{f}=0.27.

### Step C: Preparation of 2-(3-Chloro-4-(4-methoxybenzyloxy)phenyl)ethanol

To a solution of methyl 2-(3-chloro-4-(4-methoxybenzyloxy)phenyl)acetate (0.50 g, 1.6 mmol) cooled to 0° C. in THF (15 mL) was added lithium aluminum hydride (1.6 mL of 1 M THF solution, 1.6 mmol). The cold bath was allowed to expire while the reaction stirred overnight. The reaction was quenched by pouring onto ice, then the slurry was extracted with EtOAc (three times). The combined organic phases were washed with brine, dried over sodium sulfate, and concentrated to give the title compound (0.49 g) with minor impurities.

### Step D: Preparation of 3-Chloro-4-(4-methoxybenzyloxy)phenethyl Methanesulfonate

To a solution of 2-(3-chloro-4-(4-methoxybenzyloxy)phenyl)ethanol (1.096 g, 3.74 mmol) in DCM (7.49 mL) was added triethylamine (0.783 mL, 5.62 mmol). The mixture was cooled in an ice bath and added methanesulfonyl chloride (0.350 mL, 4.49 mmol). The ice-bath was removed and the reaction was stirred at room temperature for 1 h. The mixture was diluted with EtOAc, washed with 1 M HCl (15 mL) and brine, dried over Na₂SO₄ and concentrate to give the title compound (1.522 g) without further purification.

### Step E: Preparation of (R)-1-(3-Chloro-4-(4-methoxybenzyloxy)phenethyl)-2-methylpyrrolidine

3-Chloro-4-(4-methoxybenzyloxy)phenethyl methanesulfonate (1.515 g, 4.09 mmol) was dissolved in acetonitrile (10.21 mL). (R)-2-Methylpyrrolidine benzene sulfonate (1.188 g, 4.90 mmol) and potassium carbonate (1.694 g, 12.26 mmol) were added. The reaction was heated at 60° C. overnight. The mixture was cooled to room temperature and filtered. The filter cake was washed with acetonitrile. The filtrate was concentrated. The residue was dissolved in EtOAc and water, and then treated with 10% HCl to adjust to pH 2. The aqueous phase was washed with EtOAc (twice). The combined EtOAc extracts were concentrated to give the title compound (963 mg). The aqueous layer was treated with 2 M Na₂CO₃ to adjust to pH 9, and extracted with EtOAc (three times). The organic extracts were washed with brine, dried over Na₂SO₄, and concentrated to give the title compound as an orange oil (547 mg). LCMS m/z=360.4 [M+H]⁺.

### Step F: Preparation of (R)-2-Chloro-4-(2-(2-methylpyrrolidin-1-yl)ethyl)phenol

To a solution of (R)-1-(3-chloro-4-(4-methoxybenzyloxy)phenethyl)-2-methylpyrrolidine (0.953 g, 2.65 mmol) in DCM (1.5 mL) was added 2,2,2-trifluoroacetic acid (1.5 mL, 20.19 mmol). The reaction was stirred at room temperature for 2 min. The mixture was quenched with NaHCO₃ (to pH 9) and extracted with DCM (three times). The combined organic extracts were dried over Na₂SO₄ and concentrated. The residue was purified by silica gel column chromatography to give the title compound (353 mg). LCMS m/z=240.1 [M+H]⁺.

### Step G: Preparation of (R)-2-Chloro-4-(2-(2-methylpyrrolidin-1-yl)ethyl)phenyl Trifluoromethanesulfonate

From (R)-2-chloro-4-(2-(2-methylpyrrolidin-1-yl)ethyl)phenol, using a similar method to the one described in Example 1.58, Step E, the title compound was obtained. LCMS m/z=272.2 [M+H]⁺. Step H: Preparation of (R)-1-(6-(2-Chloro-4-(2-(2-methylpyrrolidin-1-yl)ethyl)phenyl)-3,4-dihydroisoquinolin-2(1H)-yl)ethanone

From 1-(6-(4,4,5,5-tetramethyl-1,3,2-dioxaborolan-2-yl)-3,4-dihydroisoquinolin-2(1H)-yl)ethanone and (R)-2-chloro-4-(2-(2-methylpyrrolidin-1-yl)ethyl)phenyl trifluoromethanesulfonate, using a similar method to the one described in Example 1.58, Step H, the TFA salt of the title compound was obtained. LCMS m/z=397.4 [M+H]⁺. ¹H NMR (400 MHz, Methanol-d₄) δ ppm 1.32 (d, J=6.44 Hz, 0.3H), 1.47 (d, J=6.44 Hz, 2.7H), 1.69-1.81 (m, 1H), 2.02-2.18 (m, 2H), 2.20 (d, J=5.43 Hz, 3H), 2.29-2.40 (m, 1H), 2.85-3.01 (m, 2H), 3.01-3.16 (m, 2H), 3.22-3.31 (m, 2H), 3.49-3.57 (m, 1H), 3.59-3.68 (m, 1H), 3.72-3.83 (m, 3H), 4.70-4.76 (m, 2H), 7.18-7.27 (m, 3H), 7.30-7.37 (m, 2H), 7.50 (s, 1H).

### Example 1.60

### Preparation of Intermediate 4-Bromophenethyl Methanesulfonate

4-Bromophenethyl alcohol (38.9 g, 193 mmol) was dissolved in DCM (193 mL). Triethylamine (40.4 mL, 290 mmol) was added and the mixture was cooled in an ice bath. Methanesulfonyl chloride (18 mL, 232 mmol) was added dropwise via an addition funnel. The ice bath was removed and the mixture was stirred for 30 min. The reaction mixture was diluted with DCM (200 mL), washed with 1 M HCl twice (100 mL each), followed by brine, saturated sodium bicarbonate, and brine. The organic phase was dried with sodium sulfate and filtered. The solvent was removed under reduced pressure to give the title compound (54.0 g) in quantitative yield. ¹H NMR (400 MHz, CDCl₃) δ ppm 2.89 (s, 3H), 3.02 (t, J=6.82 Hz, 2H), 4.40 (t, J=6.82 Hz, 2H), 7.03-7.17 (m, 2H), 7.43-7.47 (m, 2H).

### Example 2

### [³H] N-Alpha-Methyl-Histamine Competitive Histamine H3 Receptor Binding Assay

The histamine receptor binding assay was conducted using standard laboratory procedures as described below. A crude membrane fraction was prepared from whole rat brain cortex using a polytron to homogenize the tissue followed by differential centrifugation in a HEPES-based buffer containing protease inhibitors. Membranes where frozen at −80° C. until needed. Frozen membranes were thawed and resuspended in ice-cold assay buffer consisting of 50 mM TRIS containing 5 mM EDTA (pH=7.4). 50 micrograms (μg) of membrane protein was added to each well of a 96-well assay plate along with test compound and [³H]-N-α-methylhistamine (1 nanomolar (nM) final assay concentration). Imetit was used as an assay positive control at varying concentrations. The plate was incubated for 30 min at room temperature. The assay was terminated by rapid filtration through a 96-well glass fiber filtration plate (GF/C) using a cell harvester (Perkin-Elmer). Captured membranes were washed three times with cold assay buffer and plates were dried at 50° C. 35 microliters (μL) of scintillation cocktail was added to each well and membrane-bound radioactivity was recorded using a TopCount 96-well plate scintillation counter (Perkin-Elmer).

The following table shows the observed activities for certain compounds of the present invention.

| Compound No. | Kᵢ Binding Assay (nM) |
|---|---|
| 3 | 0.45 |
| 5 | 0.11 |
| 9 | 1.1 |
| 12 | 0.09 |
| 16 | 1.52 |

Certain other compounds of the invention had activity values ranging from 0.09 nM to 1.52 nM in this assay.

### Example 3

### Human Histamine H3 Receptor Binding Assay—MDS Pharma Services (Taiwan)

Compounds of the invention were tested for their ability to bind to the human histamine H3 receptor using the MDS Pharma Services (Taiwan) assay, Catalogue No. 239810. Certain compounds of the present invention and their corresponding activity values are shown in following table.

| Compound No. | Binding Assay (Ki, nM) |
|---|---|
| 5 | 0.57 |
| 18 | 2.15 |

Certain other compounds of the invention had activity values ranging from about 0.53 nM to about 2.87 nM in this assay.

### Example 4

### Blockade of RAMH-Induced Drinking Assay

When administered to rodents, histamine H3 receptor agonists such as (R)-α-methylhistamine (RAMH) induce a drinking response that is sensitive to reversal with a histamine H3 receptor antagonist. Blockade of RAMH-induced drinking can therefore be utilized as an in vivo assay for functional histamine H3 receptor antagonist activity. In this assay, male Sprague Dawley rats (250-350 g) were housed three per cage and maintained under a reverse 12 h light cycle (lights off at 1130 h). At 1030 h on the day of test, rats were individually housed in new cages and food was removed. 120 min later, rats were administered test article (vehicle or histamine H3 receptor antagonist, 0.3 mg/kg PO). 30 min later, water was removed, and RAMH (vehicle or RAMH 3 mg/kg salt SC) was administered. 10 min after administration of RAMH, weighed water bottles were placed in the cages, and drinking was allowed for 20 min. Water consumption was determined for each animal by weighing each bottle to the nearest 0.1 g. Data is expressed as percentage reduction in water intake according to the following formula:

[((VEH/RAMH)−(ANTAGONIST/RAMH))/((VEH/RAMH)−(VEH/VEH))]*100

| Compound No. | % inhibition of RAMH-induced drinking |
|---|---|
| 1 | 81.6 |
| 14 | 40.7 |
| 18 | 66.1 |

### Example 5

### Powder X-Ray Diffraction

Powder X-ray Diffraction (PXRD) data were collected on an X'Pert PRO MPD powder diffractometer (PANalytical, Inc.) with a Cu source set at 45 kV and 40 mA, a Ni-filter to remove Cu Kβ radiation, and an X'Celerator detector. The instrument was calibrated by the vendor using a silicon powder standard NIST #640c. The calibration was found to be correct when it was tested with NIST #675 low-angle diffraction standard. Samples were prepared for PXRD scanning by placing several milligrams of as-is compound onto a sample holder and smoothing as flat as possible by pressing weigh paper down on the sample with a flat object. The samples were analyzed using a spinning-sample stage. Scans covered the range of 5 to 40°2θ. A continuous scan mode was used with a step size of 0.0170°2θ. Diffraction data were viewed and analyzed with the X'Pert Data Viewer Software, version 1.0a and X'Pert HighScore Software, version 1.0b. The PXRD pattern for the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride is shown in FIG. 14.

### Example 6

### Differential Scanning Calorimetry

Differential Scanning Calorimetry (DSC) was performed on a TA instruments, Inc. DSC Q2000 at 10° C./min. The instrument was calibrated at this scan rate by the vendor for temperature and energy using the melting point and enthalpy of fusion of an indium standard. Samples were prepared by taring a sample-pan lid along with a sample-pan bottom on a Mettler Toldeo MX5 balance. Sample was placed in the bottom of the tared sample pan. The sample-pan lid fitted snuggly in the sample-pan bottom. The sample and pan were reweighed to get the sample weight. Thermal events (for example, onset temperature, enthalpy of fusion) are calculated using the Universal Analysis 2000 software, version 4.1D, Build 4.1.0.16. The DSC thermogram for the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride is shown in FIG. 15 overlaid with the TGA trace.

### Example 7

### Thermal Gravimetric Analysis

Thermal Gravimetric Analysis (TGA) was performed on the TA Instruments, Inc. TGA Q5000. The instrument is calibrated by the vendor at 10° C./min. for temperature using the curie point of a ferromagnetic standard. The balance is calibrated with a standard weight. Sample scans are performed at 10° C./min. Sample is placed into an open sample pan, previously tared on the TGA balance. Thermal events such as weight-loss are calculated using the Universal Analysis 2000 software, version 4.1D, Build 4.1.0.16. The TGA thermogram for the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride is shown in FIG. 14 overlaid with the DSC trace.

### Example 8

### Dynamic Vapor Sorption (DVS)

Hygroscopicity was measured using a dynamic moisture-sorption analyzer, VTI Corporation, SGA-100. The sample was placed as-is in a tared sample holder on the VTI balance. A drying step was run at 40° C. and ˜1% RH for 120 minutes. The isotherm conditions are 25° C. with steps of 20% RH from 10% RH up to 90% RH and back to 10% RH. Weight is checked every 2 minutes. Percent weight change of <0.01% in 20 minutes or 2 hours, whichever occurs first, is required before continuing to the next step. The DVS profile for the crystalline form (Form 1) of (R)-2-hydroxy-1-(6-(4-(2-(2-methylpyrrolidin-1-yl)ethyl)phenyl)-3,4-dihydroisoquinolin-2(1H)-yl)ethanone hydrochloride is shown in FIG. 16.

Those skilled in the art will recognize that various modifications, additions, substitutions and variations to the illustrative examples set forth herein can be made without departing from the spirit of the invention and are, therefore, considered within the scope of the invention. All documents referenced above, including, but not limited to, printed publications and provisional and regular patent applications, are incorporated herein by reference in their entirety.

1. A compound selected from compounds of Formula (Ia) and pharmaceutically acceptable salts, solvates and hydrates thereof: 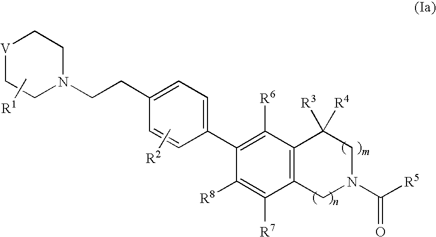
wherein:
R¹ is H or C₁-C₄ alkyl;
R² is H or halogen;
R³ is H, C₁-C₄ alkyl or C₃-C₆ cycloalkyl, and R⁴ is H; or R³ and R⁴ together with the atom to which they are both bonded form a C₃-C₆ cycloalkyl;
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;
R⁶, R⁷ and R⁸ are each independently selected from: H, C₁-C₆ alkoxy, C₁-C₆ alkyl, amino, halogen, heterocyclyl and hydroxyl;
m is 0 or 1;
n is 1 or 2; and
V is CH₂, O or absent.

2. A compound according to claim 1, wherein R¹ is H.

3. A compound according to claim 1, wherein R¹ is methyl.

4. A compound according to claim 1, wherein R² is H.

5. A compound according to claim 1, wherein R² is fluoro or chloro.

6. A compound according to claim 1, wherein R³ is C₁-C₄ alkyl and R⁴ is H.

7. A compound according to claim 1, wherein R³ is methyl and R⁴ is H.

8. A compound according to claim 1, wherein R³ and R⁴ are both H.

9. A compound according to claim 1, wherein R⁵ is selected from: methyl, ethyl, n-propyl, cyclopropyl, phenyl, pyridyl, pyrimidinyl and tetrahydropyranyl; each of which is optionally substituted with one or more substituents selected from: C₁-C₆ alkoxy, halogen, heterocyclyl and hydroxyl.

10. A compound according to claim 1, wherein R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl, 2-hydroxypyridin-4-yl, 6-hydroxypyridin-2-yl and 6-methoxypyridin-3-yl.

11. A compound according to claim 1, wherein R⁶, R⁷ and R⁸ are each independently selected from: H, methoxy, methyl, fluoro, chloro, bromo and hydroxyl.

12. A compound according to claim 1, wherein R⁶, R⁷ and R⁸ are all H.

13. A compound according to claim 1, wherein m is 0.

14. A compound according to claim 1, wherein m is 1.

15. A compound according to claim 1, wherein n is 1.

16. A compound according to claim 1, wherein n is 2.

17. A compound according to claim 1, wherein V is O.

18. A compound according to claim 1, wherein V is CH₂.

19. A compound according to claim 1, wherein V is absent.

20. A compound according to claim 1, selected from compounds of Formula (Ic) and pharmaceutically acceptable salts, solvates and hydrates thereof: 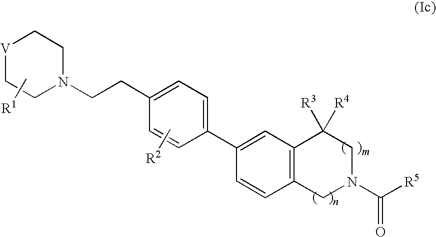
wherein:
R¹ is H or C₁-C₄ alkyl;
R² is H or halogen;
R³ is H, C₁-C₄ alkyl or C₃-C₆ cycloalkyl, and R⁴ is H; or R³ and R⁴ together with the atom to which they are both bonded form a C₃-C₆ cycloalkyl;
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl;
m is 0 or 1;
n is 1 or 2; and
V is CH₂, O or absent.

21. A compound according to claim 1, selected from compounds of Formula (Ic) and pharmaceutically acceptable salts, solvates and hydrates thereof: 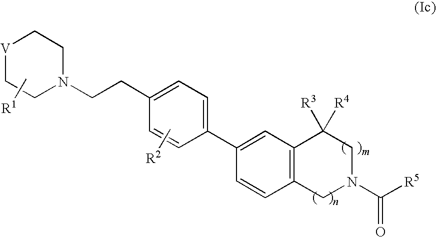
wherein:
R¹ is H or methyl;
R² is H, fluoro or chloro;
R³ is H or methyl;
R⁴ is H;
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl;
m is 0 or 1;
n is 1 or 2; and
V is CH₂ or absent.

22. A compound according to claim 1, selected from compounds of Formula (Ii) and pharmaceutically acceptable salts, solvates and hydrates thereof: 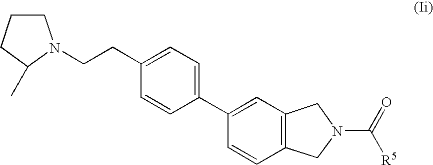
wherein:
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

23. A compound according to claim 1, selected from compounds of Formula (Ii) and pharmaceutically acceptable salts, solvates and hydrates thereof: 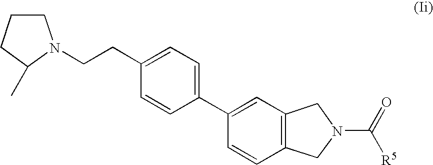
wherein:
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

24. A compound according to claim 1, selected from compounds of Formula (Ik) and pharmaceutically acceptable salts, solvates and hydrates thereof: 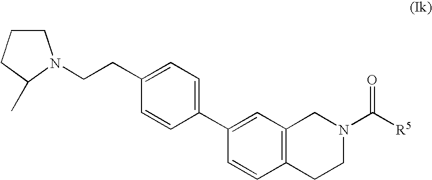
wherein:
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

25. A compound according to claim 1, selected from compounds of Formula (Ik) and pharmaceutically acceptable salts, solvates and hydrates thereof: 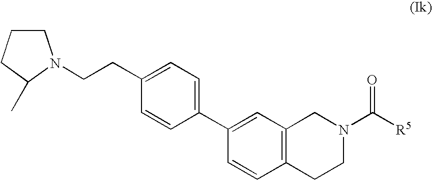
wherein:
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

26. A compound according to claim 1, selected from compounds of Formula (Im) and pharmaceutically acceptable salts, solvates and hydrates thereof: 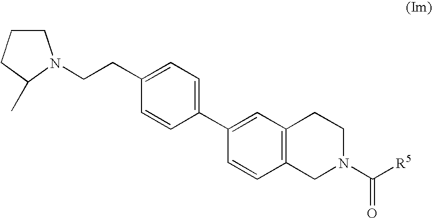
wherein:
R⁵ is selected from: C₁-C₆ alkyl, aryl, C₃-C₆ cycloalkyl, heteroaryl and heterocyclyl; each of which is optionally substituted with one or more substituents selected from: C₁-C₆ alkoxy, halogen, heterocyclyl and hydroxyl.

27. A compound according to claim 1, selected from compounds of Formula (Im) and pharmaceutically acceptable salts, solvates and hydrates thereof: 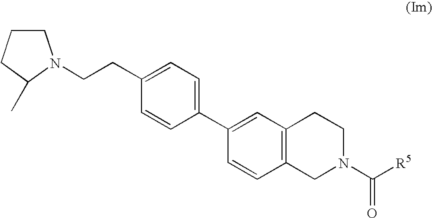
wherein:
R⁵ is selected from: methyl, cyclopropyl, tetrahydropyran-4-yl, methoxymethyl, 2-methoxyethyl, 3-methoxypropyl, hydroxymethyl, 2-hydroxyethyl, tetrahydropyran-4-ylmethyl, 2,2-difluorocyclopropyl, 4-methoxyphenyl, pyridin-2-yl, pyridin-3-yl, pyridin-4-yl, pyrimidin-5-yl, 6-hydroxypyridin-3-yl and 2-hydroxypyridin-4-yl.

28. A compound according to claim 1, selected from the following compounds and pharmaceutically acceptable salts, solvates and hydrates thereof:
(1) (R)-3-methoxy-1-(7-(4-(2-(2-methylpyrrolidin-1-yl)ethyl)phenyl)-3,4-dihydroisoquinolin-2(1H)-yl)propan-1-one;
(2) (R)-cyclopropyl(7-(4-(2-(2-methylpyrrolidin-1-yl)ethyl)phenyl)-3,4-dihydroisoquinolin-2(1H)-yl)methanone;
(3) (R)-cyclopropyl(6-(4-(2-(2-methylpyrrolidin-1-yl)ethyl)phenyl)-3,4-dihydroisoquinolin-2(1H)-yl)methanone;
(4) (R)-3-methoxy-1-(6-(4-(2-(2-methylpyrrolidin-1-yl)ethyl)phenyl)-3,4-dihydroisoquinolin-2(1H)-yl)propan-1-one;
(5) (R)-cyclopropyl(5-(4-(2-(2-methylpyrrolidin-1-yl)ethyl)phenyl)isoindolin-2-yl)methanone;
(6) (R)-3-methoxy-1-(5-(4-(2-(2-methylpyrrolidin-1-yl)ethyl)phenyl)isoindolin-2-yl)propan-1-one;
(7) (R)-(6-(4-(2-(2-methylpyrrolidin-1-yl)ethyl)phenyl)-3,4-dihydroisoquinolin-2(1H)-yl)(tetrahydro-2H-pyran-4-yl)methanone;
(8) (2,2-difluorocyclopropyl)(6-(4-(2-((R)-2-methylpyrrolidin-1-yl)ethyl)phenyl)-3,4-dihydroisoquinolin-2(1H)-yl)methanone;
(9) (R)-(4-methoxyphenyl)(6-(4-(2-(2-methylpyrrolidin-1-yl)ethyl)phenyl)-3,4-dihydroisoquinolin-2(1H)-yl)methanone;
(10) (R)-2-hydroxy-1-(6-(4-(2-(2-methylpyrrolidin-1-yl)ethyl)phenyl)-3,4-dihydroisoquinolin-2(1H)-yl)ethanone;
(11) (R)-1-(6-(4-(2-(2-methylpyrrolidin-1-yl)ethyl)phenyl)-3,4-dihydroisoquinolin-2(1H)-yl)-2-(tetrahydro-2H-pyran-4-yl)ethanone;
(12) (R)-(5-(4-(2-(2-methylpyrrolidin-1-yl)ethyl)phenyl)isoindolin-2-yl)(tetrahydro-2H-pyran-4-yl)methanone;
(13) (R)-(6-(4-(2-(2-methylpyrrolidin-1-yl)ethyl)phenyl)-3,4-dihydroisoquinolin-2(1H)-yl)(pyridin-3-yl)methanone;
(14) (R)-(6-(4-(2-(2-methylpyrrolidin-1-yl)ethyl)phenyl)-3,4-dihydroisoquinolin-2(1H)-yl)(pyridin-4-yl)methanone;
(15) (R)-(6-(4-(2-(2-methylpyrrolidin-1-yl)ethyl)phenyl)-3,4-dihydroisoquinolin-2(1H)-yl)(pyrimidin-5-yl)methanone;
(16) (R)-3-hydroxy-1-(6-(4-(2-(2-methylpyrrolidin-1-yl)ethyl)phenyl)-3,4-dihydroisoquinolin-2(1H)-yl)propan-1-one;
(17) (R)-4-methoxy-1-(5-(4-(2-(2-methylpyrrolidin-1-yl)ethyl)phenyl)isoindolin-2-yl)butan-1-one;
(18) (R)-(6-(4-(2-(2-methylpyrrolidin-1-yl)ethyl)phenyl)-3,4-dihydroisoquinolin-2(1H)-yl)(pyridin-2-yl)methanone;
(19) (R)-2-methoxy-1-(6-(4-(2-(2-methylpyrrolidin-1-yl)ethyl)phenyl)-3,4-dihydroisoquinolin-2(1H)-yl)ethanone;
(20) (R)-4-methoxy-1-(6-(4-(2-(2-methylpyrrolidin-1-yl)ethyl)phenyl)-3,4-dihydroisoquinolin-2(1H)-yl)butan-1-one;
(21) (R)-(6-hydroxypyridin-3-yl)(6-(4-(2-(2-methylpyrrolidin-1-yl)ethyl)phenyl)-3,4-dihydroisoquinolin-2(1H)-yl)methanone;
(22) (R)-(2-hydroxypyridin-4-yl)(6-(4-(2-(2-methylpyrrolidin-1-yl)ethyl)phenyl)-3,4-dihydroisoquinolin-2(1H)-yl)methanone; and
(23) (R)-1-(6-(4-(2-(2-methylpyrrolidin-1-yl)ethyl)phenyl)-3,4-dihydroisoquinolin-2(1H)-yl)ethanone.

29. A compound according to claim 1, selected from the following compounds and pharmaceutically acceptable salts, solvates and hydrates thereof:
(24) 1-((R)-1-methyl-8-(4-(2-((R)-2-methylpyrrolidin-1-yl)ethyl)phenyl)-4,5-dihydro-1H-benzo[d]azepin-3(2H)-yl)ethanone;
(25) (R)-(5-(4-(2-(2-methylpyrrolidin-1-yl)ethyl)phenyl)isoindolin-2-yl)(pyridin-4-yl)methanone;
(26) 3-methoxy-1-((R)-1-methyl-8-(4-(2-((R)-2-methylpyrrolidin-1-yl)ethyl)phenyl)-4,5-dihydro-1H-benzo[d]azepin-3(2H)-yl)propan-1-one;
(27) cyclopropyl((R)-1-methyl-8-(4-(2-((R)-2-methylpyrrolidin-1-yl)ethyl)phenyl)-4,5-dihydro-1H-benzo[d]azepin-3(2H)-yl)methanone;
(28) (R)-(5-(4-(2-(2-methylpyrrolidin-1-yl)ethyl)phenyl)isoindolin-2-yl)(pyridin-3-yl)methanone;
(29) (R)-(5-(4-(2-(2-methylpyrrolidin-1-yl)ethyl)phenyl)isoindolin-2-yl)(pyrimidin-5-yl)methanone;
(30) (R)-(5-(4-(2-(2-methylpyrrolidin-1-yl)ethyl)phenyl)isoindolin-2-yl)(pyridin-2-yl)methanone;
(31) (R)-(6-hydroxypyridin-2-yl)(6-(4-(2-(2-methylpyrrolidin-1-yl)ethyl)phenyl)-3,4-dihydroisoquinolin-2(1H)-yl)methanone;
(32) (R)-(6-methoxypyridin-3-yl)(6-(4-(2-(2-methylpyrrolidin-1-yl)ethyl)phenyl)-3,4-dihydroisoquinolin-2(1H)-yl)methanone;
(33) (R)-2-methoxy-1-(5-(4-(2-(2-methylpyrrolidin-1-yl)ethyl)phenyl)isoindolin-2-yl)ethanone;
(34) (R)-2-hydroxy-1-(5-(4-(2-(2-methylpyrrolidin-1-yl)ethyl)phenyl)isoindolin-2-yl)ethanone;
(35) 1-((R)-9-fluoro-1-methyl-8-(4-(2-((R)-2-methylpyrrolidin-1-yl)ethyl)phenyl)-4,5-dihydro-1H-benzo[d]azepin-3(2H)-yl)ethanone;
(37) (R)-1-(5-chloro-6-(4-(2-(2-methylpyrrolidin-1-yl)ethyl)phenyl)-3,4-dihydroisoquinolin-2(1H)-yl)ethanone;
(38) (R)-1-(5-fluoro-6-(4-(2-(2-methylpyrrolidin-1-yl)ethyl)phenyl)-3,4-dihydroisoquinolin-2(1H)-yl)ethanone;
(39) 1-(7-hydroxy-1-methyl-8-(4-(2-((R)-2-methylpyrrolidin-1-yl)ethyl)phenyl)-4,5-dihydro-1H-benzo[d]azepin-3(2H)-yl)ethanone;
(40) (R)-1-(7-methyl-6-(4-(2-(2-methylpyrrolidin-1-yl)ethyl)phenyl)-3,4-dihydroisoquinolin-2(1H)-yl)ethanone;
(41) 1-(7-methoxy-1-methyl-8-(4-(2-((R)-2-methylpyrrolidin-1-yl)ethyl)phenyl)-4,5-dihydro-1H-benzo[d]azepin-3(2H)-yl)ethanone;
(42) (R)-1-(7-(4-(2-(2-methylpyrrolidin-1-yl)ethyl)phenyl)-4,5-dihydro-1H-benzo[d]azepin-3(2H)-yl)ethanone;
(43) 2-hydroxy-1-(6-(4-(2-(piperidin-1-yl)ethyl)phenyl)-3,4-dihydroisoquinolin-2(1H)-yl)ethanone;
(44) (R)-1-(7-methoxy-6-(4-(2-(2-methylpyrrolidin-1-yl)ethyl)phenyl)-3,4-dihydroisoquinolin-2(1H)-yl)ethanone;
(45) 2-hydroxy-1-(6-(4-(2-morpholinoethyl)phenyl)-3,4-dihydroisoquinolin-2(1H)-yl)ethanone;
(46) (R)-1-(7-hydroxy-6-(4-(2-(2-methylpyrrolidin-1-yl)ethyl)phenyl)-3,4-dihydroisoquinolin-2(1H)-yl)ethanone;
(48) (R)-2-hydroxy-1-(7-(4-(2-(2-methylpyrrolidin-1-yl)ethyl)phenyl)-4,5-dihydro-1H-benzo[d]azepin-3(2H)-yl)ethanone;
(49) (R)-1-(7-fluoro-6-(4-(2-(2-methylpyrrolidin-1-yl)ethyl)phenyl)-3,4-dihydroisoquinolin-2(1H)-yl)ethanone;
(50) 2-hydroxy-1-(6-(4-(2-(2-methylpyrrolidin-1-yl)ethyl)phenyl)-3,4-dihydroisoquinolin-2(1H)-yl)ethanone;
(51) 1-(4-methyl-6-(4-(2-((R)-2-methylpyrrolidin-1-yl)ethyl)phenyl)-3,4-dihydroisoquinolin-2(1H)-yl)ethanone;
(52) (R)-1-(6-(3-fluoro-4-(2-(2-methylpyrrolidin-1-yl)ethyl)phenyl)-3,4-dihydroisoquinolin-2(1H)-yl)ethanone; and
(53) (R)-1-(6-(2-chloro-4-(2-(2-methylpyrrolidin-1-yl)ethyl)phenyl)-3,4-dihydroisoquinolin-2(1H)-yl)ethanone.

30. A compound according to claim 1, selected from the following compound and pharmaceutically acceptable salts, solvates and hydrates thereof:
(3) (R)-cyclopropyl(6-(4-(2-(2-methylpyrrolidin-1-yl)ethyl)phenyl)-3,4-dihydroisoquinolin-2(1H)-yl)methanone.

31. A compound according to claim 1, selected from the following compound and pharmaceutically acceptable salts, solvates and hydrates thereof:
(4) (R)-3-methoxy-1-(6-(4-(2-(2-methylpyrrolidin-1-yl)ethyl)phenyl)-3,4-dihydroisoquinolin-2(1H)-yl)propan-1-one.

32. A compound according to claim 1, selected from the following compound and pharmaceutically acceptable salts, solvates and hydrates thereof:
(7) (R)-(6-(4-(2-(2-methylpyrrolidin-1-yl)ethyl)phenyl)-3,4-dihydroisoquinolin-2(1H)-yl)(tetrahydro-2H-pyran-4-yl)methanone.

33. A compound according to claim 1, selected from the following compound and pharmaceutically acceptable salts, solvates and hydrates thereof:
(10) (R)-2-hydroxy-1-(6-(4-(2-(2-methylpyrrolidin-1-yl)ethyl)phenyl)-3,4-dihydroisoquinolin-2(1H)-yl)ethanone.

34. A compound according to claim 1, selected from the following compound and pharmaceutically acceptable salts, solvates and hydrates thereof:
(11) (R)-1-(6-(4-(2-(2-methylpyrrolidin-1-yl)ethyl)phenyl)-3,4-dihydroisoquinolin-2(1H)-yl)-2-(tetrahydro-2H-pyran-4-yl)ethanone.

35. A compound according to claim 1, selected from the following compound and pharmaceutically acceptable salts, solvates and hydrates thereof:
(16) (R)-3-hydroxy-1-(6-(4-(2-(2-methylpyrrolidin-1-yl)ethyl)phenyl)-3,4-dihydroisoquinolin-2(1H)-yl)propan-1-one.

36. A compound according to claim 1, selected from the following compound and pharmaceutically acceptable salts, solvates and hydrates thereof:
(19) (R)-2-methoxy-1-(6-(4-(2-(2-methylpyrrolidin-1-yl)ethyl)phenyl)-3,4-dihydroisoquinolin-2(1H)-yl)ethanone.

37. A compound according to claim 1, selected from the following compound and pharmaceutically acceptable salts, solvates and hydrates thereof:
(23) (R)-1-(6-(4-(2-(2-methylpyrrolidin-1-yl)ethyl)phenyl)-3,4-dihydroisoquinolin-2(1H)-yl)ethanone.

38. A compound according to claim 1, selected from the following compound and pharmaceutically acceptable salts, solvates and hydrates thereof:
(34) (R)-2-hydroxy-1-(5-(4-(2-(2-methylpyrrolidin-1-yl)ethyl)phenyl)isoindolin-2-yl)ethanone.

39. A compound according to claim 1, selected from the following compound and pharmaceutically acceptable salts, solvates and hydrates thereof:
(50) 2-hydroxy-1-(6-(4-(2-(2-methylpyrrolidin-1-yl)ethyl)phenyl)-3,4-dihydroisoquinolin-2(1H)-yl)ethanone.

40. A compound according to claim 1, wherein the compound is:
(R)-2-hydroxy-1-(6-(4-(2-(2-methylpyrrolidin-1-yl)ethyl)phenyl)-3,4-dihydroisoquinolin-2(1H)-yl)ethanone hydrochloride.

41. A compound according to claim 1 in crystalline form, wherein the compound is:
(R)-2-hydroxy-1-(6-(4-(2-(2-methylpyrrolidin-1-yl)ethyl)phenyl)-3,4-dihydroisoquinolin-2(1H)-yl)ethanone hydrochloride.

42. A crystalline form of (R)-2-hydroxy-1-(6-(4-(2-(2-methylpyrrolidin-1-yl)ethyl)phenyl)-3,4-dihydroisoquinolin-2(1H)-yl)ethanone hydrochloride (Form 1) having an X-ray powder diffraction pattern substantially as shown in FIG. 14.

43. The crystalline form according to claim 42 having a dynamic vapor sorption profile substantially as shown in FIG. 16.

44. The crystalline form according to claim 42 having a differential scanning calorimetry thermogram substantially as shown in FIG. 15.

45. A pharmaceutical composition comprising a compound according to claim 1 and a pharmaceutically acceptable carrier.

46. A method of inducing wakefulness in an individual comprising administering to said individual in need thereof a therapeutically effective amount of a compound according to claim 1.

47. A method for treating a histamine H3 receptor-associated disorder in an individual comprising administering to said individual in need thereof a therapeutically effective amount of a compound according to claim 1.

48. A method for treating a histamine H3 receptor-associated disorder selected from: a cognitive disorder, epilepsy, brain trauma, depression, obesity, a disorder of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, an allergy, an allergic response in the upper airway, allergic rhinitis, nasal congestion, dementia, Alzheimer's disease and pain in an individual comprising administering to said individual in need thereof a therapeutically effective amount of a compound according to claim 1.

49. A method for treating a cognitive disorder in an individual comprising administering to said individual in need thereof a therapeutically effective amount of a compound according to claim 1.

50. A method for treating narcolepsy in an individual comprising administering to said individual in need thereof a therapeutically effective amount of a compound according to claim 1.

51. A method for treating cataplexy in an individual comprising administering to said individual in need thereof a therapeutically effective amount of a compound according to claim 1.

52. A method for treating a histamine H3 receptor-associated disorder selected from: shift-work sleep disorder, jet lag, excessive daytime sleepiness, attention deficit hyperactivity disorder, schizophrenia and pain in an individual comprising administering to said individual in need thereof a therapeutically effective amount of a compound according to claim 1.

53-67. (canceled)

68. A process for preparing a composition comprising admixing a compound according to claim 1.

69. A method of inducing wakefulness in an individual comprising administering to said individual in need thereof a therapeutically effective amount of a crystalline form according to claim 42.

70. A method for treating a histamine H3 receptor-associated disorder in an individual comprising administering to said individual in need thereof a therapeutically effective amount of a crystalline form according to claim 42.

71. A method for treating a histamine H3 receptor-associated disorder selected from: a cognitive disorder, epilepsy, brain trauma, depression, obesity, a disorder of sleep and wakefulness, narcolepsy, shift-work sleep disorder, cataplexy, hypersomnia, somnolence syndrome, jet lag, sleep apnea, excessive daytime sleepiness, attention deficit hyperactivity disorder (ADHD), schizophrenia, an allergy, an allergic response in the upper airway, allergic rhinitis, nasal congestion, dementia, Alzheimer's disease and pain in an individual comprising administering to said individual in need thereof a therapeutically effective amount of a crystalline form according to claim 42.

72. A method for treating a cognitive disorder in an individual comprising administering to said individual in need thereof a therapeutically effective amount of a crystalline form according to claim 42.

73. A method for treating narcolepsy in an individual comprising administering to said individual in need thereof a therapeutically effective amount of a crystalline form according to claim 42.

74. A method for treating cataplexy in an individual comprising administering to said individual in need thereof a therapeutically effective amount of a crystalline form according to claim 42.

75. A method for treating a histamine H3 receptor-associated disorder selected from: shift-work sleep disorder, jet lag, excessive daytime sleepiness, attention deficit hyperactivity disorder, schizophrenia and pain in an individual comprising administering to said individual in need thereof a therapeutically effective amount of a crystalline form according to claim 42.
